(19) United States
(12) Patent Application Publication
(10) Pub. No.: US 2024/0343665 A1
(43) Pub. Date: Oct. 17, 2024

(54) CYCLOOCTENE-BENZOPHENONE MONOMER, AS WELL AS CATIONIC POLYMER, CROSS-LINKED POLYELECTROLYTE, COMPOSITE MATERIAL, MEMBRANE, ELECTRODE AND ELECTROCHEMICAL DEVICE, E.G. ELECTROLYZER, PREPARED THEREFROM

(71) Applicant: ECOLECTRO, INC., Ithaca, NY (US)

(72) Inventors: Kristina Hugar, Ithaca, NY (US); Ryan Selhorst, Ithaca, NY (US); Sarah Louise Poole, Marion, VA (US); Christopher Simoneau, Ithaca, NY (US)

(21) Appl. No.: 18/682,488

(22) PCT Filed: Aug. 10, 2022
(86) PCT No.: PCT/US22/39908

§ 371 Date: Feb. 9, 2024

Related U.S. Application Data
(60) Provisional application No. 63/231,491, filed on Aug. 10, 2021.

(51) Int. Cl. C07C 49/786; C08F 230/02; H01M 8/10; H01M 8/1034; H01M 8/1041; H01M 8/1072; H01M 50/417

(52) U.S. Cl. CPC C07C 49/786; C08F 230/02; H01M 8/1034; H01M 8/1041; H01M 8/1072; H01M 50/417; H01M 2008/1095

(57) ABSTRACT

The present disclosure relates to cross-linked polyelectrolytes comprising polyelectrolyte, and composite materials comprising said cross-linked polyelectrolytes. The present disclosure further relates to membrane electrode assemblies comprising the cross-linked polyelectrolytes and composites of the disclosure, and electrochemical devices comprising the disclosed membrane electrode assemblies.

### RELATED APPLICATIONS

This application claims the benefit of U.S. Provisional Application No. 63/231,491 filed on Aug. 10, 2021. The entire teachings of the above application are incorporated herein by reference.

### GOVERNMENT SUPPORT

This invention was made with Government support under Grant No. 1951215 awarded by the National Science Foundation (NSF) and Grant No. DE-AR0001058 awarded by Advanced Research Projects Agency-Energy (ARPA-E). The Government has certain rights in the invention.

### BACKGROUND OF THE INVENTION

Polymer electrolytes presently used in fuel cells, electrolyzers, redox flow batteries and water purification have low durability, mechanical strength and conductivity. Current materials are not optimized for performance, durability and costs, which reduces the commercial viability of new technologies. Therefore, high-performance polymer electrolytes that are characterized by high ionic conductivity and durability under harsh chemical conditions and in high temperatures are needed.

### SUMMARY OF THE INVENTION

In a first embodiment, the present invention is a compound represented by structural formula (I):

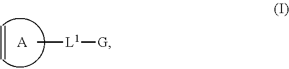

wherein:
the moiety represented by

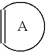

is a C₇₋₈cycloalkenyl or a 7 to 12-membered heterocycloalkenyl,
L¹ is selected from a (O—C₁₋₁₂ alkylene)ₖ, (C₁₋₁₂ alkylene-O)ₖ, C₁₋₁₂ alkylene, C₆₋₁₂ arylene, C₆₋₁₂ arylene-C₁₋₁₂ alkylene, C₁₋₁₂ alkylene-C₆₋₁₂ arylene, C₁₋₁₂ alkylene-O—C₁₋₁₂ alkylene, C₁₋₁₂ alkylene-NH—C₁₋₁₂ alkylene, C₁₋₁₂ alkylene-N(C₁₋₁₂ alkyl)-C₁₋₁₂ alkylene, (NH—C₁₋₁₂ alkylene)ₖ, (C₁₋₁₂ alkylene-NH)ₖ;
the moiety represented by G is selected from a moiety represented by any one of the following structural formulas

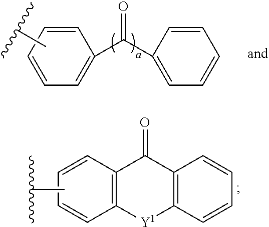

[symbol] s a point of attachment of the moiety represented by G to L¹;
k is an integer between 1 and 6;
a is 1 or 2;
Y¹ is —C(═O)—, O, S, NH, N(C₁₋₁₂ alkyl), or a bond; and
and further wherein:
each C₁₋₁₂ alkylene, C₆₋₁₂ arylene, C₁₋₁₂ alkyl, C₇₋₈cycloalkenyl, and 7 to 12-membered heterocycloalkenyl is independently optionally substituted with 1 to 6 substituents selected from the group consisting of F, Cl, Br, OH, NH₂, C₁₋₁₂ alkyl, C₆₋₁₂ aryl a C₁₋₁₂ haloalkyl, C₁₋₁₂ alkoxy, C₆₋₁₂ aryl, C₆₋₁₂ aryloxy, NH(C₁₋₁₂ alkyl), N(C₁₋₁₂ alkyl)₂, C(O)O(C₁₋₁₂ alkyl), and C(O)NH(C₁₋₁₂ alkyl).

In a second embodiment, the present invention is a polymer, comprising: a plurality of first repeat units represented by structural formula (II):

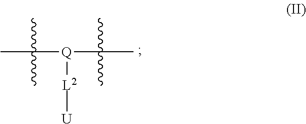

and a plurality of second repeat units represented by structural formula (III):

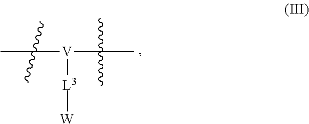

wherein:
Q is a moiety represented by one of the following structural formulas:

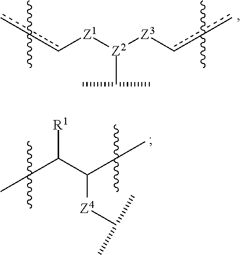

U is a moiety represented by one of the following structural formulas:

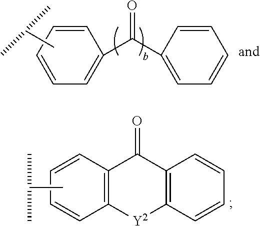

V is a moiety represented by one of the following structural formulas:

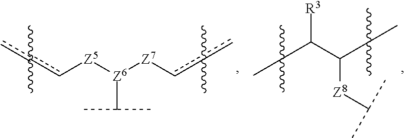

W is a C₁₋₁₂ alkyl or a moiety represented by one of the structural formulas selected from:

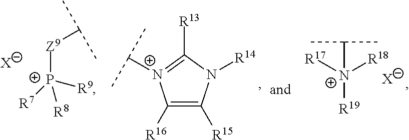

[symbol] is a point of attachment to adjacent repeat units of the polymer;
[symbol] is a point of attachment to L²;
[symbol] is a point of attachment to L³
   and further wherein:
[symbol] is a double bond or a single bond;
Z¹, Z³, Z⁵, and Z⁷ each independently is a C₁₋₃ alkylene or a bond;
Z² is selected from —CHR⁵—, a C₅₋₁₂ cycloalkylene, and a 5 to 16-membered heterocyclylene;
Z⁴ is selected from a bond, a C₆₋₁₂ arylene, and CR², in which case R¹ and R² together with the carbon atoms to which they are attached form a C₅₋₁₂ cycloalkyl or a 5 to 12-membered heterocyclyl;
Z⁶ is selected from —CHR⁶—, a C₅₋₁₂ cycloalkylene, and a 5 to 16-membered heterocyclylene;
Z⁸ is selected from a bond, a C₆₋₁₂ arylene, and CR⁴, in which case R³ and R⁴ together with the carbon atoms to which they are attached form a C₅₋₁₂ cycloalkyl or a 5 to 12 membered heterocyclyl;
Z⁹ is NR¹⁰ or a bond;
R¹, if present, and R³, if present, each independently is selected from H, a C₁₋₁₂ alkyl, and C₆₋₁₂ aryl;
R⁵ and R⁶ each independently is H or a C₁₋₁₂ alkyl;
R⁷, R⁸, and R⁹ each independently is selected from NR¹¹R¹², a C₆₋₁₂ aryl, and 5 to 12-membered heterocyclyl;
R¹⁰ is a C₁₋₁₂ alkyl;
R¹¹ and R¹² each independently is C₁₋₁₂ alkyl or a C₃₋₁₂ cycloalkyl, or R¹¹ and R¹² together with the nitrogen atom to which they are attached form a 5 to 12-membered heterocyclyl;
R¹³ is selected from a C₁₋₁₂ alkyl, C₆₋₁₂ aryl, and 5 to 12-membered heterocyclyl;
R¹⁴ is a C₁₋₁₂ alkyl or a C₃₋₁₂ cycloalkyl;
R¹⁵ and R¹⁶ each independently is selected from C₁₋₁₂ alkyl, C₆₋₁₂ aryl, and 5 to 12-membered heterocyclyl; or R¹⁵ and R¹⁶ together with the carbon atoms to which they are attached form a C₆₋₁₂ aryl or a 5 to 12-membered heterocyclyl; and
R¹⁷, R¹⁸, and R¹⁹ each independently is a C₁₋₁₂ alkyl or a C₃₋₁₂ cycloalkyl; or
R¹⁸ and R¹⁹ together with the nitrogen atom to which they are attached form a 5 to 12-membered heterocyclyl and (i) R¹⁷ is a C₁₋₁₂ alkyl or a C₃₋₁₂ cycloalkyl, or (ii) R¹⁷ and at least one atom of L³, if present, together with the nitrogen atom to which L³ and R¹⁷ are attached form a 5- to 12-membered heterocyclyl; or
R¹⁷, R¹⁸, and R¹⁹ together with the nitrogen atom to which they are attached form a bicyclic 5 to 12-membered heterocyclyl; and
L² is selected from a C₁₋₁₂ alkylene, C₆₋₁₂ arylene, C₆₋₁₂ arylene-C₁₋₁₂ alkylene, C₁₋₁₂ alkylene-C₆₋₁₂ arylene, C₁₋₁₂ alkylene-O—C₁₋₁₂ alkylene, (O—C₁₋₁₂ alkylene)ₘ, (C₁₋₁₂ alkylene-O)ₘ, C₁₋₁₂ alkylene-NH—C₁₋₁₂ alkylene, C₁₋₁₂ alkylene-N(C₁₋₁₂ alkyl)-C₁₋₁₂ alkylene, (NH—C₁₋₁₂ alkylene)ₘ, and (C₁₋₁₂ alkylene-NH) m;
L³ is selected from a C₁₋₁₂ alkylene, C₆₋₁₂ arylene, C₆₋₁₂ arylene-C₁₋₁₂ alkylene, C₁₋₁₂ alkylene-C₆₋₁₂ arylene, C₁₋₁₂ alkylene-O—C₁₋₁₂ alkylene, (O—C₁₋₁₂ alkylene) n, (C₁₋₁₂ alkylene-O) n, C₁₋₁₂ alkylene-NH—C₁₋₁₂ alkylene, C₁₋₁₂ alkylene-N(C₁₋₁₂ alkyl)-C₁₋₁₂ alkylene, (NH—C₁₋₁₂ alkylene) n, (C₁₋₁₂ alkylene-NH) n, and a bond;
m is an integer between 1 and 6;
n is an integer between 1 and 6;
b is 1 or 2;
Y² is selected from —C(═O)—, O, S, NH, N(C₁₋₁₂ alkyl), and a bond;
X⁻ is selected from F⁻, Cl⁻, Br⁻, OH⁻, NO⁻₂, CN⁻, HCO₃⁻, CO₃²⁻, PF⁻, BF₄⁻, a C₁₋₁₂ carboxylate and a C₁₋₁₂ alkoxide;
provided that W is C₁₋₁₂ alkyl only when V is a cationic moiety, and wherein:
each C₁₋₁₂ alkylene, C₆₋₁₂ arylene, C₅₋₁₂ cycloalkylene, 5 to 16-membered heterocyclylene, C₁₋₁₂ alkyl, C₃₋₁₂ cycloalkyl, C₅₋₁₂ cycloalkyl, C₆₋₁₂ aryl, and 5 to 12-membered heterocyclyl is independently optionally substituted with 1 to 6 substituents independently selected from the group consisting of F, Cl, Br, OH, NH₂, oxo, a C₁₋₁₂ alkyl, C₆₋₁₂ aryl, C₁₋₁₂ haloalkyl, C₁₋₁₂ alkoxy, C₆₋₁₂ aryl, C₆₋₁₂ aryloxy, NH(C₁₋₁₂ alkyl), N(C₁₋₁₂ alkyl)₂, C(O)O(C₁₋₁₂ alkyl), and C(O)NH(C₁₋₁₂ alkyl).

In a third embodiment the invention is a cross-linked polymer, comprising: a plurality of first repeat units selected from cross-linking moieties represented by structural formula (IIa) or structural formula (IIb):

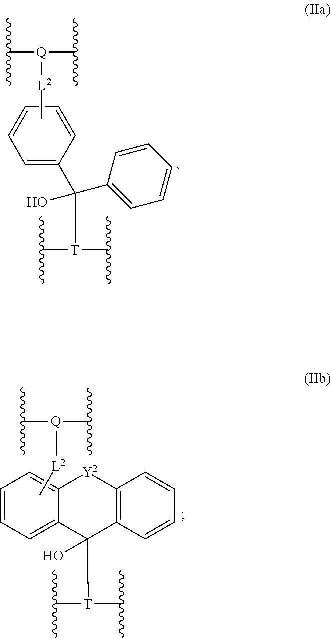

a plurality of second repeat units represented by structural formula (III):

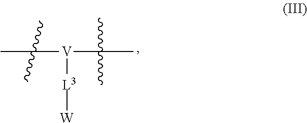

wherein:
[symbol] is a point of attachment to adjacent repeat units of the polymer;
Q is a moiety represented by one of the following structural formulas:

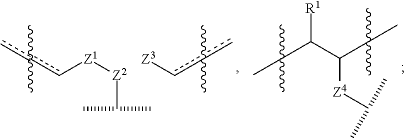

V is a moiety represented by one of the following structural formulas:

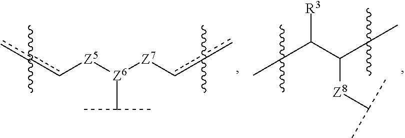

W is a C₁₋₁₂ alkyl or a moiety represented by one of the structural formulas selected from:

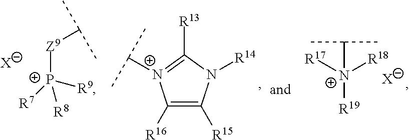

T, for each occurrence independently, is a C₂₋₈ alkylene,
[symbol] is a point of attachment to adjacent repeat units of the polymer;
[symbol] is a point of attachment to L²;
[symbol] is a point of attachment to L³
and further wherein:
[symbol] is a double bond or a single bond;
Z¹, Z³, Z⁵, and Z⁷ each independently is a C₁₋₃ alkylene or a bond;
Z² is selected from —CHR⁵—, a C₅₋₁₂ cycloalkylene, and a 5 to 16-membered heterocyclylene;
Z⁴ is selected from a bond, a C₆₋₁₂ arylene, and CR², in which case R¹ and R² together with the carbon atoms to which they are attached form a C₅₋₁₂ cycloalkyl or a 5 to 12-membered heterocyclyl;
Z⁶ is selected from —CHR⁶—, a C₅₋₁₂ cycloalkylene, and a 5 to 16-membered heterocyclylene;
Z⁸ is selected from a bond, a C₆₋₁₂ arylene, and CR⁴, in which case R³ and R⁴ together with the carbon atoms to which they are attached form a C₅₋₁₂ cycloalkyl or a 5 to 12 membered heterocyclyl;
Z⁹ is NR¹⁰ or a bond;
R¹, if present, and R³, if present, each independently is selected from H, a C₁₋₁₂ alkyl, and C₆₋₁₂ aryl;
R⁵ and R⁶ each independently is H or a C₁₋₁₂ alkyl;
R⁷, R⁸, and R⁹ each independently is selected from NR¹¹R¹², a C₆₋₁₂ aryl, and 5 to 12-membered heterocyclyl;
R¹⁰ is a C₁₋₁₂ alkyl;
R¹¹ and R¹² each independently is C₁₋₁₂ alkyl or a C₃₋₁₂ cycloalkyl, or R¹¹ and R¹² together with the nitrogen atom to which they are attached form a 5 to 12-membered heterocyclyl;
R¹³ is selected from a C₁₋₁₂ alkyl, C₆₋₁₂ aryl, and 5 to 12-membered heterocyclyl;
R¹⁴ is a C₁₋₁₂ alkyl or a C₃₋₁₂ cycloalkyl;
R¹⁵ and R¹⁶ each independently is selected from C₁₋₁₂ alkyl, C₆₋₁₂ aryl, and 5 to 12-membered heterocyclyl; or R¹⁵ and R¹⁶ together with the carbon atoms to which they are attached form a C₆₋₁₂ aryl or a 5 to 12-membered heterocyclyl; and
R¹⁷, R¹⁸, and R¹⁹ each independently is a C₁₋₁₂ alkyl or a C₃₋₁₂ cycloalkyl; or
R¹⁸ and R¹⁹ together with the nitrogen atom to which they are attached form a 5 to 12-membered heterocyclyl and (i) R¹⁷ is a C₁₋₁₂ alkyl or a C₃₋₁₂ cycloalkyl, or (ii) R¹⁷ and at least one atom of L³, if present, together with the nitrogen atom to which L³ and R¹⁷ are attached form a 5- to 12-membered heterocyclyl; or
R¹⁷, R¹⁸, and R¹⁹ together with the nitrogen atom to which they are attached form a bicyclic 5 to 12-membered heterocyclyl; and
L² is selected from a C₁₋₁₂ alkylene, C₆₋₁₂ arylene, C₆₋₁₂ arylene-C₁₋₁₂ alkylene, C₁₋₁₂ alkylene-C₆₋₁₂ arylene, C₁₋₁₂ alkylene-O—C₁₋₁₂ alkylene, (O—C₁₋₁₂ alkylene) m, (C₁₋₁₂ alkylene-O) m, C₁₋₁₂ alkylene-NH—C₁₋₁₂ alkylene, C₁₋₁₂ alkylene-N(C₁₋₁₂ alkyl)-C₁₋₁₂ alkylene, (NH—C₁₋₁₂ alkylene) m, and (C₁₋₁₂ alkylene-NH) m;
L³ is selected from a C₁₋₁₂ alkylene, C₆₋₁₂ arylene, C₆₋₁₂ arylene-C₁₋₁₂ alkylene, C₁₋₁₂ alkylene-C₆₋₁₂ arylene, C₁₋₁₂ alkylene-O—C₁₋₁₂ alkylene, (O—C₁₋₁₂ alkylene) n, (C₁₋₁₂ alkylene-O) n, C₁₋₁₂ alkylene-NH—C₁₋₁₂ alkylene, C₁₋₁₂ alkylene-N(C₁₋₁₂ alkyl)-C₁₋₁₂ alkylene, (NH—C₁₋₁₂ alkylene) n, (C₁₋₁₂ alkylene-NH) n, and a bond;
m is an integer between 1 and 6;
n is an integer between 1 and 6;
Y² is selected from —C(═O)—, O, S, NH, N(C₁₋₁₂ alkyl), and a bond;
X⁻ is selected from F⁻, Cl⁻, Br⁻, OH⁻, NO⁻₂, CN⁻, HCO₃⁻, CO₃²⁻, PF₆⁻, BF₄⁻, a C₁₋₁₂ carboxylate and a C₁₋₁₂ alkoxide;
provided that W is C₁₋₁₂ alkyl only when V is a cationic moiety, and wherein:
each C₁₋₁₂ alkylene, C₆₋₁₂ arylene, C₅₋₁₂ cycloalkylene, 5 to 16-membered heterocyclylene, C₁₋₁₂ alkyl, C₃₋₁₂ cycloalkyl, C₅₋₁₂ cycloalkyl, C₆₋₁₂ aryl, and 5 to 12-membered heterocyclyl is independently optionally substituted with 1 to 6 substituents independently selected from the group consisting of F, Cl, Br, OH, NH₂, oxo, a C₁₋₁₂ alkyl, C₆₋₁₂ aryl, C₁₋₁₂ haloalkyl, C₁₋₁₂ alkoxy, C₆₋₁₂ aryl, C₆₋₁₂ aryloxy, NH(C₁₋₁₂ alkyl), N(C₁₋₁₂ alkyl)₂, C(O)O(C₁₋₁₂ alkyl), and C(O)NH(C₁₋₁₂ alkyl).

In a fourth embodiment, the invention is a composite material, comprising a reinforcement material and a polymer described herein with respect to the second embodiment and various aspects thereof, or a cross-linked polymer described herein with respect to the third embodiment and various aspects thereof.

In a fifth embodiment, the invention is a membrane, comprising a film of the polymer described herein with respect to the second embodiment and various aspects thereof, the cross-linked polymer described herein with respect to the third embodiment and various aspects thereof; or the composite material described herein with respect to the fourth embodiment and various aspects thereof.

In a sixth embodiment, the invention is a membrane electrode assembly, comprising a membrane described herein with respect to the fifth embodiment and various aspects thereof and an electrode.

In a seventh embodiment, the invention is an electrochemical device, comprising a membrane electrode assembly described herein with respect to the sixth embodiment and various aspects thereof and a current collector.

### BRIEF DESCRIPTION OF THE DRAWINGS

FIG. 1 shows structural formulas of certain Tetrakis®-BXL polymers comprising Tetrakis® cations with various patterns of substitution in the phosphonium cation.

FIG. 2 shows a matrix demonstrating Tetrakis®-BXL polymer compositions for unsupported AEMs containing phosphonium cations with cyclohexyl, methyl substitution.

FIG. 3 shows a plot demonstrating through-plane hydroxide conductivity and area surface resistance of an AEM as a function of polymer loading (polymer loading is plotted on the x axis).

FIG. 4 shows a plot demonstrating through-plane hydroxide conductivity and area surface resistance of an AEM as a function of polymer incorporation method.

FIG. 5 shows a plot of through-plane hydroxide conductivity for various Tetrakis®-BXL rAEMs supported on polypropylene (PP) (Celgard) at room temperature.

FIG. 6 shows a plot of area surface resistance conductivity for various Tetrakis®-BXL rAEMs supported on PP (Celgard) at room temperature.

FIG. 7 shows a plot of through-plane hydroxide conductivity for various Tetrakis®-BXL rAEMs supported on PP (Celgard) at 80° C.

FIG. 8 shows a plot of area surface resistance conductivity for various Tetrakis®-BXL rAEMs supported on PP (Celgard) at 80° C.

### DETAILED DESCRIPTION

Developing anion exchange membranes (AEMs) that contain durable polymer backbones and cationic groups is required to commercialize fuel cells, electrolyzers, redox flow batteries, water purifiers and other electrochemical devices. Alkaline systems hold several benefits over acidic counterparts, particularly the fabrication of devices with less expensive electrodes and bipolar plates and longer lifetimes. Alkaline electrochemical devices are an exciting alternative to proton exchange membrane (PEM) devices because at elevated pH, oxygen reduction is more facile and lower overpotentials are required, and allowing metals other than platinum to be used as electrocatalysts.

Disclosed herein are anion exchange ionomers (AEI) and AEMs comprising said AEIs displaying desirable chemical durability under harsh alkaline environments, ability to absorb water, and low aqueous solubility. The disclosed AEMs demonstrate reduced absorption of water at high temperatures compared to state of the art materials. The AEMs maintain high ionic conductivity and low resistance without loss of mechanical properties.

### I. Polymers of the Invention Generally

In some embodiments, the AEIs can be cross-linked to improve the performance of the materials. Presented in the below paragraphs are exemplary components (e.g., backbones, repeat units, cationic moieties, or cross-linkable moieties) of cross-linkable AEIs. An AEI can comprise any combination of the components disclosed below.

### 1. Cross-Linkable Moieties

The AEIs can be cross-linked, for example, by introducing cross-linkable pendants into some of the repeat units of the polymer, for example, type II photoinitiators such as benzophenone, camphorquinone, isopropylthioxanthone, and thioxanthone (see Allushi et al., Polymer Chemistry, 2017, 8, 1972-1977). Type I photoinitiators such as dimethoxyphenylacetophenone, α-hydroxyacetophenone, α-aminoacetophenone, benzoylphosphinoxide, bisbenzoylphosphinoxide, can also be introduced into some of the repeat units of the polymer.

### 2. Polymer Backbones

A cross-linkable pendant can be incorporated in the polymer by connecting it to a repeat unit. For example, the following repeat units or polymer backbones can be functionalized with cross-linkable pendants:

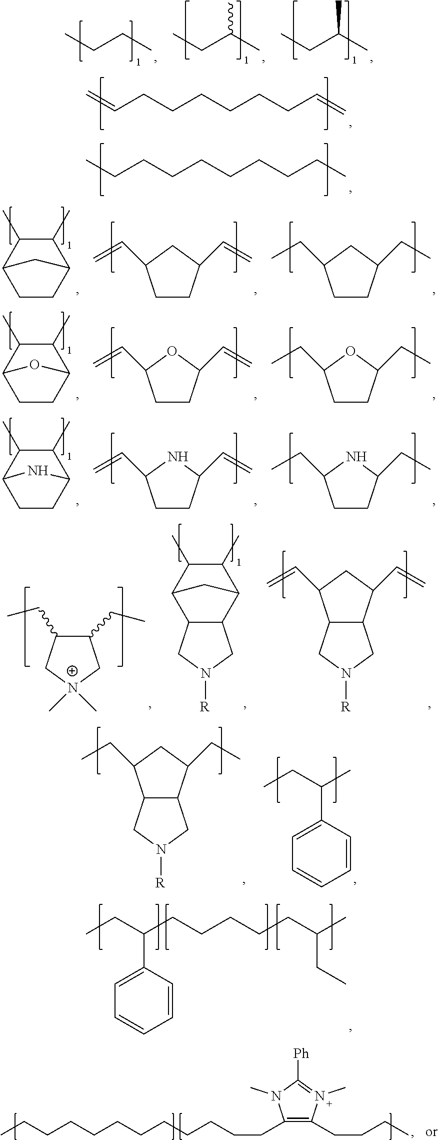

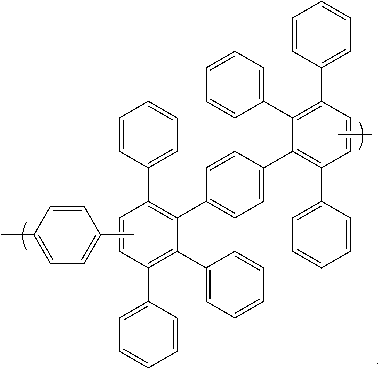

### 3. Cationic Moieties.

The AEIs can comprise various cationic moieties. For example, the cationic moieties can be incorporated in an AEI as pendants linked to the backbone of the polymer. Additionally or alternatively, the backbone of the polymer can comprise cationic groups. For example, the following cationic moieties can be incorporated in an AEI (indicates the point of attachment of the cationic moiety to the backbone or to a linker connected to the backbone):

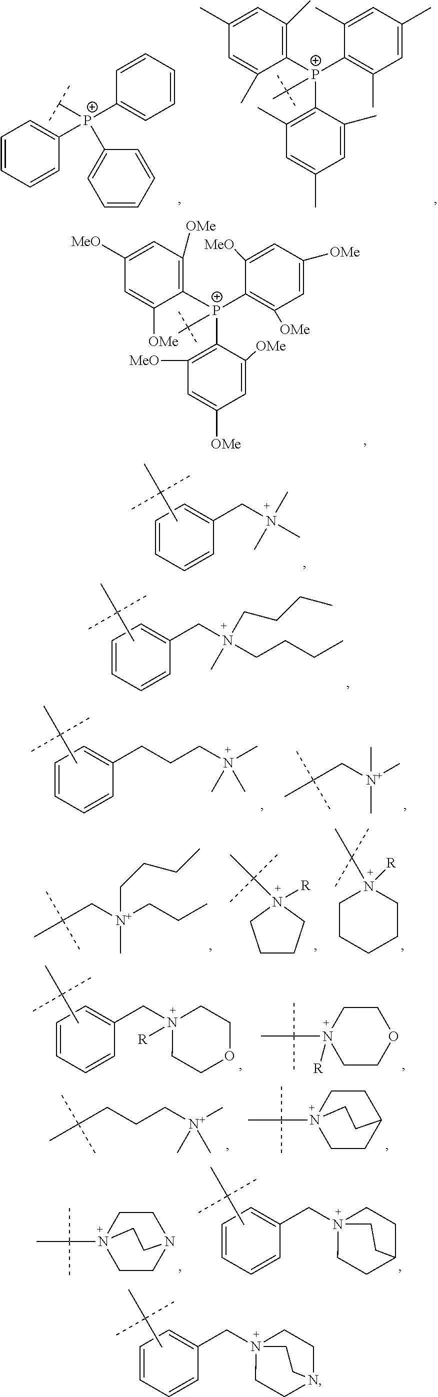

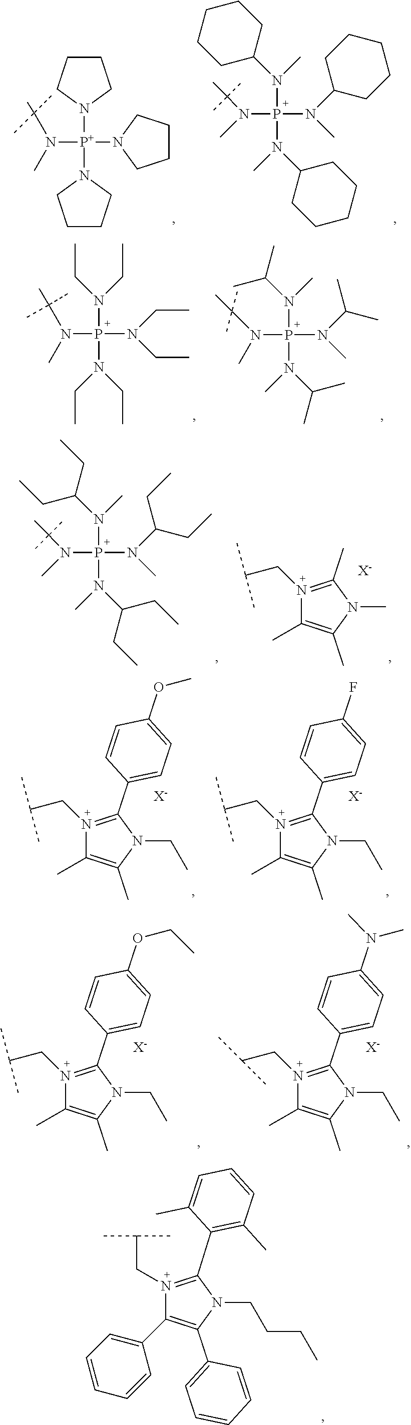

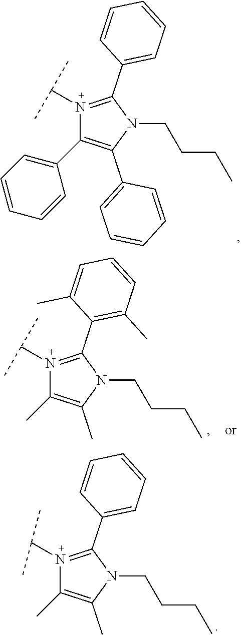

### 4. Repeat Units

The AEIs can comprise the following combinations of repeat units:

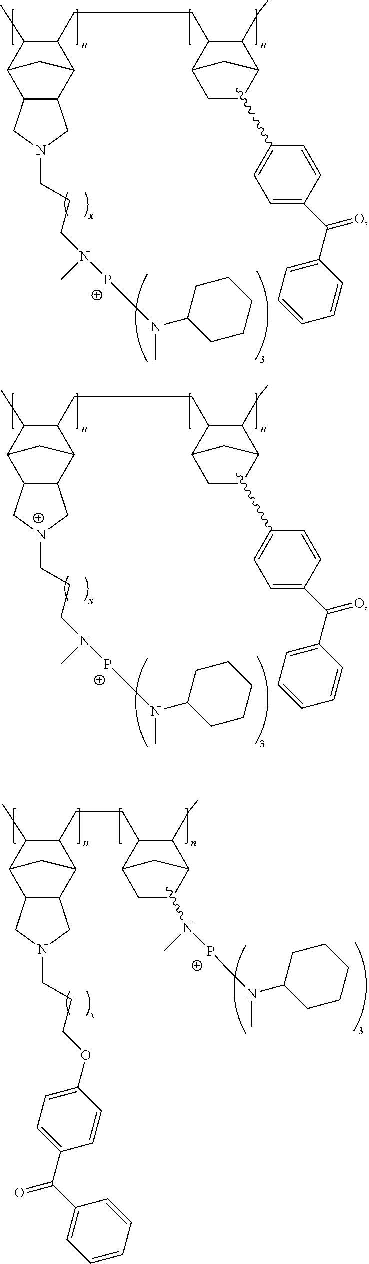

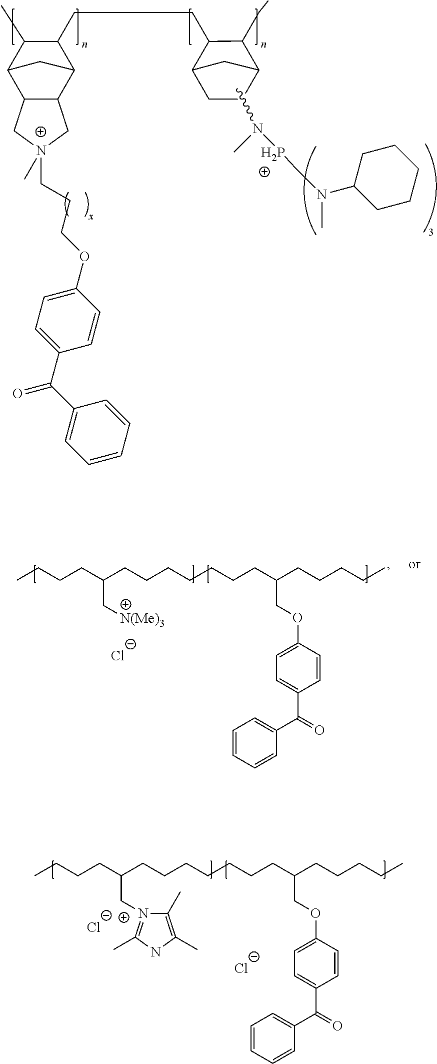

### II. Certain Examples of the Polymers of the Present Invention

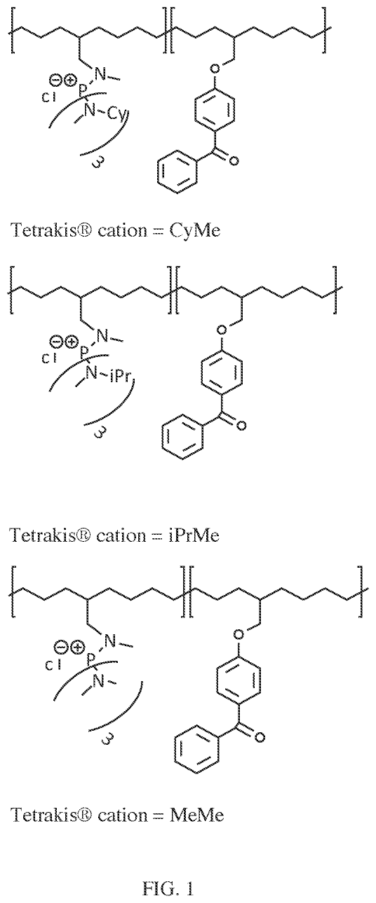

Examples of the AEIs described above were further investigated. Disclosed herein is a class of polymers comprising Tetrakis® cations and benzophenone, the Tetrakis®-BXL series (FIG. 1), that can be cross-linked using UV light. The term Tetrakis® refers to a cation of the following structural formula:

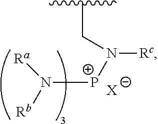

wherein [symbol] is a point of attachment to the polymer or a linker connected to the polymer, and R^{a}, R^{b}, and R^{c} each independently is an alkyl or a cycloalkyl.

Using photo-catalysis to crosslink the polymers is advantageous because the polymers can be synthesized and fabricated in any form factor needed (films, powders, solutions) prior to irreversible cross-linking. UV curing of coatings is common in polymer manufacturing and simplifies the processing for production at larger scale. This is preferred over methods that crosslink in-situ or by chemical soaking after fabrication because these methods are difficult to translate in large scale manufacturing.

Tetrakis®-containing polymer are labelled as follows:

T-xx-yyy refers to a polyelectrolyte represented by the following structural formula:

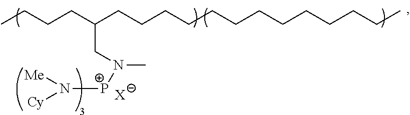

comprising xx mol. % of the cationic repeat unit and having MWn of yyy,000 g/mol.

T-xx-yyy-BXLz refers to a polyelectrolyte represented by the following structural formula:

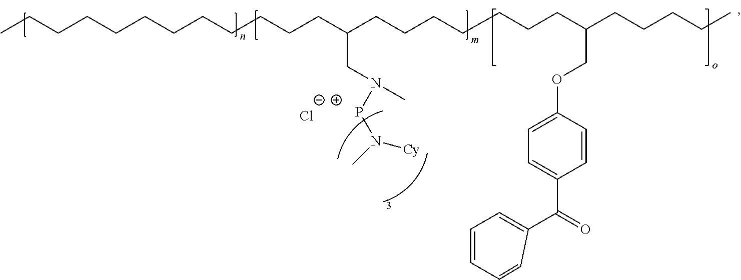

comprising xx mol. % of the cationic repeat unit, z mol. % of the benzophenone-containing repeat unit, and having MWn of yyy,000 g/mol.
IIa. Tetrakis®-BXL AEIs and Free-Standing Tetrakis®-BXL AEMs.

An example of the Tetrakis®-BXL AEI is T-28-120-BXL2, which does not require purification, has lower aqueous solubility than previous benzophenone-free Tetrakis®-containing AEIs, and maintains high water uptake at 80° C. The benzophenone-containing (BXL) polymers are highly soluble and processable in organic solvents prior to UV curing. After cross-linking the new AEI can be formulated as an insoluble powder dispersion.

Unsupported cross-linked BXL-containing AEMs were prepared co-polymerizing by Tetrakis®-functionalized cyclooctene, benzophenone-functionalized cyclooctene, and cyclooctene. The benchmark non-crosslinked Tetrakis® prototype is T-23-300, containing 23% cation and a molecular weight of 300,000 g/mol. Initially, a series of polymers were prepared that explored cation content, with BXL content of 15% and a similar molecular weight to T-23-300. The resulting polymers were difficult to handle, curling excessively when manipulated and hydrated. The poor handling resulted in irreproducible polymer properties due to 1) inconsistencies in the films or 2) unavoidable measurement errors. It was discovered that the combination of high molecular weight and high benzophenone-crosslink (BXL) content (>15%) is not desirable for free-standing AEMs. As such, a series of polymers with lower molecular weight and crosslinking density were prepared and analyzed. T-28-120-BXL2 and T-28-120-BXL5 were identified as promising AEIs for unsupported, cross-linked AEMs and T-28-120-BXL5. Additionally, the Tetrakis®-BXL AEMs and Tetrakis® AEMs were compared to commercially available AEMs. Presented in Table 1 are structural characteristics of Tetrakis® and Tetrakis®-BXL AEMs and commercially available AEMs.

TABLE 1
Structural features of evaluated materials.

| Tetrakis ® | | | | | |
|---|---|---|---|---|---|
| AEMs: T-25-360 | | | | | |
| T-23-300 | | | | | |
| T-28-120-BXL5 | | | | | |
| AEIs: T-28-120 | | Fumapem ® | Sustainion ® | | |
| Tₚ-23-275* | Pention | AEM: FAA-3-50 | AEM: X37-50 | Xion Durion ™ | Fumasep ® |
| T-28-120-BXL2 | AEM-72-30-15% | AEI: FAA-SOLUT-10 | AEI: XB-7 | AEM: LMW-215-30 | AEM: FAS-50 |
| Cation: Dialkyl(amino) | Cation: Long-chain | Cation: Ammonium | Cation: Benzyl | Cation: Long-chain | Cation: Ammonium |
| phosphonium | alkyl ammonium | Backbone: | imidazolium | alkyl ammonium | Backbone: |
| Backbone: Linear, | Backbone: | Polyaromatic | Backbone: | Backbone: | Hydrocarbon |
| aliphatic, | Polynorbornene | Slightly | Polystyrene- | Poly(terphenylene) | Slightly |
| hydrocarbon | Cross-linked | cross-linked | based | with CF₃ groups | cross-linked |
| Cross-linked | PTFE Reinforced | Non-Reinforced | Cross-linked | Cross-linked | Non-Reinforced |
| (BXL series) | Thickness: | Thickness: | Non-Reinforced | PTFE Reinforced | Thickness: |
| Non-Cross-linked | 30 μm | 45-50 μm | Thickness: | Thickness: | 45-55 μm |
| (All others) | | | 50 μm | 30 μm | |
| Non-Reinforced | | | | | |
| Thickness: | | | | | |
| 45-55 μm | | | | | |

*Tₚ-23-275 refers to purified T-23-275.

### 1. Comparison of Non-Crosslinked and Cross-Linked Tetrakis®-Containing Ionomers.

Three ionomers, non-cross-linked T-28-120 and Tₚ-23-275, and cross-linked T-28-120-BXL2 have been examined with respect to a number of parameters essential for the AEM performance. Table 2 summarizes the characterization data.

TABLE 2
Ex-Situ Characterization of Tetrakis ®-containing ionomers.

| AEI Ex-Situ | | | |
|---|---|---|---|
| Characterization | T-28-120 | Tₚ-23-275 | T-28-120-BXL2 |
| Organic Solubility | 96.1 ± 1.6 | 80.4 | >99% [not X-linked] |
| | [n-propanol - 5 wt %] | [H₂O:n-PrOH:DMF, | 14.9 ± 8.3 [X-linked] |
| | | 1:2:7] | [n-propanol - 5 wt %] |
| Aqueous Solubility | <2% | <2% | 4.1 ± 1.2 |
| @ RT and @ 80° C. | 45.4 ± 1.1 | 9.10 ± 2.1 | 15.3 ± 7.3 |
| Ion Exchange Capacity | 1.15 ± 0.06 | 1.01 ± 0.06 | 1.20 ± 0.20 |
| (IEC) | | | |
| Water Uptake | 147 ± 27 | 95.0 ± 14.4 | 117 ± 10 |
| @ RT and @ 80° C. | Soluble | 281 ± 6 | 347 ± 65 |
| X-Y Swelling | 31.0 ± 7.7 | 18.0 ± 1.2 | 29.9 ± 2.3 |
| @ RT and @ 80° C. | Soluble | 43.7 ± 2.5 | 62.4 ± 6.1 |
| Z Swelling | 44.1 ± 11.2 | 24.1 ± 5.0 | 2.9 ± 21 |
| @ RT and @ 80° C. | Soluble | 78.8 ± 16.3 | 47.0 ± 24.2 |
| Alkaline Stability | N/A | <10% loss (200 h) | <2% loss (2000 h) |
| via IEC* | | i = 1.01 ± 0.06 | i = 1.10 ± 0.1 |
| | | f = 1.03 ± 0.03 | f = 1.20 ± 0.2 |
| Alkaline Stability | N/A | N/A | <10% loss (200 h) |
| via Conductivity* | | | i = 27.5 ± 2.0 |
| | | | f = 24.2 ± 1.2 |

*i—initial; f—final

Tₚ-23-275 and T-28-120-BXL2 both have significantly less aqueous solubility and higher water uptake compared to the benchmark T-28-120.

T-28-120-BXL2 was shown to be a versatile AEI. It can be formulated as a 5 wt % solution in n-propanol before crosslinking. The material can be manipulated as an ionomer solution and in catalyst inks, with photo-crosslinking at the very end of electrode fabrication. Moreover, T-28-120-BXL2 can also be formulated as an insoluble powder dispersion in isopropanol if that methodology is desired in electrode fabrication.

### 2. Comparison of Cross-Linked Tetrakis®-Containing Ionomers to Commercially Available Ionomers.

Cross-linked AEI T-28-120-BXL2 was further compared to commercially available ionomers from Fumatech (Fumion FAA-3-50) and Dioxide Materials (Sustainion® XB-7). Table 3 summarizes the characterization data for T-28-120-BXL2 and commercial AEIs.

TABLE 3
Comparison of T-28-120-BXL2 to Commercial AEIs.

| AEI Ex-Situ | | Fumion | Sustainion ® |
|---|---|---|---|
| Characterization | T-28-120-BXL2 | FAA-3-SOLUT-10 | XB-7 |
| Organic Solubility | 14.9 ± 8.3 | 95.9 ± 1.3 | 90.6 ± 3.2 |
| | [n-propanol - 5 wt %] | [10.9 wt %] | [4.5 wt %] |
| Aqueous Solubility | 4.1 ± 1.2 | 2.10 ± 0.95 | 30.8 ± 10.6 |
| @ RT and @ 80° C. | 15.3 ± 7.3 | 7.50 ± 0.20 | 48.6 ± 17.7 |
| Ion Exchange Capacity | 1.20 ± 0.20 | 1.15 ± 0.09 | 2.47 ± 0.44 |
| (IEC) | | | |
| Water Uptake | 117 ± 10 | 40.5 ± 10.8 | 86.3 ± 31.1 |
| @ RT and @ 80° C. | 347 ± 65 | 209 ± 35 | Mechanical Failure |
| X-Y Swelling | 29.9 ± 2.3 | 19.9 ± 1.0 | Mechanical Failure |
| @ RT and @ 80° C. | 62.4 ± 6.1 | 43.7 ± 2.5 | Mechanical Failure |
| Z Swelling | 2.9 ± 21 | 18.2 ± 5.7 | 36.7 ± 20.0 |
| @ RT and @ 80° C. | 47.0 ± 24.2 | 60.7 ± 10.6 | Mechanical Failure |
| Alkaline Stability | <2% loss (2000 h) | N/A | N/A |
| via IEC* | i = 1.10 ± 0.1 | | |
| | f = 1.20 ± 0.2 | | |
| Alkaline Stability | <10% loss (200 h) | N/A | N/A |
| via Conductivity* | i = 27.5 ± 2.0 | | |
| | f = 24.2 ± 1.2 | | |

*i—initial; f—final

The data in Table 3 indicate that T-28-120-BXL2 outperforms both Fumion and Sustainion ionomers.

### 3. Comparison of Non-Crosslinked and Cross-Linked Tetrakis®-Containing AEMs.

Cross-linked AEM T-28-120-BXL5 was compared to Tetrakis®-containing non-cross-linked membranes, T-25-360 and T-23-300. The comparison data are presented in Table 4.

TABLE 4
Comparison of AEMs: T-28-120-BXL5 to T-25-300 and T-25-360.

| AEM Ex-Situ Characterization | T-25-360 | T-23-300 | T-28-120_BXL5 |
|---|---|---|---|
| In-Plane Conductivity, | 33.3 ± 2.9 | 28.7 ± 4.3 | 25.6 ± 1.2 |
| Hydroxide @ RT and @ 80° C. | 29.6 ± 6.9 | 28 ± 3.7 | 59.9 ± 8.3 |
| Through-Plane Conductivity, | 26.4 ± 1.5 | 26.4 ± 1.5 | 27.6 ± 2.1 |
| Hydroxide @ RT and @ 80° C. | N/A | 80.7 ± 8.4 | 72.9 ± 7.3 |
| Areal Specific Resistance, | 0.23 ± 0.02 | 0.21 ± 0.02 | 0.19 ± 0.01 |
| Hydroxide @ RT and @ 80° C. | N/A | 0.10 ± 0.01 | 0.05 ± 0.01 |
| Ion Exchange Capacity (IEC) | 1.11 ± 0.23 | 1.04 ± 0.06 | 1.60 ± 0.4 |
| Water Uptake | 93.1 ± 4.1 | 99.0 ± 21.9 | 56.6 ± 9.7 |
| @ RT and @ 80° C. | 1040 ± 222 | 317 ± 78 | 96.9 ± 13.3 |
| X-Y Swelling | 28.1 ± 1.0 | 21.4 ± 1.1 | 14.5 ± 5.0 |
| @ RT and @ 80° C. | 101 ± 9.0 | 48.7 ± 2.8 | 16.9 ± 5.2 |
| Z Swelling | 19.4 ± 5.0 | 11.6 ± 3.7 | <2% |
| @ RT and @ 80° C. | 102 ± 25 | 60.2 ± 23.0 | <2% |
| Mechanical Stress @ Break | 0.862 ± 0.594 | 6.98 ± 2.45 | 20.3 ± 1.4 |
| Mechanical Strain @ Break | 299 ± 81 | 1140 ± 306 | 165 ± 11 |
| Humidity Stability Factor (HSF) | 3.0 | 23.4 | 9.9 |
| Alkaline Stability via IEC* | <5% loss (200 h) | <5% loss (200 h) | <2% loss (2000 h) |
| | i = 1.10 ± 0.23 | i = 1.04 ± 0.06 | i = 0.90 ± 0.10 |
| | f = 2.70 ± 0.50 | f = 1.11 ± 0.13 | f = 1.49 ± 0.26 |
| Alkaline Stability | <5% loss (200 h) | <5% loss (200 h) | <5% loss (200 h) |
| via Conductivity* | i = 33.3 ± 2.9 | i = 28.7 ± 4.3 | i = 25.6 ± 1.2 |
| | f = 38.9 ± 3.0 | f = 27.9 ± 2.1 | f = 24.3 ± 3.3 |

*i—initial; f—final

The data in Table 4 demonstrate that the cross-linked version of the Tetrakis® AEM (T-28-120-BXL5) showed reduced membrane swelling and water uptake and allowed for high conductivity and low resistance at room temperature and 80° C. The handling properties of T-28-120-BXL5 were excellent at room temperature and at 80° C., even at low thickness (30 microns). The AEM appeared very mechanically tough, without being brittle.

### 4. Comparison of Cross-Linked Tetrakis®-Containing AEMs to Commercially Available AEMs.

Cross-linked AEM, T-28-120-BXL5, was further compared to commercially available membranes from Xergy (Xion Durion™ LMW-215-30 & Pention 215-72-30), Fumatech (FAA-3-50 & FAS-50), Dioxide Materials (Sustainion® X37-50). The comparison data are presented in Table 5. The structural features of the commercial AEMs are listed in Table 1.

TABLE 5
Comparison of cross-linked AEM T-28-120-BXL5 to commercial AEMs.

| AEM Ex-Situ | | Pention | Fumapem | Sustainion | Xion Durion |
|---|---|---|---|---|---|
| Characterization | T-28-120_BXL5 | AEM-72-30-15 | FAA-3-50 | X37-50 | LMW-215-30 |
| In-Plane Conductivity, OH⁻ @ | 25.6 ± 1.2 | 29.8 ± 2.8 | 40.0 ± 2.9 | 37.7 ± 1.5 | 39.2 ± 4.3 |
| RT and @, 80° C. | 59.9 ± 8.3 | 109 ± 14 | 59.9 ± 4.8 | 16.2 ± 3.2 | 78.6 ± 23.3 |
| Through-Plane Conductivity, OH⁻ @ | 27.6 ± 2.1 | 23.4 ± 2.8 | 7.9 ± 1.1 | 38.5 ± 3.3 | N/A |
| RT and @ 80° C. | 72.9 ± 7.3 | 40.3 ± 5.7 | N/A | N/A | N/A |
| Areal Specific Resistance, OH⁻ @ | 0.19 ± 0.01 | 0.17 ± 0.02 | 0.78 ± 0.09 | 0.26 ± 0.02 | N/A |
| RT and @ 80° C. | 0.08 ± 0.01 | 0.11 ± 0.02 | N/A | N/A | N/A |
| Ion Exchange Capacity (IEC) | 1.60 ± 0.40 | 2.79 ± 0.40 | 2.00 ± 0.10 | 3.00 ± 0.20 | 1.54 ± 0.06 |
| Water Uptake | 56.6 ± 9.7 | 65.3 ± 19.9 | 24.5 ± 4.1 | 170 ± 4 | 133 ± 31 |
| @ RT and @80° C. | 96.9 ± 13.3 | 56.6 ± 4.9 | 302 ± 55 | 581 ± 116 | 500 ± 47 |
| X-Y Swelling | 14.5 ± 5.0 | 8.5 ± 12.2 | 14.1 ± 2.2 | 62.5 ± 1.4 | 1.4 ± 1.3 |
| @ RT and @ 80° C. | 16.9 ± 5.2 | 8.8 ± 15.9 | 99.3 ± 6.1 | 98.4 ± 7.7 | N/A |
| Z Swelling | <2% | 9.3 ± 3.6 | 11.9 ± 2.6 | 59.3 ± 2.7 | 126 ± 30 |
| @ RT and @ 80° C. | <2% | 18.8 ± 3.0 | 77.3 ± 8.9 | 81.7 ± 21.2 | 448 ± 23 |
| Mechanical Stress @ Break | 20.3 ± 1.4 | 27.7 ± 3.4 | 13.2 ± 4.6 | 0.32 ± 0.16 | 38.2 ± 2.6 |
| Mechanical Strain @ Break | 165 ± 11 | 5.1 ± 1.6 | 13.5 ± 4.1 | 33.9 ± 9.4 | 6.6 ± 1.1 |
| Humidity Stability Factor (HSF) | 9.9 | 0.3 | 0.1 | 0.3 | 0.07 |
| Alkaline Stability via IEC* | <2% loss (2000 h) | N/A | 64% loss (500 h) | 54% loss (500 h) | N/A |
| | i = 0.90 ± 0.10 | | i = 1.52 ± 0.15 | i = 2.82 ± 0.95 | |
| | f = 1.49 ± 0.26 | | f ± 0.98 ± 0.35 | f = 1.52 ± 0.23 | |
| Alkaline Stability | <5% loss (200 h) | <5% loss (200 h) | 93% loss (500 h) | 50% loss (500 h) | N/A |
| via Conductivity* | i = 25.6 ± 1.2 | i = 29.8 ± 2.8 | i = 40.0 ± 2.9 | i = 37.7 ± 1.5 | |
| | f = 24.3 ± 3.3 | f = 44.5 ± 2.2 | f = 2.78 ± 1.47 | f = 18.9 ± 1.0 | |

*i—initial; f—final

With the exception of T-28-120-BXL5 and Pention 72-30-15, the AEMs absorbed excessive amounts of water at 80° C. High water uptake and swelling in the AEMs results in lower conductivities at high temperature, in addition to softening and difficulty handling.

High water uptake at room temperature (RT) and 80° C. was observed for Xion Durion™ 215-30, which is reported to be a cross-linked and reinforced material. Pention 72-30-15 is also cross-linked and reinforced with a polytetrafluoroethylene (PTFE) support. In contrast, T-28-120-BXL5 is cross-linked, but not reinforced. The ability of T-28-120-BXL5 to achieve low swelling and high conductivity without reinforcement is an advantage for manufacturing because it requires less processing, resources and development. Moreover, Pention 72-30-15 requires a chemical crosslinking process, by which the films are soaked in an amine solution. This type of chemical crosslinking may be difficult to achieve reproducibly at scale. T-28-120-BXL5 AEM exhibits excellent in-plane and through-plane hydroxide ion conductivity, as well as low area-specific resistance (ASR) at 80° C. As such, the water uptake may be sufficiently low for excellent device performance.

IIb. Reinforced Tetrakis®-BXL AEMs (rAEMs).

The virtues of increasing AEM charge density are diminished if the polymers affinity for water is too great. Some water uptake is necessary for proper ion transport, yet too much swelling has negative impacts. It reduces the mechanical properties of unsupported AEMs, and 3D swelling lowers ionic conductivity by increasing the distance ions travel. Large changes in the dimensions of the polymer during humidity cycling increases the stress forces on membranes and is particularly problematic for fuel cell electrolytes. Moreover, AEMs can become water soluble at very high IECs. Reinforced AEMs (composites) are less susceptible to the mechanical issues, but water solubility remains a problem. Even though increasing the polymer molecular weight is easily accomplished with the disclosed polymerization procedures, further modifications are needed to inhibit aqueous solubility at optimal IEC values. Cross-linking polymers is a common way to completely prevent solubility.

To make the AEMs even more compelling, it is important to reduce overall resistance by decreasing the thickness of the electrolyte layer and increasing ionic conductivity, without sacrificing mechanical strength. Conventional methods to increase conductivity involve increasing the ion content in the polymer or IEC. This strategy is easily accomplished with the ROMP technique. However, higher ion content results in higher water uptake and excessive swelling of the polymer electrolyte, resulting in mechanical failure. Certain AEMs are pliable films that are not brittle at reduced thickness but swelling of such membranes in water at high temperatures can make them too viscoelastic. Including an additional facet to the system that simultaneously allows for higher IEC of thinner electrolytes, mechanical strength, and reduced dimensional changes in the hydrated electrolyte produces desirable AEM products.

### 1. Porous Supports

The present disclosure provides methodology of infusing the AEM material into the unoccupied spaces within various porous polymer structural supports. The structural rigidity and mechanical strength of the supports was successfully combined with the electrochemical properties of the polymer electrolytes. The resulting composites were fully characterized to analyze the level of polymer impregnation, water uptake levels, thermal characteristics and electrochemical performance. Polymer materials from several international companies were obtained, including polyethylene (PE) and polypropylene (PP).

There is an optimum amount of void space remaining in the dry AEM composite after fabrication. Essentially, the bare porous supports have a defined amount of pore volume. During fabrication, the AEM material is dissolved in a solvent that is compatible with the support and then the mixture is applied to the support to fill the pores of the support material. When the solvent is removed, the dry composite has a new void volume. The reinforced AEMs are hydrated prior to use in electrochemical devices and the polymer embedded in the support swells, once again filling much of the void space. A specific amount of void space is needed in the dry AEM to maintain high density of cations for ion transport, but also contain the right amount of space for water. The precise amount of void space will be unique to each type of polymer electrolyte and porous support combination.

BET can be used to analyze how void volume changes from the bare support and the dry composite. The primary variables to tune the void space are solvent identity and concentration of polymer in solution, as well as the method of introducing the solution to the support matrix. The solvent selected must be compatible with the support polymer and solubilize the AEM to the desired level. Often co-solvent mixtures are explored as well. The concentration must also be optimized because excessively high concentrations may prevent AEM getting into the support and not enough AEM will penetrate the support if it is too low. A rheometer can be used to characterize the viscosity of the polymer solutions and a Zetasizer to analyze the uniformity and dispersion of polymer particles. Measuring these solution properties, which impact the quantity and distribution of AEM in the support, aids in composite optimization. The ionic conductivity can be measured in conjunction with void volume to establish the link between the physical property and the electrochemical performance.

Porous polymer supports are typically designed for filtration and separation of solids, liquids and gases or to sterilize biological solutions, and are not optimized to be filled with another polymer to generate high-performing components for electrochemical devices. Generally, optimizing the specifications of supports for these applications does not provide enough overlap for the class of supports needed for composites. Therefore, it is important to develop polymer supports that are uniquely designed with composites as the end application in mind.

The first consideration to custom design a support for composites is determining what polymer material to use. PE and PP are polymers with high chemical resistance. The best thermal properties are observed with PTFE; however, it is a very expensive raw material, that is not recyclable and the processing method to fabricate porous materials from PTFE is limited to expanding. PE and PP are both significantly less expensive than PTFE, they are both recyclable and can be processed using many types of methods.

The next consideration for designing the custom support is selecting the fibers and method of fabricating the fibers into mats or sheets of material. The method can be limited for some polymers, for example PTFE can only be expanded into sheets. PE and PP films can be prepared with a variety of polymer fabrication methods. The type of fabrication has a significant impact on the morphology and alignment of the polymer strands. These features can influence how the polymer electrolyte interacts with the support and how readily it fills the voids, thus influencing composite performance. Furthermore, the mechanical properties of the support will change based on the diameter of the fibers used and how they are arranged in relation to each other, impacting the composite durability. Both features must be considered to obtain the best characteristics in the final material. The overall thickness of the support must be designed as well. Preliminary results indicate that AEMs with lower thickness also have lower resistance (A 57 m thick AEM had a resistance of 256 mΩ while a 74 m thick AEM had a resistance of 458 mΩ).

Additional features of porous supports that can be customized include pore size and porosity. Pore size simply indicates how large the average pore sizes are in a given section of support. Porosity indicates how much of the volume inside a given area is free volume, versus taken up by the support. Porosity is another way to characterize the free volume of the bare support. Both of these features will impact how the polymer electrolyte fills the voids in the support and the resulting mechanical strength of the composites.

The pore size and porosity of the supports are measured with BET, before and after filling with polymer electrolyte, to verify fabrication methodology and to support development of optimized composites. Dynamic light scattering (DLS) with a Zetasizer and rheology measurements are useful to characterize dip-coating solutions and catalyst ink formulations.

The summary of porous supports explored is provided in Table 6.

TABLE 6
PP and PE supports explored in Tetrakis ®-BXL rAEMs.

| | | | | | |
|---|---|---|---|---|---|
| PP Celgard | Gurley: 100 sec/dL | Gurley: 255 | Gurley: 230 | Gurley: 200 | Gurley: 620 |
| | Thickness: 12 μm | Thickness: 12 μm | Thickness: 16 μm | Thickness: 25 μm | Thickness: 25 μm |
| Backbone: | Porosity: 52% | Porosity: 42% | Porosity: 50% | Porosity: 55% | Porosity: 41% |
| Polypropylene | Pore Size: n/a | Pore Size: n/a | Pore Size: n/a | Pore Size: n/a | Pore Size: n/a |
| PE (J) Teijin | Gurley: unknown | | | | |
| | Thickness: 30 μm | | | | |
| Backbone: | Porosity: 85% | | | | |
| Polyethylene | Pore Size: 1 μm | | | | |
| PE (R) | Gurley: 70 sec/dL | Gurley: 70 | Gurley: 110 | | |
| | Thickness: 7 μm | Thickness: 9 μm | Thickness: 5 μm | | |
| Backbone: | Porosity: 40% | Porosity: 46% | Porosity: 33% | | |
| Polyethylene | Pore Size: n/a | Pore Size: n/a | Pore Size: n/a | | |

Gurley value refers to the time required for a specified amount of air to pass through a specified area of a separator in a battery under a specified pressure. The Gurley value reflects the tortuosity of the pores, when the porosity and thickness of the separator is fixed.

In some embodiments, the desired thinner electrolytes comprise composite AEMs by filling porous polymer supports with the AEM materials. PE and PP structural supports were used as support materials. In addition to permitting higher IEC, porous composites may provide a less tortuous path to facilitate the passage of anions through the electrolyte layer, further boosting performance, without compromising mechanics or stability. Using supports allows for a wider range of thicknesses by casting membranes into thin, composite materials.

2. Fabrication Methodology for rAEMs.

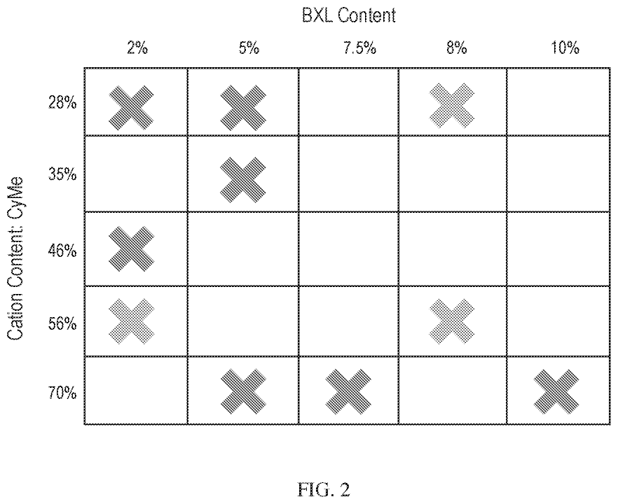

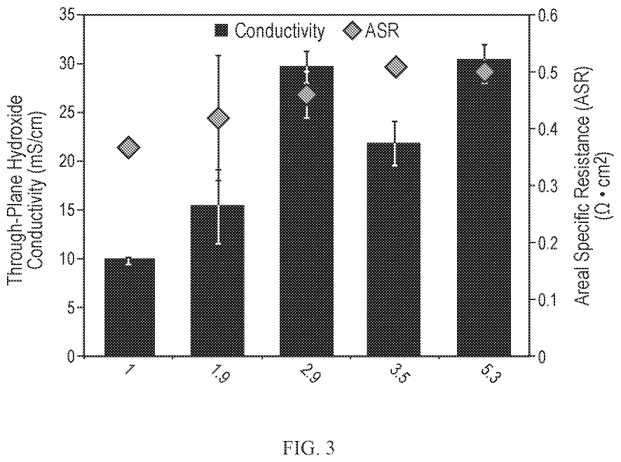

Various fabrication methods for rAEMs were explored using several types of commercially available mesoporous supports and Tetrakis®-BXL AEIs. A summary of the polymer compositions prepared is presented in FIG. 2. Two rAEMs have been prepared with polypropylene supports that have high conductivity, low ASR, low-moderate water uptake and excellent mechanical properties. Moreover, one rAEM has been prepared with a polyethylene support material that demonstrates promising conductivity and low ASR. There is an optimum value for how much polymer should be incorporated into a support material for the best rAEM properties. Experiments suggested that a void volume may enhance the properties of the rAEMs and filling the support materials completely does not produce the best rAEM. Very high polymer loadings resulted in poor handling of the materials that make them difficult to use at various conditions. Many of the commercial AEMs “curl” excessively into tight locked DNA-like strands and can't be unrolled which makes them have poor flexibility and mechanical rigidity. The data in FIG. 3 shows that conductivity and ASR of an AEM level off at high polymer loadings (x-axis).

Dip-coating, drop-casting, and spray-coating were evaluated as methods of incorporating the polymer into the support. For the drop-casting method multiple solution viscosities were explored to improve penetration into the support, both higher viscosity solutions with more polymer solids and lower viscosity solutions containing less polymer solids. However, it was discovered that this method resulted in thick coats of polymer on the surface of the support materials and did not maximize the amount of incorporated polymer. As a result, spray-coating method was selected due to its potential as a scalable technique. The spray-coat method applies polymer solution using a directional force, rather than allowing gravity to slowly facilitate incorporation. Spray-coating produces rAEMs with excellent uniformity and at much larger scales than current unsupported film. Spray coating was selected as the method of incorporating Tetrakis®-BXL polymer into a support material to create an rAEM. The method provides good incorporation by providing a gentle and consistent force that pushes the polymer into the support.

The following parameters have been identified as important for fabrication of AEMs via spray coating.

i. Percent Solids of Polymer in Casting Solution

A solution with 4 wt % polymer solids (Tetrakis®-BXL series) is low viscosity and compatible with spray coating. Several layers of polymer are easily administered with spray coating techniques, which offers many advantages over other coating methods. This advantage of the Tetrakis®-BXL is due to the following factors:
1) presence of benzophenone makes the polymer more hydrophobic in parts. When adding solvent mixture (containing water) to the BXL series of polymers, the polymers form small tight balls that result in uniform, homogenous suspensions with low viscosity. By adding a more hydrophobic component, the polymers are not prone to fully dissolving in the hydrophilic solvent, instead forming a uniform suspension;
2) Incorporation of benzophenone helps break up aggregation of cationic segments in the polymers. In a polymer without benzophenone, the cationic parts of the polymer chains strongly prefer each other and they aggregate, reducing “solubility” or increasing viscosity. Adding benzophenone hydrophobic segments breaks up the cationic aggregation, leading to lower viscosity.
   ii. Solvent Selection

A co-solvent of 2:1 water:n-propanol was identified as an effective solvent system for spray coating the Tetrakis®-BXL series of polymers onto various mesoporous substrates. The solvent system works well with PP (polypropylene) and PE (polyethylene) and dries readily with a short dry step (80° C. for 1 hour).

iii. Selection of Support Material

PP and PE have been highlighted as the non-fluorinated support materials. There are several commercially available options with a variety of properties to evaluate and inform custom support design.

iv. Preparation of Support Material Prior to rAEM Fabrication

Washing PP and PE supports with an ethanol provides improved rAEM features.

v. Method of Incorporating Polymer into Support to Create rAEM

The following parameters for spray-coating were explored:
Manual versus automated 3D printing
Single-sided versus double-sided coats
Velocity of spray
Number of coats

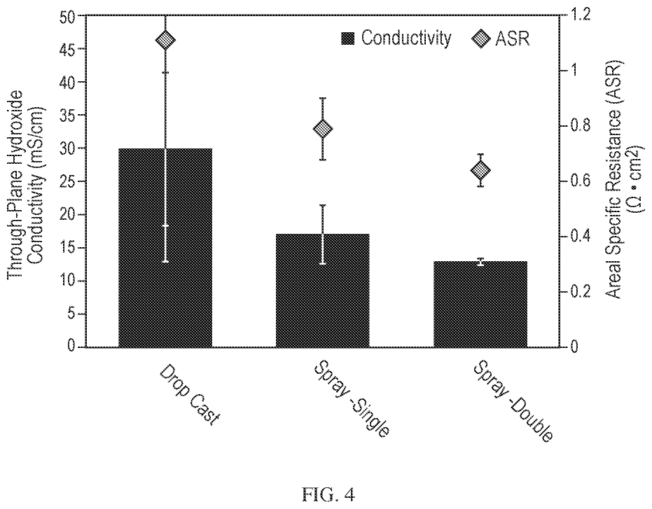

In addition to better uniformity and handling, the spray coating technique produced rAEMs with comparatively lower ASR (FIG. 4). The double coat method offered a small improvement and was the selected method for current development.

Tetrakis®-BXL AEMs offered unexpected processing advantages. Polymers containing benzophenone are significantly more solvent processable, even at small levels of incorporation. The Tetrakis®-BXL polymers are easily solution cast from mixtures of n-propanol and water. The boiling points of water (100° C.) and n-propanol (98° C.) are relatively low and similar to each other, which provides simplified and uniform drying. This is an improvement over more typical solution casting from dimethylformamide (DMF), which has a boiling point of 153° C. AEMs and rAEMs cast from DMF required a two-step drying system: 1) several hours at 80° C./ambient pressure followed by 2) overnight at 125° C./under vacuum.

3. Characterization of the Tetrakis®-BXL rAEMs

The following studies were performed in order to characterize the rAEMs:
Through-plane hydroxide conductivity & ASR of rAEMs were measured.
Values at ambient temperature were compared for support selection and fabrication optimization.
Conducted tensile testing to determine stress and strain at break for selected rAEMs.
Conducted water uptake & swelling studies of the rAEMs.
Conducted in-plane hydroxide conductivity of selected rAEMs.
Conducted IEC of selected rAEMs.

A. Characterization studies focused on the following variables: polymer load, polymer volume and thickness of the rAEM were performed.

The following materials were evaluated.

ia. PE (Teijin) Spray-Coated with T-28-120-BXL2 and UV Cross-Linked

In the range of support thicknesses evaluated (20-80 μm), lower ASR correlated to thinner rAEMs (dry and wet at RT). In the range of polymer loading evaluated (1.0-5.3 mg/cm₂), lower ASR generally trended with lower loading and polymer volume, with preferred values below 3 mg/cm². Low ASR tracked with low conductivity—ASR is prioritized over conductivity

iia. PE Spray-Coated with T-46-120-BXL2 and UV Cross-Linked

At RT, higher polymer volume led to lower ASR and higher conductivity in the range of polymer volume evaluated (582-743 mg/cm₃; 1.5 mg/cm₂ for each). At 80° C., the trend disappeared, and ASR was the same for all polymer volumes. Swelling in the Z-direction (thickness) appeared to the same at RT and 80° C. Thin rAEMs were produced with dry thicknesses below 30 μm. Conductivity and ASR appear to track inversely—lower ASR paired with higher conductivity. Gurley number and porosity of supports do not appear to impact rAEM properties.

iiia. PP (Celgard) Spray-Coated with T-28-120-BXL2 and UV Cross-Linked

rAEMs prepared with Celgard PP supports had uniform trends in properties—higher polymer loading resulted in higher polymer volume, which lead to higher dry and wet thicknesses. The trends may suggest advantages for fabrication with PP supports. Higher loading (0.2-4.0 mg/cm²) resulted in higher conductivity and lower ASR.

B. Additional studies were performed to evaluate the properties of PP (Celgard)-based rAEM as a function of the support Gurley values and polymer composition.

ib. PP (Celgard) Spray-Coated with T-28-120-BXL2 and UV Cross-Linked: Support Selection Based on Gurley Variation

Mesoporous PP supports with a range of Gurley values were evaluated and it was demonstrated that the lowest Gurley values produced both the lowest ASRs and highest conductivity. PP supports with Gurley values of 100 sec/dL were selected for additional fabrication and polymer composition evaluation. Generally higher porosity resulted in lower ASR and higher conductivity. Clear trends were not observed for support thickness, wet thickness, or polymer volume.

iib. Evaluation of Polymer Composition in PP (Celgard)

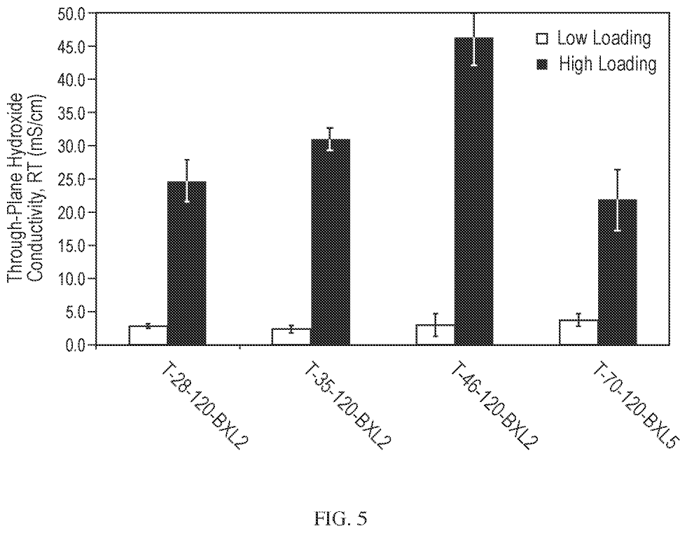

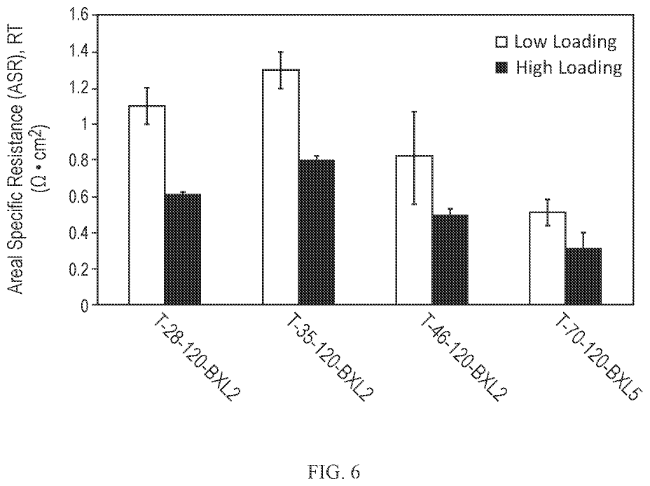

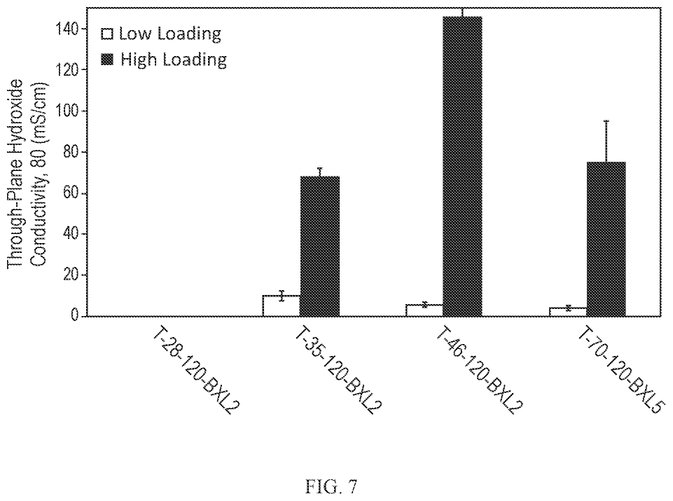

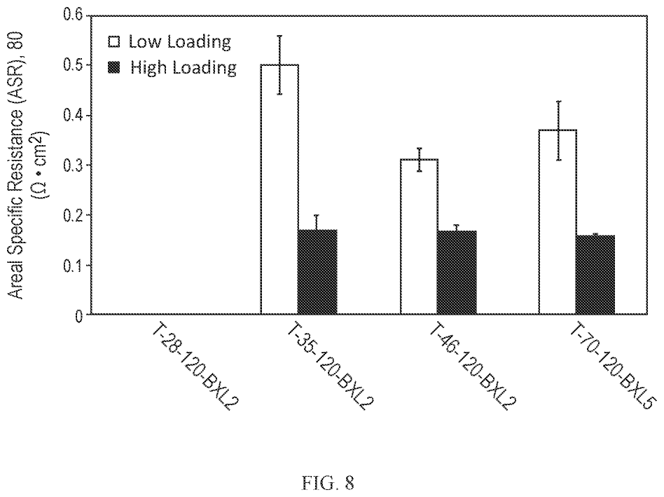

Effect of the cation content in the Tetrakis®-BXL polymers on the rAEM performance was explored (FIGS. 5-8). Across polymer compositions, higher loading resulted in higher conductivity and lower ASR. At RT, higher conductivity and lower ASR were observed with increases in cation content in the polymer; however, the trend is less clear at 80° C. Optimization of cation content and cross-linking density requires evaluation beyond conductivity and ASR, such as water uptake and mechanical properties. Higher cross-linking density in rAEMs with 70% cation content resulted in higher conductivity and lower ASR at 80° C. An increase in water uptake/swelling was also observed with increase in cross-link density, yet the values were similar between RT and 80° C.

C. PP-supported Tetrakis®-BXL rAEMs, unsupported cross-linked Tetrakis®-BXL AEM, and non-crosslinked Tetrakis® were characterized with respect to a number of important AEM parameters (Table 7).

TABLE 7
Characterization of PP-supported Tetrakis ®-BXL
rAEMs and unsupported Tetrakis ®-BXL
and Tetrakis ® AEMs.

| | T-23- | T-28- | PP-T-46- | PP-T-70- |
|---|---|---|---|---|
| Measurement | 300 | 120-BXL5 | 120-BXL2 | 120-BXL5 |
| Conductivity (IP, RT) | 28.7 | 25.6 | 19.8 | 11.6 |
| error | 4.3 | 1.2 | 1.6 | 3.39 |
| Conductivity (IP, 80) | 28.0 | 59.9 | 25.5 | 33.98 |
| error | 3.7 | 8.3 | 2.60 | 9.09 |
| Conductivity (TP, RT) | 26.4 | 27.6 | 24.10 | 16.87 |
| error | 1.5 | 2.1 | 6.30 | 2.49 |
| Conductivity (TP, 80) | 80.7 | 72.9 | 214.00 | 27.06 |
| error | 8.4 | 7.3 | 24.00 | 4.36 |
| ASR (RT) | 0.21 | 0.19 | 0.46 | 0.27 |
| error | 0.02 | 0.01 | 0.03 | 0.03 |
| ASR (80) | 0.10 | 0.05 | 0.12 | 0.16 |
| error | 0.01 | 0.01 | 0.02 | 0.02 |
| IEC | 1.04 | 0.90 | 1.32 | 4.12 |
| error | 0.06 | 0.10 | 0.57 | 0.74 |
| Water Uptake (Mass, RT) | 99.0 | 56.6 | 227.00 | 129.9 |
| error | 21.9 | 9.7 | 24.97 | 29.65 |
| Water Uptake (Mass, 80) | 317 | 96.9 | 660.00 | 113.8 |
| error | 78 | 13.3 | 158.00 | 3.45 |
| Swelling (X-Y, RT) | 21.4 | 14.5 | 1.00 | 1 |
| error | 1.1 | 5.0 | 3.87 | |
| Swelling (X-Y, 80) | 48.7 | 16.9 | 1.00 | 1 |
| error | 2.8 | 5.2 | 5.32 | |
| Swelling (Z, RT) | 11.6 | 1 | 203.50 | 48.08 |
| error | 3.7 | | 28.56 | 16.61 |
| Swelling (Z, 80) | 60.2 | 1 | 384.90 | 39.44 |
| error | 23 | | 143.30 | 20.46 |
| Stress @ Break | 6.3 | 20.3 | 9.37 | 52.3 |
| error | 0.8 | 1.4 | 0.44 | 0.2 |
| Strain @ Break | 1160 | 165 | 460.00 | 77.8 |
| error | 355 | 11 | 34.00 | 3.1 |
| HSF | 23.8 | 9.8 | 460.0 | 77.8 |
| Hydration Number | 53 | 35 | 96 | 18 |
| Hydroxide Mobility | 27 | 67 | 19 | 8 |

D. PP-supported Tetrakis®-BXL rAEMs were also compared to commercially available AEMs (Table 8).

TABLE 8
Characterization of PP-supported Tetrakis ®-BXL rAEMs and commercial AEMs.

| | PP-T-46- | PP-T-70- | Pention | Versogen | Xion Durion |
|---|---|---|---|---|---|
| Measurement | 120-BXL2 | 120-BXL5 | 72-30-15 | PiperION-15-rAEM | LMW-215-30 |
| Conductivity (IP, RT) | 19.8 | 11.6 | 29.8 | 35.4 | 39.2 |
| error | 1.6 | 3.39 | 2.8 | 5.6 | 4.3 |
| Conductivity (IP, 80) | 25.5 | 33.98 | 108.7 | 52.9 | 78.6 |
| error | 2.60 | 9.09 | 14.0 | 1.0 | 23.3 |
| Conductivity (TP, RT) | 24.10 | 16.87 | 23.4 | 16.1 | 31.4 |
| error | 6.30 | 2.49 | 2.8 | 2.2 | 5.6 |
| Conductivity (TP, 80) | 214.00 | 27.06 | 40.3 | | |
| error | 24.00 | 4.36 | 5.7 | | |
| ASR (RT) | 0.46 | 0.27 | 0.17 | 0.19 | 0.24 |
| error | 0.03 | 0.03 | 0.02 | 0.02 | 0.04 |
| ASR (80) | 0.12 | 0.16 | 0.11 | | |
| error | 0.02 | 0.02 | 0.02 | | |
| IEC | 1.32 | 4.12 | 2.40 | 2.90 | 1.54 |
| error | 0.57 | 0.74 | 0.30 | 0.70 | 0.06 |
| Water Uptake (Mass, RT) | 227.00 | 129.9 | 65.3 | 66.1 | 133 |
| error | 24.97 | 29.65 | 19.9 | 17.6 | 31 |
| Water Uptake (Mass, 80) | 660.00 | 113.8 | 56.1 | 109.5 | 500 |
| error | 158.00 | 3.45 | 4.9 | 10.8 | 47 |
| Swelling (X-Y, RT) | 1.00 | 1 | 8.5 | 4.54 | 1.4 |
| error | 3.87 | | 12.2 | 4.4 | 1.3 |
| Swelling (X-Y, 80) | 1.00 | 1 | 8.8 | | 95.8 |
| error | 5.32 | | 15.9 | | 8.5 |
| Swelling (Z, RT) | 203.50 | 48.08 | 9.3 | 51.6 | 126 |
| error | 28.56 | 16.61 | 3.6 | 16.9 | 30 |
| Swelling (Z, 80) | 384.90 | 39.44 | 18.8 | 69.8 | 448 |
| error | 143.30 | 20.46 | 3 | 11.7 | 23 |
| Stress @ Break | 9.37 | 52.3 | 27.7 | | 38.2 |
| error | 0.44 | 0.2 | 3.4 | | 2.6 |
| Strain @ Break | 460.00 | 77.8 | 5.1 | | 6.6 |
| error | 34.00 | 3.1 | 1.6 | | 1.1 |
| HSF | 460.0 | 77.8 | 0.6 | | 0.1 |
| Hydration Number | 96 | 18 | 15 | 13 | 48 |
| Hydroxide Mobility | 19 | 8 | 45 | 18 | 51 |

Structural characteristics of the Xion Durion are listed in Table 1. Versogen rAEM is an AEM reinforced with microporous ePTFE support, comprising AIE of the following structure:

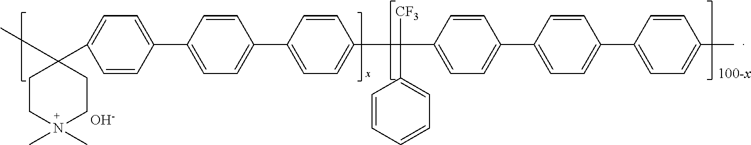

### Definitions

Definitions of specific functional groups and chemical terms are described in more detail below. The chemical elements are identified in accordance with the Periodic Table of the Elements, CAS version, Handbook of Chemistry and Physics, 75^{th} Ed., inside cover, and specific functional groups are generally defined as described therein. Additionally, general principles of organic chemistry, as well as specific functional moieties and reactivity, are described in Organic Chemistry, Thomas Sorrell, University Science Books, Sausalito, 1999; Smith and March, March's Advanced Organic Chemistry, 5^{th} Edition, John Wiley & Sons, Inc., New York, 2001; Larock, Comprehensive Organic Transformations, VCH Publishers, Inc., New York, 1989; and Carruthers, Some Modern Methods of Organic Synthesis, 3^{rd} Edition, Cambridge University Press, Cambridge, 1987.

Compounds described herein can comprise one or more asymmetric centers, and thus can exist in various stereoisomeric forms, e.g., enantiomers and/or diastereomers. For example, the compounds described herein can be in the form of an individual enantiomer, diastereomer or geometric isomer, or can be in the form of a mixture of stereoisomers, including racemic mixtures and mixtures enriched in one or more stereoisomer. Isomers can be isolated from mixtures by methods known to those skilled in the art, including chiral high pressure liquid chromatography (HPLC) and the formation and crystallization of chiral salts; or preferred isomers can be prepared by asymmetric syntheses. See, for example, Jacques et al., Enantiomers, Racemates and Resolutions, Wiley Interscience, New York, 1981; Wilen et al., Tetrahedron 33:2725 (1977); Eliel, E. L. Stereochemistry of Carbon Compounds, McGraw-Hill, N Y, 1962; and Wilen, S. H., Tables of Resolving Agents and Optical Resolutions p. 268, E. L. Eliel, Ed., Univ. of Notre Dame Press, Notre Dame, IN 1972. The invention additionally encompasses compounds as individual isomers substantially free of other isomers, and alternatively, as mixtures of various isomers.

In a formula, [symbol] or [symbol] is a single or double bond.

When a range of values is listed, it is intended to encompass each value and sub-range
within the range. For example, “C₁₋₆ alkyl” is intended to encompass C₁, C₂, C₃, C₄, C₅, C₆, C₁₋₆, C₁₋₅, C₁₋₄, C₁₋₃, C₁₋₂, C₂₋₆, C₂₋₅, C₂₋₄, C₂₋₃, C₃₋₆, C₃₋₅, C₃₋₄, C₄₋₆, C₄₋₅, and C₅₋₆ alkyl.

The term “alkyl” refers to a radical of a straight-chain or branched saturated hydrocarbon group having from 1 to 18 carbon atoms (“C₁₋₁₈ alkyl”). In some embodiments, an alkyl group has 1 to 12 carbon atoms (“C₁₋₁₂ alkyl”). In some embodiments, an alkyl group has 1 to 8 carbon atoms (“C₁₋₈ alkyl”). In some embodiments, an alkyl group has 1 to 6 carbon atoms (“C₁₋₆ alkyl”). In some embodiments, an alkyl group has 1 to 3 carbon atoms (“C₁₋₃ alkyl”). In some embodiments, an alkyl group has 2 to 6 carbon atoms (“C₂₋₆ alkyl”). Examples of C₁₋₆ alkyl groups include methyl (C₁), ethyl (C₂), propyl (C₃) (e.g., n-propyl, isopropyl), butyl (C₄) (e.g., n-butyl, tert-butyl, sec-butyl, iso-butyl), pentyl (C₅) (e.g., n-pentyl, 3-pentanyl, amyl, neopentyl, 3-methyl-2-butanyl, tertiary amyl), and hexyl (C₆) (e.g., n-hexyl). Additional examples of alkyl groups include n-heptyl (C₇), n-octyl (C₈), and the like. Unless otherwise specified, each instance of an alkyl group is independently unsubstituted (an “unsubstituted alkyl”) or substituted (a “substituted alkyl”). In certain embodiments, the alkyl group is an unsubstituted C₁₋₁₂ alkyl (such as unsubstituted C₁₋₆ alkyl, e.g., —CH₃ (Me), unsubstituted ethyl (Et), unsubstituted propyl (Pr, e.g., unsubstituted n-propyl (n-Pr), unsubstituted isopropyl (i-Pr)), unsubstituted butyl (Bu, e.g., unsubstituted n-butyl (n-Bu), unsubstituted tert-butyl (tert-Bu or t-Bu), unsubstituted sec-butyl (sec-Bu), unsubstituted isobutyl (i-Bu)). In certain embodiments, the alkyl group is a substituted C₁₋₁₂ alkyl (such as substituted C₁₋₆ alkyl, e.g., —CF₃, Bn).

The term “haloalkyl” refers to a substituted alkyl group, wherein one or more of the hydrogen atoms are independently replaced by a halogen, e.g., fluoro, bromo, chloro, or iodo. In some embodiments, the haloalkyl moiety has 1 to 12 carbon atoms (“C₁₋₁₂ haloalkyl”). In some embodiments, the haloalkyl moiety has 1 to 6 carbon atoms (“C₁₋₆ haloalkyl”). In some embodiments, the haloalkyl moiety has 1 to 4 carbon atoms (“C₁₋₄ haloalkyl”). In some embodiments, the haloalkyl moiety has 1 to 3 carbon atoms (“C₁₋₃ haloalkyl”). In some embodiments, the haloalkyl moiety has 1 to 2 carbon atoms (“C₁₋₂ haloalkyl”). Examples of haloalkyl groups include —CHF₂, —CH₂F, —CF₃, —CH₂CF₃, —CF₂CF₃, —CF₂CF₂CF₃, —CCl₃, —CFCl₂, —CF₂Cl, and the like.

The term “alkoxy” refers to an alkyl group, as defined herein, appended to the parent molecular moiety through an oxygen atom. In some embodiments, the alkoxy moiety has 1 to 12 carbon atoms (“C₁₋₁₂ alkoxy”). In some embodiments, the alkoxy moiety has 1 to 6 carbon atoms (“C₁₋₆ alkoxy”). In some embodiments, the alkoxy moiety has 1 to 4 carbon atoms (“C₁₋₄ alkoxy”). In some embodiments, the alkoxy moiety has 1 to 3 carbon atoms (“C₁₋₃ alkoxy”). In some embodiments, the alkoxy moiety has 1 to 2 carbon atoms (“C₁₋₂ alkoxy”). Representative examples of alkoxy include, but are not limited to, methoxy, ethoxy, propoxy, 2-propoxy, butoxy and tert-butoxy.

In some embodiments, “cycloalkyl” is a radical of a saturated hydrocarbon monocyclic or polycyclic group having from 3 to 18 ring carbon atoms (“C₃₋₁₈ cycloalkyl”). In some embodiments, a cycloalkyl group has 3 to 12 ring carbon atoms (“C₃₋₁₂ cycloalkyl”). In some embodiments, a cycloalkyl group has 3 to 8 ring carbon atoms (“C₃₋₈cycloalkyl”). In some embodiments, a cycloalkyl group has 5 to 12 ring carbon atoms (“5-12 cycloalkyl”). In some embodiments, a cycloalkyl group has 4 to 6 ring carbon atoms (“C₄₋₆ cycloalkyl”). In some embodiments, a cycloalkyl group has 5 to 6 ring carbon atoms (“C₅₋₆ cycloalkyl”). In some embodiments, a cycloalkyl group has 5 to 7 ring carbon atoms (“C₅₋₇ cycloalkyl”). A polycyclic cycloalkyl group can be, for example, bycyclic, tricyclic, or tetracyclic. A polycyclic cycloalkyl group can contain fused cycloalkyl rings. A polycyclic cycloalkyl group can be a spirocyclic cycloalkyl group or a bridged cycloalkyl group. Examples of C₅₋₆ cycloalkyl groups include cyclopentyl (C₅) and cyclohexyl (C₆). Examples of C₃₋₆ cycloalkyl groups include the aforementioned C₅₋₆ cycloalkyl groups as well as cyclopropyl (C₃) and cyclobutyl (C₄). Examples of C₃₋₈ cycloalkyl groups include the aforementioned C₃₋₆ cycloalkyl groups as well as cycloheptyl (C₇) and cyclooctyl (C₈). Unless otherwise specified, each instance of a cycloalkyl group is independently unsubstituted (an “unsubstituted cycloalkyl”) or substituted (a “substituted cycloalkyl”) with one or more substituents. In certain embodiments, the cycloalkyl group is an unsubstituted C₃₋₁₂ cycloalkyl. In certain embodiments, the cycloalkyl group is a substituted C₃₋₁₂ cycloalkyl. In certain embodiments, the cycloalkyl group is an unsubstituted C₅₋₁₂ cycloalkyl. In certain embodiments, the cycloalkyl group is a substituted C₅₋₁₂ cycloalkyl.

In some embodiments, “cycloalkenyl” is a non-aromatic radical of a hydrocarbon monocyclic or polycyclic group having at least one double bond and from 4 to 18 ring carbon atoms (“C₄₋₁₈cycloalkenyl”). In some embodiments, a cycloalkenyl group has 4 to 12 ring carbon atoms (“C₄₋₁₂ cycloalkenyl”). In some embodiments, a cycloalkyl group has 4 to 8 ring carbon atoms (“C₄₋₈cycloalkenyl”). In some embodiments, a cycloalkenyl group has 5 to 12 ring carbon atoms (“5-12 cycloalkenyl”). In some embodiments, a cycloalkenyl group has 7 to 8 ring carbon atoms (“C₇₋₈cycloalkenyl”). A polycyclic cycloalkenyl group can be, for example, bycyclic, tricyclic, or tetracyclic. A polycyclic cycloalkenyl group can contain a cycloalkenyl ring fused to another cycloalkenyl ring, a cycloalkyl ring, or a heterocyclyl ring. A polycyclic cycloalkenyl group can be a spirocyclic cycloalkenyl group or a bridged cycloalkenyl group. Exemplary cycloalkenyl groups include, without limitation, cyclooctenyl, bicyclooctenyl, and norbornenyl.

The term “aryl” refers to a radical of a monocyclic or polycyclic (e.g., bicyclic or tricyclic) 4n+2 aromatic ring system (e.g., having 6, 10, or 14 π electrons shared in a cyclic array) having 6-14 ring carbon atoms and zero heteroatoms provided in the aromatic ring system (“C₆₋₁₄ aryl”). In some embodiments, an aryl group has 6 ring carbon atoms (“C₆ aryl”; e.g., phenyl). In some embodiments, an aryl group has 10 ring carbon atoms (“C₁₀ aryl”; e.g., naphthyl such as 1-naphthyl and 2-naphthyl). In some embodiments, an aryl group has 14 ring carbon atoms (“C₁₄ aryl”; e.g., anthracyl). “Aryl” also includes ring systems wherein the aryl ring, as defined above, is fused with one or more carbocyclyl or heterocyclyl groups wherein the radical or point of attachment is on the aryl ring, and in such instances, the number of carbon atoms continue to designate the number of carbon atoms in the aryl ring system. Unless otherwise specified, each instance of an aryl group is independently unsubstituted (an “unsubstituted aryl”) or substituted (a “substituted aryl”) with one or more substituents. In certain embodiments, the aryl group is an unsubstituted C₆₋₁₂ aryl. In certain embodiments, the aryl group is a substituted C₆₋₁₂ aryl.

The term “aryloxy” refers to an aryl group, as defined herein, appended to the parent molecular moiety through an oxygen atom. In some embodiments, the aryloxy moiety has 6 to 12 carbon atoms (“C₆₋₁₂ aryloxy”). In some embodiments, the aryloxy moiety has 6 to 10 carbon atoms (“C₆₋₁₀ aryloxy”). Representative examples of aryloxy include, but are not limited to, phenoxy and naphthoxy.

The term “heterocyclyl” or “heterocyclic” refers to a radical of a 3- to 16-membered saturated, unsaturated non-aromatic, or aromatic ring system having ring carbon atoms and 1 to 4 ring heteroatoms, wherein each heteroatom is independently selected from nitrogen, oxygen, and sulfur (“5-16 membered heterocyclyl”). In heterocyclyl groups that contain one or more nitrogen atoms, the point of attachment can be a carbon or nitrogen atom, as valency permits. A heterocyclyl group can either be monocyclic (“monocyclic heterocyclyl”) or polycyclic (e.g., a fused, bridged or spiro ring system such as a bicyclic system (“bicyclic heterocyclyl”) or tricyclic system (“tricyclic heterocyclyl”). Heterocyclyl polycyclic ring systems can include one or more heteroatoms in one or both rings. “Heterocyclyl” also includes ring systems wherein the heterocyclyl ring, as defined above, is fused with one or more cycloalkyl groups wherein the point of attachment is either on the cycloalkyl or heterocyclyl ring, or ring systems wherein the heterocyclyl ring, as defined above, is fused with one or more aryl groups, wherein the point of attachment is on the heterocyclyl ring, and in such instances, the number of ring members continue to designate the number of ring members in the combined fused ring system. Unless otherwise specified, each instance of heterocyclyl is independently unsubstituted (an “unsubstituted heterocyclyl”) or substituted (a “substituted heterocyclyl”) with one or more substituents. In certain embodiments, the heterocyclyl group is an unsubstituted 5-12 membered heterocyclyl. In certain embodiments, the heterocyclyl group is a substituted 5-12 membered heterocyclyl.

In some embodiments, a heterocyclyl group is a 5-10 membered non-aromatic ring system having ring carbon atoms and 1-4 ring heteroatoms, wherein each heteroatom is independently selected from nitrogen, oxygen, and sulfur (“5-10 membered heterocyclyl”). In some embodiments, a heterocyclyl group is a 5-8 membered non-aromatic ring system having ring carbon atoms and 1-4 ring heteroatoms, wherein each heteroatom is independently selected from nitrogen, oxygen, and sulfur (“5-8 membered heterocyclyl”). In some embodiments, a heterocyclyl group is a 5-6 membered non-aromatic ring system having ring carbon atoms and 1-4 ring heteroatoms, wherein each heteroatom is independently selected from nitrogen, oxygen, and sulfur (“5-6 membered heterocyclyl”). In some embodiments, the 5-6 membered heterocyclyl has 1-3 ring heteroatoms selected from nitrogen, oxygen, and sulfur. In some embodiments, the 5-6 membered heterocyclyl has 1-2 ring heteroatoms selected from nitrogen, oxygen, and sulfur. In some embodiments, the 5-6 membered heterocyclyl has 1 ring heteroatom selected from nitrogen, oxygen, and sulfur.

As used herein, the term “heterocycloalkenyl” refers to an unsaturated non-aromatic heterocyclyl group as described above, comprising one or more double bonds. In some embodiments, heterocycloalkenyl groups are bicyclic bridge moieties. In some embodiments, heterocycloalkenyl groups are bicyclic fused moieties. Exemplary heterocycloalkenyl groups include, without limitation, 7-oxabicyclo[2.2.1]hept-2-ene, 7-azabicyclo[2.2.1]hept-2-ene, and 7-methyl-7-azabicyclo[2.2.1]hept-2-ene.

Exemplary 3-membered heterocyclyl groups containing 1 heteroatom include, without limitation, aziridinyl, oxiranyl, and thiiranyl. Exemplary 4-membered heterocyclyl groups containing 1 heteroatom include, without limitation, azetidinyl, oxetanyl, and thietanyl. Exemplary 5-membered heterocyclyl groups containing 1 heteroatom include, without limitation, tetrahydrofuranyl, dihydrofuranyl, tetrahydrothiophenyl, dihydrothiophenyl, pyrrolidinyl, dihydropyrrolyl, and pyrrolyl-2,5-dione. Exemplary 5-membered non-aromatic heterocyclyl groups containing 1 heteroatom include, without limitation, tetrahydrofuranyl, dihydrofuranyl, tetrahydrothiophenyl, dihydrothiophenyl, pyrrolidinyl, dihydropyrrolyl, and pyrrolyl-2,5-dione. Exemplary 5-membered heterocyclyl groups containing 2 heteroatoms include, without limitation, dioxolanyl, oxathiolanyl and dithiolanyl. Exemplary 5-membered heterocyclyl groups containing 3 heteroatoms include, without limitation, triazolinyl, oxadiazolinyl, and thiadiazolinyl. Exemplary 6-membered heterocyclyl groups containing 1 heteroatom include, without limitation, piperidinyl, tetrahydropyranyl, dihydropyridinyl, and thianyl. Exemplary 6-membered heterocyclyl groups containing 2 heteroatoms include, without limitation, piperazinyl, morpholinyl, dithianyl, and dioxanyl. Exemplary 6-membered heterocyclyl groups containing 3 heteroatoms include, without limitation, triazinyl. Exemplary 7-membered heterocyclyl groups containing 1 heteroatom include, without limitation, azepanyl, oxepanyl and thiepanyl. Exemplary 8-membered heterocyclyl groups containing 1 heteroatom include, without limitation, azocanyl, oxecanyl and thiocanyl. Exemplary bicyclic heterocyclyl groups include, without limitation, indolinyl, isoindolinyl, dihydrobenzofuranyl, dihydrobenzothienyl, tetrahydrobenzothienyl, tetrahydrobenzofuranyl, tetrahydroindolyl, tetrahydroquinolinyl, tetrahydroisoquinolinyl, decahydroquinolinyl, decahydroisoquinolinyl, octahydrochromenyl, octahydroisochromenyl, decahydronaphthyridinyl, decahydro-1,8-naphthyridinyl, octahydropyrrolo[3,2-b]pyrrole, indolinyl, phthalimidyl, naphthalimidyl, chromanyl, chromenyl, 1H-benzo[e][1,4]diazepinyl, 1,4,5,7-tetrahydropyrano[3,4-b]pyrrolyl, 5,6-dihydro-4H-furo[3,2-b]pyrrolyl, 6,7-dihydro-5H-furo[3,2-b]pyranyl, 5,7-dihydro-4H-thieno[2,3-c]pyranyl, 2,3-dihydro-1H-pyrrolo[2,3-b]pyridinyl, 2,3-dihydrofuro[2,3-b]pyridinyl, 4,5,6,7-tetrahydro-1H-pyrrolo[2,3-b]pyridinyl, 4,5,6,7-tetrahydrofuro[3,2-c]pyridinyl, 4,5,6,7-tetrahydrothieno[3,2-b]pyridinyl, 1,2,3,4-tetrahydro-1,6-naphthyridinyl, and the like.

In some embodiments, the term “heterocyclyl” refers to a radical of a 5-16 membered monocyclic or polycyclic (e.g., bicyclic, tricyclic) 4n+2 aromatic ring system (e.g., having 6, 10, or 14 π electrons shared in a cyclic array), also referred to as “heteroaryl”, having ring carbon atoms and 1-4 ring heteroatoms provided in the aromatic ring system, wherein each heteroatom is independently selected from nitrogen, oxygen, sulfur. In heteroaryl groups that contain one or more nitrogen atoms, the point of attachment can be a carbon or nitrogen atom, as valency permits. Heteroaryl polycyclic ring systems can include one or more heteroatoms in one or both rings. “Heteroaryl” includes ring systems wherein the heteroaryl ring, as defined above, is fused with one or more carbocyclyl or heterocyclyl groups wherein the point of attachment is on the heteroaryl ring, and in such instances, the number of ring members continue to designate the number of ring members in the heteroaryl ring system. “Heteroaryl” also includes ring systems wherein the heteroaryl ring, as defined above, is fused with one or more aryl groups wherein the point of attachment is either on the aryl or heteroaryl ring, and in such instances, the number of ring members designates the number of ring members in the fused polycyclic (aryl/heteroaryl) ring system. Polycyclic heteroaryl groups wherein one ring does not contain a heteroatom (e.g., indolyl, quinolinyl, carbazolyl, and the like) the point of attachment can be on either ring, i.e., either the ring bearing a heteroatom (e.g., 2-indolyl) or the ring that does not contain a heteroatom (e.g., 5-indolyl).

In some embodiments, a heteroaryl group is a 5-12 membered aromatic ring system having ring carbon atoms and 1-4 ring heteroatoms provided in the aromatic ring system, wherein each heteroatom is independently selected from nitrogen, oxygen, and sulfur (“5-12 membered heteroaryl”). In some embodiments, a heteroaryl group is a 5-8 membered aromatic ring system having ring carbon atoms and 1-4 ring heteroatoms provided in the aromatic ring system, wherein each heteroatom is independently selected from nitrogen, oxygen, and sulfur (“5-8 membered heteroaryl”). In some embodiments, a heteroaryl group is a 5-6 membered aromatic ring system having ring carbon atoms and 1-4 ring heteroatoms provided in the aromatic ring system, wherein each heteroatom is independently selected from nitrogen, oxygen, and sulfur (“5-6 membered heteroaryl”). In some embodiments, the 5-6 membered heteroaryl has 1-3 ring heteroatoms selected from nitrogen, oxygen, and sulfur. In some embodiments, the 5-6 membered heteroaryl has 1-2 ring heteroatoms selected from nitrogen, oxygen, and sulfur. In some embodiments, the 5-6 membered heteroaryl has 1 ring heteroatom selected from nitrogen, oxygen, and sulfur. Unless otherwise specified, each instance of a heteroaryl group is independently unsubstituted (an “unsubstituted heteroaryl”) or substituted (a “substituted heteroaryl”) with one or more substituents. In certain embodiments, the heteroaryl group is an unsubstituted 5-14 membered heteroaryl. In certain embodiments, the heteroaryl group is a substituted 5-14 membered heteroaryl.

Exemplary 5-membered heteroaryl groups containing 1 heteroatom include, without limitation, pyrrolyl, furanyl, and thiophenyl. Exemplary 5-membered heteroaryl groups containing 2 heteroatoms include, without limitation, imidazolyl, pyrazolyl, oxazolyl, isoxazolyl, thiazolyl, and isothiazolyl. Exemplary 5-membered heteroaryl groups containing 3 heteroatoms include, without limitation, triazolyl, oxadiazolyl, and thiadiazolyl. Exemplary 5-membered heteroaryl groups containing 4 heteroatoms include, without limitation, tetrazolyl. Exemplary 6-membered heteroaryl groups containing 1 heteroatom include, without limitation, pyridinyl. Exemplary 6-membered heteroaryl groups containing 2 heteroatoms include, without limitation, pyridazinyl, pyrimidinyl, and pyrazinyl. Exemplary 6-membered heteroaryl groups containing 3 or 4 heteroatoms include, without limitation, triazinyl and tetrazinyl, respectively. Exemplary 7-membered heteroaryl groups containing 1 heteroatom include, without limitation, azepinyl, oxepinyl, and thiepinyl. Exemplary 5,6-bicyclic heteroaryl groups include, without limitation, indolyl, isoindolyl, indazolyl, benzotriazolyl, benzothiophenyl, isobenzothiophenyl, benzofuranyl, benzoisofuranyl, benzimidazolyl, benzoxazolyl, benzisoxazolyl, benzoxadiazolyl, benzthiazolyl, benzisothiazolyl, benzthiadiazolyl, indolizinyl, and purinyl. Exemplary 6,6-bicyclic heteroaryl groups include, without limitation, naphthyridinyl, pteridinyl, quinolinyl, isoquinolinyl, cinnolinyl, quinoxalinyl, phthalazinyl, and quinazolinyl. Exemplary tricyclic heteroaryl groups include, without limitation, phenanthridinyl, dibenzofuranyl, carbazolyl, acridinyl, phenothiazinyl, phenoxazinyl, and phenazinyl.

The term “unsaturated” or “partially unsaturated” refers to a moiety that includes at least one double or triple bond.

The term “saturated” refers to a moiety that does not contain a double or triple bond, i.e., the moiety only contains single bonds.

Affixing the suffix “-ene” to a group indicates the group is a divalent moiety, e.g., alkylene is the divalent moiety of alkyl, arylene is the divalent moiety of aryl, cycloalkylene is a divalent moiety of cycloalkyl, and heterocyclylene is the divalent moiety of hereocyclyl.

The term “C_{x-y}” when used in conjunction with a chemical moiety, such as, acyl, acyloxy, alkyl, alkenyl, alkynyl, or alkoxy is meant to include groups that contain from x to y carbons in the chain. For example, the term “C_{x-y}alkyl” refers to substituted or unsubstituted saturated hydrocarbon groups, including straight-chain alkyl and branched-chain alkyl groups that contain from x to y carbons in the chain, including haloalkyl groups such as trifluoromethyl and 2,2,2-trifluoroethyl, etc. C₀ alkyl indicates a hydrogen where the group is in a terminal position, a bond if internal.

A group is optionally substituted unless expressly provided otherwise. The term “optionally substituted” refers to being substituted or unsubstituted. In certain embodiments, alkyl, cycloalkyl, cycloalkenyl, heterocyclyl, heterocycloalkenyl, aryl, and heteroaryl groups and the corresponding divalent moieties are optionally substituted. “Optionally substituted” refers to a group which may be substituted or unsubstituted (e.g., “substituted” or “unsubstituted” alkyl, “substituted” or “unsubstituted” cycloalkyl, “substituted” or “unsubstituted” cycloalkenyl, “substituted” or “unsubstituted” heterocyclyl, “substituted” or “unsubstituted” heterocycloalkenyl, “substituted” or “unsubstituted” aryl or “substituted” or “unsubstituted” heteroaryl group). In general, the term “substituted” means that at least one hydrogen present on a group is replaced with a permissible substituent, e.g., a substituent which upon substitution results in a stable compound, e.g., a compound which does not spontaneously undergo transformation such as by rearrangement, cyclization, elimination, or other reaction. Unless otherwise indicated, a “substituted” group has a substituent at one or more substitutable positions of the group, and when more than one position in any given structure is substituted, the substituent is either the same or different at each position. The term “substituted” is contemplated to include substitution with all permissible substituents of organic compounds, and includes any of the substituents described herein that results in the formation of a stable compound. The present invention contemplates any and all such combinations in order to arrive at a stable compound. For purposes of this invention, heteroatoms such as nitrogen may have hydrogen substituents and/or any suitable substituent as described herein which satisfy the valencies of the heteroatoms and results in the formation of a stable moiety. The invention is not intended to be limited in any manner by the exemplary substituents described herein.

Exemplary carbon atom substituents include, but are not limited to, halogen, —CN, —NO₂, —N₃, —OH, F, Cl, Br, I, oxo, —SO₂H, —SO₃H, —OR^{aa}, —NH(R′)₂, —N(R^{aa})₂, —N(R^{aa})₃⁺X⁻, —SH, —SR^{aa}, —C(═O)R^{aa}, —CO₂H, —CHO, —CO₂R^{aa}, —OC(═O)R^{aa}, —OCO₂R^{aa}, —C(═O)N(R^{aa})₂, —OC(═O)N(R^{aa})₂, —NR^{aa}C(═O)R^{aa}, —NR^{aa}CO₂R^{aa}, —NR^{aa}C(═O)N(R^{aa})₂, —C(═NR^{aa})R^{aa}, —C(═O)NR^{aa}SO₂R^{aa}, —NR^{aa}SO₂R^{aa}, —SO₂N(R^{aa})₂, —SO₂R^{aa}, —SO₂OR^{aa}, —OSO₂R^{aa}, —S(═O)R^{aa}, —OS(═O)R^{aa}, —Si(R^{aa})₃, —OSi(R^{aa})₃, C₁₋₁₂ alkyl, C₁₋₁₂ haloalkyl, 3-16 membered heterocyclyl, and C₆₋₁₂ aryl, wherein X⁻ is a counterion and each instance of R^{aa} is, independently, selected from H, —OH, C₁₋₁₀ alkyl, C₁₋₁₀ haloalkyl, C₃₋₁₂ cycloalkyl, 5-16 membered heterocyclyl, and C₆₋₁₂ aryl, or two R^{aa} groups are joined to form a 3-16 membered heterocyclyl.

Numeric ranges are inclusive of the numbers defining the range. Measured and measureable values are understood to be approximate, taking into account significant digits and the error associated with the measurement. As used in this application, the terms “about” and “approximately” have their art-understood meanings; use of one vs the other does not necessarily imply different scope. Unless otherwise indicated, numerals used in this application, with or without a modifying term such as “about” or “approximately”, should be understood to encompass normal divergence and/or fluctuations as would be appreciated by one of ordinary skill in the relevant art. In certain embodiments, the term “approximately” or “about” refers to a range of values that fall within 10%, 9%, 8%, 7%, 6%, 5%, 4%, 3%, 2%, 1%, or less in either direction (greater than or less than) of a stated reference value unless otherwise stated or otherwise evident from the context (except where such number would exceed 100% of a possible value).

As used herein, the term “composite material” refers to a material made from two or more constituent materials with significantly different physical or chemical properties separated by a distinct interface. When combined, the two or more constituent materials produce a composite material with characteristics different from the individual components. The individual components remain separate and distinct within the composite material, thus differentiating composite materials from mixtures and solid solutions.

As used herein, the term “reinforcement” refers to any material that can provide mechanical support to the polymer without interfering with the function of the polymer. For example, a reinforcement can be mixed with the polymer, it can be impregnated with the polymer, or it can be coated with the polymer to provide the composite material. A reinforcement can be an inorganic material, such as a ceramic material, a polymer, or a composite of an inorganic material and a polymer, such as fiberglass.

As used herein, “support material” refers to a material having mechanical strength and chemical durability, which can be impregnated and/or coated with the polymer to provide the composite material. The support material can be made, for example, of a ceramic material or a polymer, such as a polyolefin, a polysulfone, or a polyamide. In some embodiments, the support comprises a polyimide, a polybenzimidazole, a polyphenylsulfone, a polyphenyl ether, cellulose nitrate, cellulose diacetate, cellulose triacetate, polypropylene, polyethylene, polyvinylidene fluoride, poly(phenylene sulfide), poly(vinyl chloride), polystyrene, poly(methyl methacrylate), polyacrylonitrile, polytetrafluoroethylene, polyetheretherketone, polycarbonate, polyvinyltrimethylsilane, polytrimethylsilylpropyne, poly(ether imide), poly(ether sulfone), polyoxadiazole, or poly(phenylene oxide), or a combination or copolymer thereof. The support material can be in a form of a film.

As used herein, the term “porous material impregnated with polymer” refers to a porous material that contains a polymer within its pores. A porous material can be impregnated with a polymer, for example, by soaking the material in a solution of the polymer or by spraying a solution of the polymer on the porous material. Alternatively, the porous material can be impregnated with a solution of one or more monomers, followed by a polymerization reaction within the pores of the material. Additionally, once a porous material impregnated with a polymer, the polymer can undergo further chemical transformations, such as cross-linking, within the pores of the material.

As used herein, the term “repeat unit” (also known as a monomer unit) refers to a chemical moiety which periodically repeats itself to produce the complete polymer chain (except for the end-groups) by linking the repeat units together successively. A polymer can contain one or more different repeat units.

As used herein, the “main chain” of a polymer, or the “backbone” of the polymer, is the series of bonded atoms that together create the continuous chain of the molecule. As used herein, a “side chain” of a polymer is the series of bonded atoms which are pendent from the main chain of a polymer.

As used herein, the term “cross-linked polymer” refers to a polymer in which two or more non-adjacent repeat units of the same main chain are connected via a cross-linking moiety. The term “cross-linking polymer” also refers to two or more different main chains connected via a plurality of cross-linking moieties.

As used herein, the term “cross-linking moiety” refers to a polyvalent, for example, divalent or trivalent, repeat unit which forms covalent bonds with one or more non-adjacent repeat units of the same polymer main chain or with one or more repeat units of different main chains.

The term “degree of crosslinking”, as used herein, refers to the fraction of repeat units that are capable of forming cross-link compared to the total number of repeat units in a polymer. Degree of crosslinking is generally expressed in mole percent with respect to the total number of repeat units in a polymer.

The phrase “number average molecular weight” refers to total weight of polymer divided by the total number of molecules. The number average molecular weight is the common average of the molecular weights of the individual polymer molecules. It is determined by measuring the molecular weight of n polymer molecules, summing the weights, and dividing by n.

In some embodiments, the polymers disclosed herein are ionomers. As used herein, the term “ionomer” refers to a polymer composed of both electrically neutral repeat units and repeat units comprising charged moieties (i.e., cations or anions) covalently bonded to the polymer backbone as pendant groups.

In some embodiments, the polymers provided herein are polyelectrolytes. As used herein, the term “polyelectrolyte” refers to polymer refers to a polymer which under a particular set of conditions has a net positive or negative charge due to the presence of charged repeat units. In some embodiments, a polyelectrolyte is or comprises a polycation; in some embodiments, a polyelectrolyte is or comprises a polyanion. Polycations have a net positive charge and polyanions have a net negative charge. The net charge of a given polyelectrolyte may depend on the surrounding chemical conditions, e.g., on the pH. As used herein, “ion exchange capacity” refers to the total number of active sites or functional groups responsible for ion exchange in a polyelectrolyte. Ion exchange capacity for a hydroxide-exchanging polyelectrolyte can be calculated according to Equation 1 based on the experimentally determined number of hydroxide ions that have been exchanged within the polymer. For polyelectrolyte-containing composite membranes ion accessibility is measured instead and calculated according to Equation 2, because the mass of the sample is a sum of the dry weight of the support plus the polymer.

$$IEC = \frac{meq {OH}^{-}}{Dry weight of polymer} \qquad Equation 1$$

$$IA = \frac{meq {OH}^{-}}{Dry weight of support + polymer} \qquad Equation 2$$

As used herein, “ionic conductivity” refers to the ability of the material, such as a polyelectrolyte, promote the movement of an ion through the material. For example, through-plane ionic conductivity of a polyelectrolyte membrane can be calculated based on the bulk resistance (R), the membrane active area (L), and the membrane thickness (A) according to Equation 3.

$$σ = \frac{L}{A \times R} \qquad Equation 3$$

As used herein, “porosity” refers to a fraction of the empty volume compared the total volume of the material. Porosity is a measureless value between 0 and 1, or as a percentage between 0% and 100%.

As used herein, the term “void space” or “void volume” refers to porosity of a composite that comprises a porous material impregnated with the polymer. Void space is different form the porosity of the porous material, since some of the pore volume of the porous material is taken up by the polymer disposed within the pore system of the material. A void space can be about 1%, about 2.5%, about 5%, about 7.5%, about 10%, about 12.5%, about 15%, about 17.5%, about 20%, about 25%, about 30%, about 35%, about 40%, about 45%, or about 50%.

As used herein, the term “polyolefin” refers to a polymer produced by polymerization of organic molecules containing a carbon-carbon double bond. The backbone of a polyolefin contains a saturated chain of carbon-carbon bonds. In some embodiments, the carbon atoms in the backbone of a polyolefin can be substituted with hydrocarbyl groups. For example, the carbon atoms in the backbone of a polyolefin can be substituted with alkyl, cycloalkyl, or aryl groups. In some embodiments, the carbon atoms in the backbone of a polyolefin can be substituted with halogens, such as fluorine.

As used herein, “perfluorinated polyolefin” refers to a polyolefin in which all hydrogen atoms have been substituted with fluorines.

As used herein, “inorganic material” refers to a material that does not contain chains of carbon-carbon bonds, except for elementary carbon allotropes, such as graphite, graphene, diamond, or carbon nanotubes, which are included in inorganic materials. Examples of inorganic materials include glass, ceramic materials, and metal oxides such as TiO₂, Al₂O₃, ZnO.

The term “ceramic material”, as used herein, refers to a crystalline or amorphous oxide, nitride or carbide of a metallic or non-metallic element. Ceramic materials are generally hard, brittle, heat-resistant and corrosion-resistant. Examples of ceramic materials include SiC, Si₃N₄, TiC, ZnO, ZrO₂, Al₂O₃, and MgO.

The term “current collector”, as used herein, refers to the electrical conductor between the electrode and external circuits in an electrochemical device such as a battery cell.

In some embodiments, the reinforcement material comprises a polymer, an inorganic material, or a combination thereof. For example, the reinforcement material comprises a polyolefin, a polyphenylene, a polyester, a polyamide, or a polysulfone. For example, the reinforcement material comprises a polyolefin such as polyethylene or polypropylene. For example, the reinforcement material comprises a perfluorinated polyolefin, such as polytetrafluoroethylene. For example, the reinforcement material comprises a polyimide, a polybenzimidazole, a polyphenylsulfone, a polyphenyl ether, polytetrafluoroethylene, cellulose nitrate, cellulose diacetate, cellulose triacetate, polypropylene, polyethylene, polyvinylidene fluoride, poly(phenylene sulfide), polyvinyl chloride, polystyrene, poly(methyl methacrylate), polyacrylonitrile, polyetheretherketone, polycarbonate, polyvinyltrimethylsilane, polytrimethylsilylpropyne, poly(ether imide), poly(ether sulfone), polyoxadiazole, poly(phenylene sulfide), or poly(phenylene oxide), or a combination or copolymer thereof. The composite material can comprise polyethylene, polypropylene, polytetrafluoroethylene, polyvinyl chloride, or polyvynyldifluoroethylene. Alternatively or additionally, the reinforcement material comprises fiberglass or a ceramic material.

In some embodiments, the composite material is an admixture of the reinforcement material and the polyelectrolyte. Alternatively or additionally, the reinforcement is a first layer; the electrolyte is a second layer; and the first layer is in contact with at least one second layer. Alternatively or additionally, the reinforcement is a porous material; and the porous material is impregnated with the electrolyte.

In some embodiments, the reinforcement is a porous material and the porous material has from about 40% to about 90% porosity, such as about 40%, about 45%, about 50%, about 55%, about 60%, about 65%, about 70%, about 75%, about 80%, about 85%, or about 90% porosity. For example, the porous material has from about 70% to about 85% porosity, such as about 73% porosity.

In some embodiments, the reinforcement is a porous material and an average size of pores of the porous material is from about 50 nm to about 500 μm, such as about 50 nm, about 100 nm, about 200 nm, about 300 nm, about 400 nm, about 500 nm, about 600 nm, about 700 nm, about 800 nm, about 900 nm, about 1 μm, about 1 μm, about 1 μm, about 10 μm, about 25 μm, about 50 μm, about 100 μm, about 150 μm, about 200 μm, about 250 μm, about 300 μm, about 350 μm, about 400 μm, about 450 μm, or about 500 μm. For example, the average size of the pores is from about 100 nm to about 10 μm, such as from about 300 nm to about 1 μm. For example, the average size of the pores is about 450 nm.

In some embodiments, the composite material is a film having a thickness from about 1 μm to about 300 μm, such as about 1 μm, about 5 μm, about 10 μm, about 20 μm, about 30 μm, about 40 μm, about 50 μm, about 60 μm, about 70 μm, about 80 μm, about 90 μm, about 100 μm, about 120 μm, about 140 μm, about 160 μm, about 180 μm, about 200 μm, about 220 μm, about 240 μm, about 260 μm, about 280 μm, or about 300 μm. For example, the composite material is a film having a thickness from about 25 μm to about 75 μm, such as about 50 μm.

In a first embodiment, the present invention is a compound represented by structural formula (I):

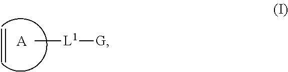

wherein:
the moiety represented by

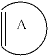

is a C₇₋₈ cycloalkenyl or a 7 to 12-membered heterocycloalkenyl,
L¹ is selected from a (O—C₁₋₁₂ alkylene) k, (C₁₋₁₂ alkylene-O) k, C₁₋₁₂ alkylene, C₆₋₁₂ arylene, C₆₋₁₂ arylene-C₁₋₁₂ alkylene, C₁₋₁₂ alkylene-C₆₋₁₂ arylene, C₁₋₁₂ alkylene-O—C₁₋₁₂ alkylene, C₁₋₁₂ alkylene-NH—C₁₋₁₂ alkylene, C₁₋₁₂ alkylene-N(C₁₋₁₂ alkyl)-C₁₋₁₂ alkylene, (NH—C₁₋₁₂ alkylene) k, (C₁₋₁₂ alkylene-NH) k;
the moiety represented by G is selected from a moiety represented by any one of the following structural formulas;

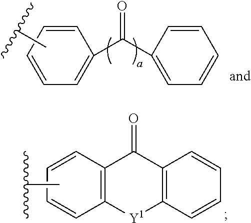

[symbol] is a point of attachment of the moiety represented by G to L¹;
k is an integer between 1 and 6;
a is 1 or 2;
Y¹ is —C(═O)—, O, S, NH, N(C₁₋₁₂ alkyl), or a bond; and
and further wherein:
each C₁₋₁₂ alkylene, C₆₋₁₂ arylene, C₁₋₁₂ alkyl, C₇₋₈cycloalkenyl, and 7 to 12-membered heterocycloalkenyl is independently optionally substituted with 1 to 6 substituents selected from the group consisting of F, Cl, Br, OH, NH₂, C₁₋₁₂ alkyl, C₆₋₁₂ aryl a C₁₋₁₂ haloalkyl, C₁₋₁₂ alkoxy, C₆₋₁₂ aryl, C₆₋₁₂ aryloxy, NH(C₁₋₁₂ alkyl), N(C₁₋₁₂ alkyl)₂, C(O)O(C₁₋₁₂ alkyl), and C(O)NH(C₁₋₁₂ alkyl).

In a first aspect of the first embodiment, the moiety represented by

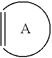

is a C₇₋₈cycloalkenyl, the moiety represented by G is a moiety represented by the following structural formula:

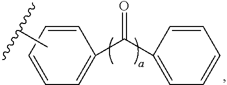

and L¹ is C₁₋₁₂ alkylene, (O—C₁₋₁₂ alkylene) k or (C₁₋₁₂ alkylene-O)ₖ.

In a second aspect of the first embodiment, the moiety represented by

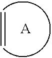

is a 7 to 12-membered heterocycloalkenyl, the moiety represented by G is a moiety represented by the following structural formula:

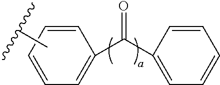

and L¹ is C₁₋₁₂ alkylene, (O—C₁₋₁₂ alkylene)ₖ or (C₁₋₁₂ alkylene-O)ₖ. The remainder of features and example features of the second aspect is as described above with respect to the first aspect of the first embodiment.

In a third aspect of the first embodiment, a is 1 or 2. For example, a is 1. Alternatively, a is 2. The remainder of features and example features of the third aspect is as described above with respect to the first through second aspects of the first embodiment.

In a fourth aspect of the first embodiment, the moiety represented by

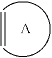

is a C₇₋₈cycloalkenyl, the moiety represented by G is a moiety represented by the following structural formula:

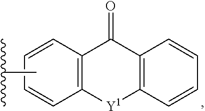

and L¹ is C₁₋₁₂ alkylene, (O—C₁₋₁₂ alkylene)ₖ or (C₁₋₁₂ alkylene-O)ₖ. The remainder of features and example features of the fourth aspect is as described above with respect to the first through third aspects of the first embodiment.

In a fifth aspect of the first embodiment, the moiety represented by

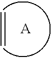

is a 7 to 12-membered heterocycloalkenyl, the moiety represented by G is a moiety represented by the following structural formula:

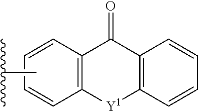

and L¹ is C₁₋₁₂ alkylene, (O—C₁₋₁₂ alkylene)ₖ or (C₁₋₁₂ alkylene-O)ₖ. The remainder of features and example features of the fifth aspect is as described above with respect to the first through fourth aspects of the first embodiment.

In a sixth aspect of the first embodiment, Y¹ is —C(═O)—, O, S. For example, Y is —C(═O)—. For example, Y is O. For example, Y is S. The remainder of features and example features of the sixth aspect is as described above with respect to the first through fifth aspects of the first embodiment.

In a seventh aspect of the first embodiment, Y¹ is NH or a N(C₁₋₁₂ alkyl). For example, Y is NH. For example, Y is N(C₁₋₁₂ alkyl). The remainder of features and example features of the seventh aspect is as described above with respect to the first through sixth aspects of the first embodiment.

In an eighth aspect of the first embodiment, Y¹ is a bond. The remainder of features and example features of the eighth aspect is as described above with respect to the first through seventh aspects of the first embodiment.

In a ninth aspect of the first embodiment, the moiety represented by

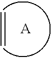

is a moiety represented by any one of the following structural formulas:

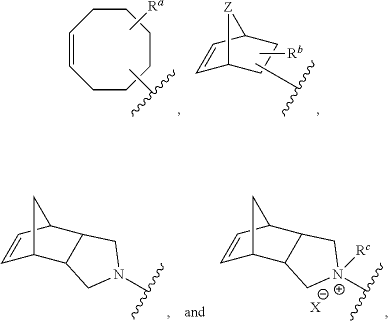

wherein:
R^{a}, R^{b}, and R^{c} each independently is H or a C₁₋₁₂ alkyl;
Z is selected from CH₂, O, NH, and N(C₁₋₁₂ alkyl); and
X⁻ is selected from F⁻, Cl⁻, Br⁻, OH⁻, NO⁻₂, CN⁻, HCO₃⁻, CO₃²⁻, PF⁻, BF₄⁻, a C₁₋₁₂ carboxylate, and a C₁₋₁₂ alkoxide. The remainder of features and example features of the ninth aspect is as described above with respect to the first through eighth aspects of the first embodiment.

In a tenth aspect of the first embodiment, the moiety represented by

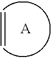

is

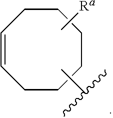

For example, the moiety represented by

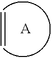

is

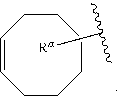

The remainder of features and example features of the tenth aspect is as described above with respect to the first through ninth aspects of the first embodiment.

In an eleventh aspect of the first embodiment, R^{a} is H. The remainder of features and example features of the eleventh aspect is as described above with respect to the first through ninth through tenth aspects of the first embodiment.

In a twelfth aspect of the first embodiment, R^{a} is a C₁₋₃ alkyl. For example, R^{a} is methyl. The remainder of features and example features of the twelfth aspect is as described above with respect to the first through eleventh aspects of the first embodiment.

In a thirteenth aspect of the first embodiment, the moiety represented by

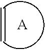

is

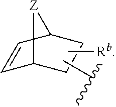

The remainder of features and example features of the thirteenth aspect is as described above with respect to the first through ninth aspects of the first embodiment.

In a fourteenth aspect of the first embodiment, Z is CH₂. Alternatively, Z is O, NH, or N(C₁₋₁₂ alkyl). The remainder of features and example features of the fourteenth aspect is as described above with respect to the first through thirteenth aspects of the first embodiment.

In a fifteenth aspect of the first embodiment, R^{b} is H or Me. For example, R^{b} is methyl. Alternatively, R^{b} is H. The remainder of features and example features of the fifteenth aspect is as described above with respect to the first through fourteenth aspects of the first embodiment.

In a sixteenth aspect of the first embodiment, the moiety represented by

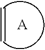

is

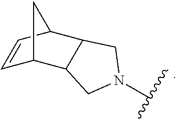 [SMILES: *N1CC2C3C=CC(C3)C2C1]

The remainder of features and example features of the sixteenth aspect is as described above with respect to the first through fifteenth aspects of the first embodiment.

In a seventeenth aspect of the first embodiment, the moiety represented by

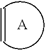

is

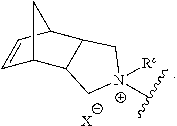

The remainder of features and example features of the seventeenth aspect is as described above with respect to the first through sixteenth aspects of the first embodiment.

In an eighteenth aspect of the first embodiment, R^{c} is a C₁₋₃ alkyl. For example, R^{c} is methyl. The remainder of features and example features of the eighteenth aspect is as described above with respect to the seventeenth aspect of the first embodiment.

In a nineteenth aspect of the first embodiment, L¹ is (O—C₁₋₁₂ alkylene)ₖ or (C₁₋₁₂ alkylene-O)ₖ. For example, L¹ is —CH₂O— or —OCH₂—. The remainder of features and example features of the nineteenth aspect is as described above with respect to the first through eighteenth aspects of the first embodiment.

In a twentieth aspect of the first embodiment, k is 1, 2, 3, 4, 5, or 6. For example, k is 1. The remainder of features and example features of the twentieth aspect is as described above with respect to the first through nineteenth aspects of the first embodiment.

In a twenty-first aspect of the first embodiment, L¹ is a C₁₋₁₂ alkylene. For example, L¹ is C₁ alkylene, C₂ alkylene, C₃ alkylene, C₄ alkylene, C₅ alkylene, C₆ alkylene, C₇ alkylene, C₈ alkylene, C₉ alkylene, C₁₀ alkylene, C₁₁ alkylene, or C₁₂ alkylene. The remainder of features and example features of the twenty-first aspect is as described above with respect to the first through twentieth aspects of the first embodiment.

In a twenty-third aspect of the first embodiment, the compound is selected from:

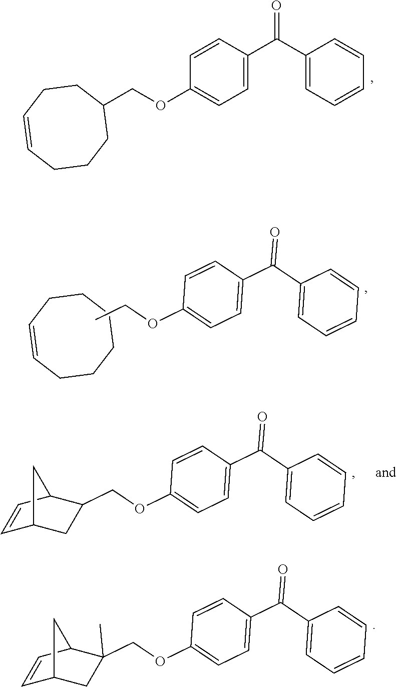

In a second embodiment, the present invention is a polymer, comprising: a plurality of first repeat units represented by structural formula (II):

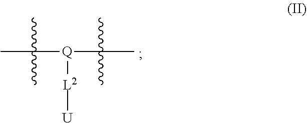

and

a plurality of second repeat units represented by structural formula (III):

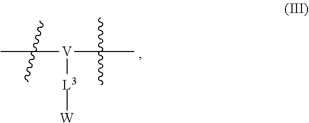

wherein:
Q is a moiety represented by one of the following structural formulas:

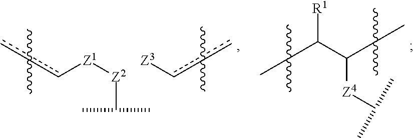

U is a moiety represented by one of the following structural formulas:

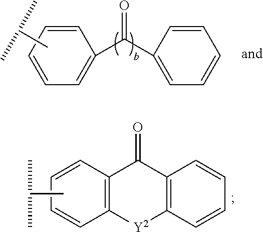

V is a moiety represented by one of the following structural formulas:

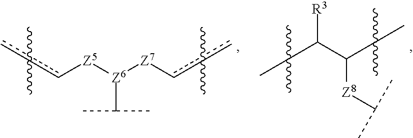

W is a C₁₋₁₂ alkyl or a moiety represented by one of the structural formulas selected from:

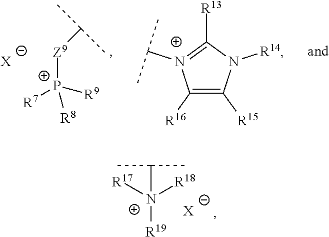

[symbol] is a point of attachment to adjacent repeat units of the polymer;
[symbol] is a point of attachment to L²;
[symbol] is a point of attachment to L³
and further wherein:
[symbol] is a double bond or a single bond;
Z¹, Z³, Z⁵, and Z⁷ each independently is a C₁₋₃ alkylene or a bond;
Z² is selected from —CHR⁵—, a C₅₋₁₂ cycloalkylene, and a 5 to 16-membered heterocyclylene;
Z⁴ is selected from a bond, a C₆₋₁₂ arylene, and CR², in which case R¹ and R² together with the carbon atoms to which they are attached form a C₅₋₁₂ cycloalkyl or a 5 to 12-membered heterocyclyl;
Z⁶ is selected from —CHR⁶—, a C₅₋₁₂ cycloalkylene, and a 5 to 16-membered heterocyclylene;
Z⁸ is selected from a bond, a C₆₋₁₂ arylene, and CR⁴, in which case R³ and R⁴ together with the carbon atoms to which they are attached form a C₅₋₁₂ cycloalkyl or a 5 to 12 membered heterocyclyl;
Z⁹ is NR¹⁰ or a bond;
R¹, if present, and R³, if present, each independently is selected from H, a C₁₋₁₂ alkyl, and C₆₋₁₂ aryl;
R⁵ and R⁶ each independently is H or a C₁₋₁₂ alkyl;
R⁷, R⁸, and R⁹ each independently is selected from NR¹¹R¹², a C₆₋₁₂ aryl, and 5 to 12-membered heterocyclyl;
R¹⁰ is a C₁₋₁₂ alkyl;
R¹¹ and R¹² each independently is C₁₋₁₂ alkyl or a C₃₋₁₂ cycloalkyl, or R¹¹ and R¹² together with the nitrogen atom to which they are attached form a 5 to 12-membered heterocyclyl;
R¹³ is selected from a C₁₋₁₂ alkyl, C₆₋₁₂ aryl, and 5 to 12-membered heterocyclyl;
R¹⁴ is a C₁₋₁₂ alkyl or a C₃₋₁₂ cycloalkyl;
R¹⁵ and R¹⁶ each independently is selected from C₁₋₁₂ alkyl, C₆₋₁₂ aryl, and 5 to 12-membered heterocyclyl; or R¹⁵ and R¹⁶ together with the carbon atoms to which they are attached form a C₆₋₁₂ aryl or a 5 to 12-membered heterocyclyl; and
R¹⁷, R¹⁸, and R¹⁹ each independently is a C₁₋₁₂ alkyl or a C₃₋₁₂ cycloalkyl; or
R¹⁸ and R¹⁹ together with the nitrogen atom to which they are attached form a 5 to 12-membered heterocyclyl and (i) R¹⁷ is a C₁₋₁₂ alkyl or a C₃₋₁₂ cycloalkyl, or (ii) R¹⁷ and at least one atom of L³, if present, together with the nitrogen atom to which L³ and R¹⁷ are attached form a 5- to 12-membered heterocyclyl; or
R¹⁷, R¹⁸, and R¹⁹ together with the nitrogen atom to which they are attached form a bicyclic 5 to 12-membered heterocyclyl; and
L² is selected from a C₁₋₁₂ alkylene, C₆₋₁₂ arylene, C₆₋₁₂ arylene-C₁₋₁₂ alkylene, C₁₋₁₂ alkylene-C₆₋₁₂ arylene, C₁₋₁₂ alkylene-O—C₁₋₁₂ alkylene, (O—C₁₋₁₂ alkylene) m, (C₁₋₁₂ alkylene-O) m, C₁₋₁₂ alkylene-NH—C₁₋₁₂ alkylene, C₁₋₁₂ alkylene-N(C₁₋₁₂ alkyl)-C₁₋₁₂ alkylene, (NH—C₁₋₁₂ alkylene)ₘ, and (C₁₋₁₂ alkylene-NH)ₘ; 1L³ is selected from a C₁₋₁₂ alkylene, C₆₋₁₂ arylene, C₆₋₁₂ arylene-C₁₋₁₂ alkylene, C₁₋₁₂ alkylene-C₆₋₁₂ arylene, C₁₋₁₂ alkylene-O—C₁₋₁₂ alkylene, (O—C₁₋₁₂ alkylene) n, (C₁₋₁₂ alkylene-O) n, C₁₋₁₂ alkylene-NH—C₁₋₁₂ alkylene, C₁₋₁₂ alkylene-N(C₁₋₁₂ alkyl)-C₁₋₁₂ alkylene, (NH—C₁₋₁₂ alkylene) n, (C₁₋₁₂ alkylene-NH) n, and a bond;
m is an integer between 1 and 6;
n is an integer between 1 and 6;
b is 1 or 2;
Y² is selected from —C(═O)—, O, S, NH, N(C₁₋₁₂ alkyl), and a bond;
X⁻ is selected from F⁻, Cl⁻, Br⁻, OH⁻, NO⁻₂, CN⁻, HCO₃⁻, CO₃²⁻, PF⁻, BF₄⁻, a C₁₋₁₂ carboxylate and a C₁₋₁₂ alkoxide;
provided that W is C₁₋₁₂ alkyl only when V is a cationic moiety,
   and wherein:
each C₁₋₁₂ alkylene, C₆₋₁₂ arylene, C₅₋₁₂ cycloalkylene, 5 to 16-membered heterocyclylene, C₁₋₁₂ alkyl, C₃₋₁₂ cycloalkyl, C₅₋₁₂ cycloalkyl, C₆₋₁₂ aryl, and 5 to 12-membered heterocyclyl is independently optionally substituted with 1 to 6 substituents independently selected from the group consisting of F, Cl, Br, OH, NH₂, oxo, a C₁₋₁₂ alkyl, C₆₋₁₂ aryl, C₁₋₁₂ haloalkyl, C₁₋₁₂ alkoxy, C₆₋₁₂ aryl, C₆₋₁₂ aryloxy, NH(C₁₋₁₂ alkyl), N(C₁₋₁₂ alkyl)₂, C(O)O(C₁₋₁₂ alkyl), and C(O)NH(C₁₋₁₂ alkyl).

In a first aspect of the second embodiment, U is a moiety represented by the following structural formula:

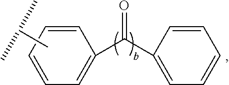

and W is a moiety represented by the following structural formula:

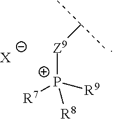

In a second aspect of the second embodiment, U is a moiety represented by the following structural formula:

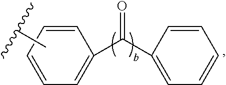

and W is a moiety represented by the following structural formula:

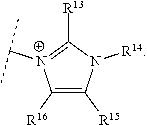

The remainder of features and example features of the second aspect is as described above with respect to the first aspect of the second embodiment.

In a third aspect of the second embodiment, U is a moiety represented by the following structural formula:

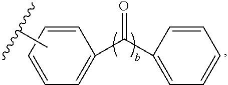

and W is a moiety represented by the following structural formula:

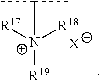

The remainder of features and example features of the third aspect is as described above with respect to the first through second aspects of the second embodiment.

In a fourth aspect of the second embodiment, U is a moiety represented by the following structural formula:

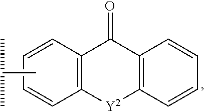

and W is a moiety represented by one of the following structural formulas:

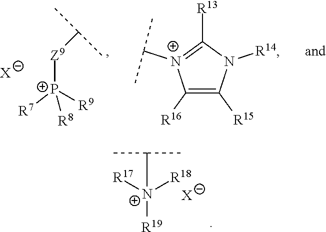

The remainder of features and example features of the fourth aspect is as described above with respect to the first through third aspects of the second embodiment.

In a fifth aspect of the second embodiment, Q is a moiety represented by the following structural formula:

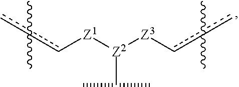

The remainder of features and example features of the fifth aspect is as described above with respect to the first through fourth aspects of the second embodiment.

In a sixth aspect of the second embodiment, Q is a moiety represented by the following structural formula:

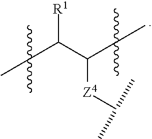

The remainder of features and example features of the sixth aspect is as described above with respect to the first through fifth aspects of the second embodiment.

In a seventh aspect of the second embodiment, V is a moiety represented by the following structural formula:

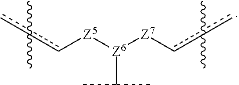

The remainder of features and example features of the seventh aspect is as described above with respect to the first through sixth aspects of the second embodiment.

In an eighth aspect of the second embodiment, V is a moiety represented by the following structural formula:

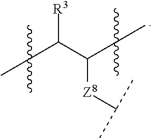

The remainder of features and example features of the eighth aspect is as described above with respect to the first through seventh aspects of the second embodiment.

In a ninth aspect of the second embodiment, Q is a moiety represented by the following structural formula:

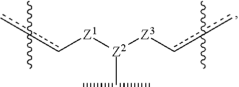

is a moiety represented by the following structural formula

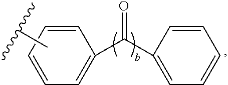

is a moiety represented by the following structural formula:

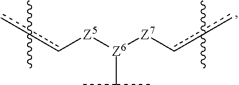

and W is a moiety represented by the following structural formula:

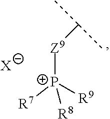

The remainder of features and example features of the ninth aspect is as described above with respect to the first through eighth aspects of the second embodiment.

In a tenth aspect of the second embodiment, Q is a moiety represented by the following structural formula:

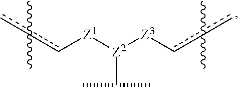

is a moiety represented by the following structural formula:

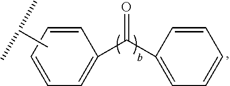

is a moiety represented by the following structural formula:

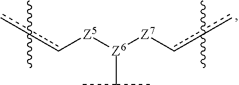

and W is a moiety represented by the following structural formula:

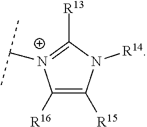

The remainder of features and example features of the tenth aspect is as described above with respect to the first through ninth aspects of the second embodiment.

In an eleventh aspect of the second embodiment, Q is a moiety represented by the following structural formula:

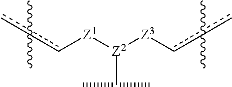

is a moiety represented by the following structural formula:

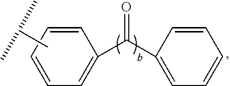

is a moiety represented by the following structural formula:

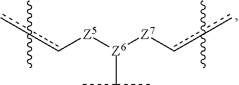

and W is a moiety represented by the following structural formula:

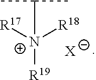

The remainder of features and example features of the eleventh aspect is as described above with respect to the first through tenth aspects of the second embodiment.

In a twelfth aspect of the second embodiment, Q is a moiety represented by any one of the following structural formulas:

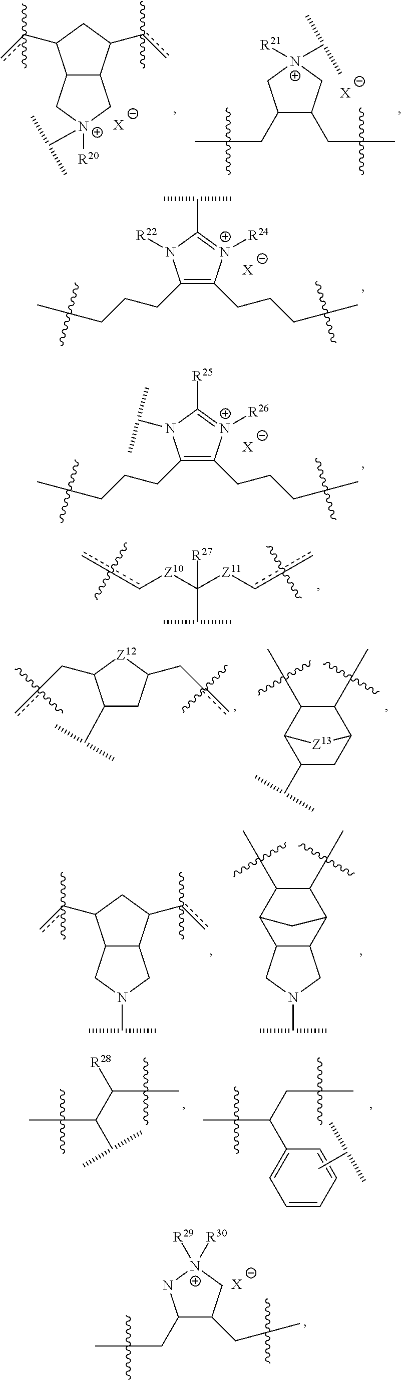

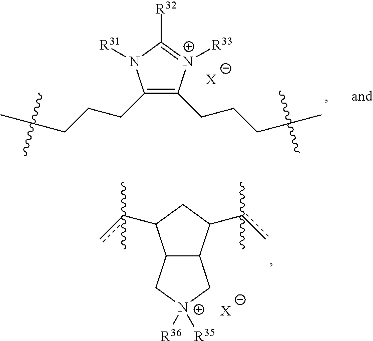

wherein:
R²⁰, R²¹, R²², R²⁴, R²⁶, R²⁹, R³⁰, R³¹, and R³³ each independently is a C₁₋₁₂ alkyl;
R²⁵ and R³² each independently is a C₆₋₁₂ aryl;
R²⁷ is H or a C₁₋₁₂ alkyl;
R²⁸ is H, a C₁₋₁₂ alkyl, or a C₆₋₁₂ aryl;
R³⁶ and R³⁵ each independently is a C₁₋₁₂ alkyl, or R³⁶ and R³⁵ together with the nitrogen atom to which they are attached form a 5- to 12-membered heterocyclyl;
Z¹⁰ and Z¹¹ each independently is a C₁₋₃ alkylene or a bond; and
Z¹² and Z¹³ each independently is selected from CH₂, O, NH, and N(C₁₋₁₂ alkyl). The remainder of features and example features of the twelfth aspect is as described above with respect to the first through eleventh aspects of the second embodiment.

In a thirteenth aspect of the second embodiment, Q is a moiety represented by any one of the following structural formulas:

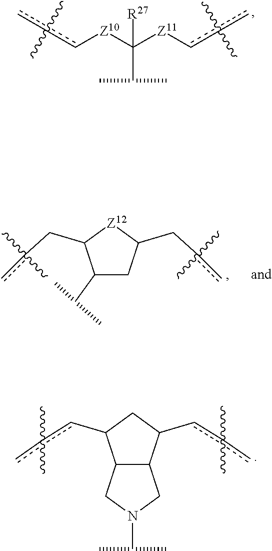

The remainder of features and example features of the thirteenth aspect is as described above with respect to the first through twelfth aspects of the second embodiment.

In a fourteenth aspect of the second embodiment, Q is a moiety represented by the following structural formula:

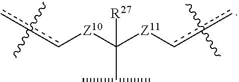

For example, Q is a moiety represented by the following structural formula:

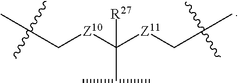

Alternatively, Q is a moiety represented by the following structural formula:

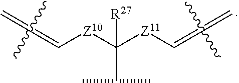

The remainder of features and example features of the fourteenth aspect is as described above with respect to the first through thirteenth aspects of the second embodiment.

In a fifteenth aspect of the second embodiment, Z¹⁰ and Z¹¹ each independently is a C₁₋₃ alkylene. For example, Z¹⁰ is C₂ alkylene and Z¹¹ is C₃ alkylene. The remainder of features and example features of the fifteenth aspect is as described above with respect to the first through fourteenth aspects of the second embodiment.

In a sixteenth aspect of the second embodiment, R²⁷ is H or methyl. The remainder of features and example features of the sixteenth aspect is as described above with respect to the first through fifteenth aspects of the second embodiment.

In a seventeenth aspect of the second embodiment, Q is a moiety represented by the following structural formula:

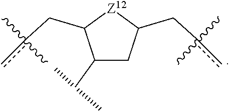

For example, Q is a moiety represented by the following structural formula:

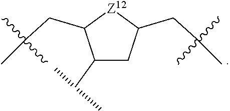

Alternatively, Q is a moiety represented by the following structural formula:

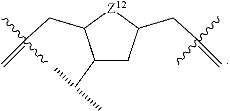

The remainder of features and example features of the seventeenth aspect is as described above with respect to the first through sixteenth aspects of the second embodiment.

In an eighteenth aspect of the second embodiment, Z¹² is CH₂, O, NH or N(C₁₋₁₂ alkyl). For example, Z¹² is CH₂ or O. Alternatively, Z¹² is NH or N(C₁₋₁₂ alkyl). The remainder of features and example features of the eighteenth aspect is as described above with respect to the first through seventeenth aspects of the second embodiment.

In a nineteenth aspect of the second embodiment, Q is a moiety represented by any one of the following structural formulas:

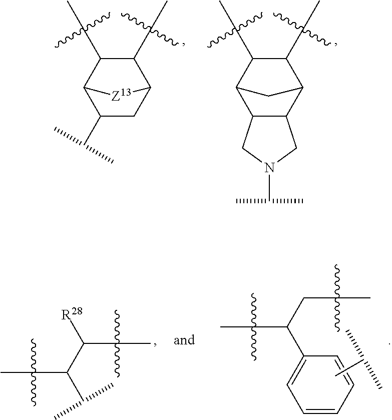

The remainder of features and example features of the nineteenth aspect is as described above with respect to the first through eighteenth aspects of the second embodiment.

In a twentieth aspect of the second embodiment, Q

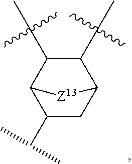

The remainder of features and example features of the twentieth aspect is as described above with respect to the first through nineteenth aspects of the second embodiment.

In a twenty-first aspect of the second embodiment, Z¹³ is CH₂, O, NH, or N(C₁₋₁₂ alkyl). For example, Z¹³ is CH₂ or O. Alternatively, Z¹³ is NH or N(C₁₋₁₂ alkyl). The remainder of features and example features of the twenty-first aspect is as described above with respect to the first through twentieth aspects of the second embodiment.

In a twenty-second aspect of the second embodiment, Q is a moiety represented by the following structural formula:

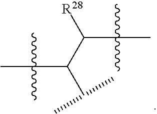

The remainder of features and example features of the twenty-second aspect is as described above with respect to the first through twenty-first aspects of the second embodiment.

In a twenty-third aspect of the second embodiment, R²⁸ is H or methyl. For example, R²⁸ is H. Alternatively, R²⁸ is methyl. The remainder of features and example features of the twenty-third aspect is as described above with respect to the first through twenty-second aspects of the second embodiment.

In a twenty-fourth aspect of the second embodiment, Q is a moiety represented by the following structural formula:

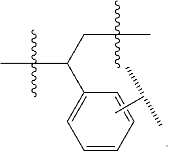

The remainder of features and example features of the twenty-fourth aspect is as described above with respect to the first through twenty-third aspects of the second embodiment.

In a twenty-fifth aspect of the second embodiment, V is a moiety represented by any one of the following structural formulas:

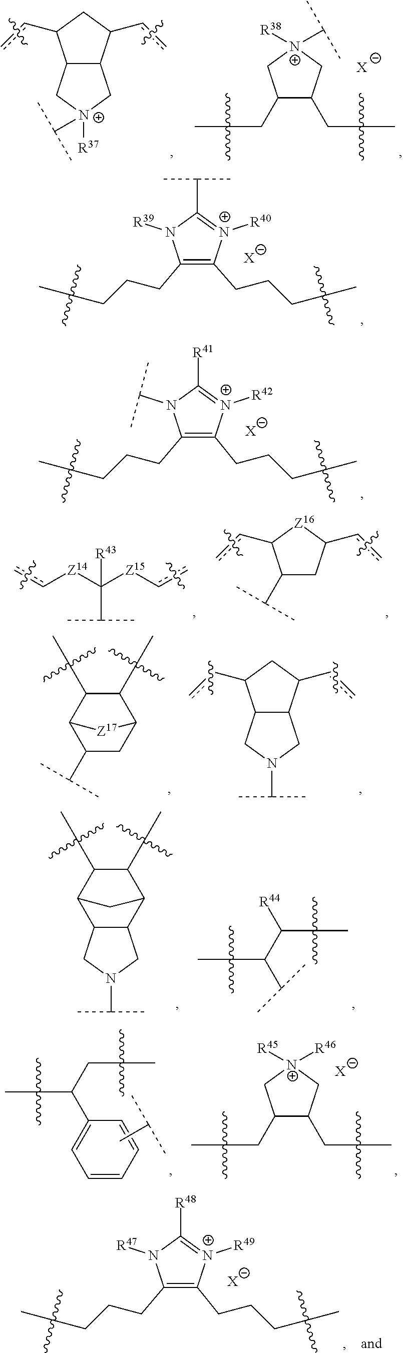

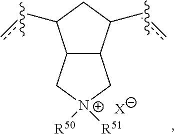

wherein:
R³⁷, R³⁸, R³⁹, R⁴⁰, R⁴², R⁴⁵, R⁴⁶, R⁴⁷, and R⁴⁹ each independently is a C₁₋₁₂ alkyl;
R⁴¹ and R⁴⁸ each independently is a C₆₋₁₂ aryl;
R⁴³ is H or a C₁₋₁₂ alkyl;
R⁴⁴ is H, a C₁₋₁₂ alkyl, or a C₆₋₁₂ aryl;
R⁵⁰ and R⁵¹ each independently is a C₁₋₁₂ alkyl, or R³⁶ and R³⁵ together with the nitrogen atom to which they are attached form a 5- to 12-membered heterocyclyl;
Z¹⁴ and Z¹⁵ each independently is a C₁₋₃ alkylene or a bond; and
Z¹⁶ and Z¹⁷ each independently is selected from CH₂, O, NH, and N(C₁₋₁₂ alkyl). The remainder of features and example features of the twenty-fifth aspect is as described above with respect to the first through twenty-fourth aspects of the second embodiment.

In a twenty-sixth aspect of the second embodiment, V is a moiety represented by any one of the following structural formulas:

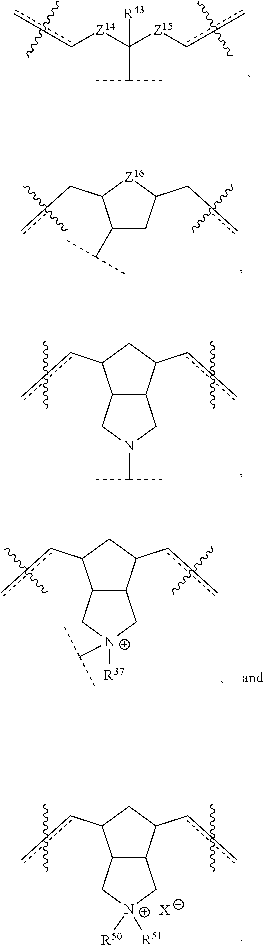

The remainder of features and example features of the twenty-sixth aspect is as described above with respect to the first through twenty-fifth aspects of the second embodiment.

In a twenty-seventh aspect of the second embodiment, V is a moiety represented by the following structural formula:

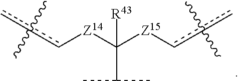

For example, V is a moiety represented by the following structural formula:

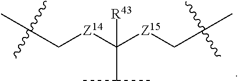

Alternatively, V is a moiety represented by the following structural formula:

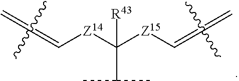

The remainder of features and example features of the twenty-seventh aspect is as described above with respect to the first through twenty-sixth aspects of the second embodiment.

In a twenty-eighth aspect of the second embodiment, Z¹⁴ and Z¹⁵ each independently is a C₁₋₃ alkylene. For example, Z¹⁴ is C₂ alkylene and Z¹⁵ is C₃ alkylene. The remainder of features and example features of the twenty-eighth aspect is as described above with respect to the first through twenty-seventh aspects of the second embodiment.

In a twenty-ninth aspect of the second embodiment, R⁴³ is H or methyl. For example, R⁴³ is H. Alternatively, R⁴³ is methyl. The remainder of features and example features of the twenty-ninth aspect is as described above with respect to the first through twenty-eighth aspects of the second embodiment.

In a thirtieth aspect of the second embodiment, V is a moiety represented by the following structural formula:

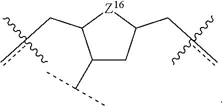

For example, V is a moiety represented by the following structural formula:

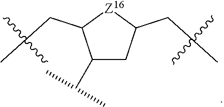

Alternatively, V is a moiety represented by the following structural formula:

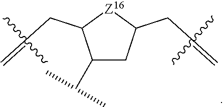

The remainder of features and example features of the thirtieth aspect is as described above with respect to the first through twenty-ninth aspects of the second embodiment.

In a thirty-first aspect of the second embodiment, Z¹⁶ is CH₂, O, NH or N(C₁₋₁₂ alkyl). For example, Z¹⁶ is CH₂ or O. Alternatively, Z¹⁶ is NH or N(C₁₋₁₂ alkyl). The remainder of features and example features of the thirty-first aspect is as described above with respect to the first through thirtieth aspects of the second embodiment.

In a thirty-second aspect of the second embodiment, V is a moiety represented by any one of the following structural formulas:

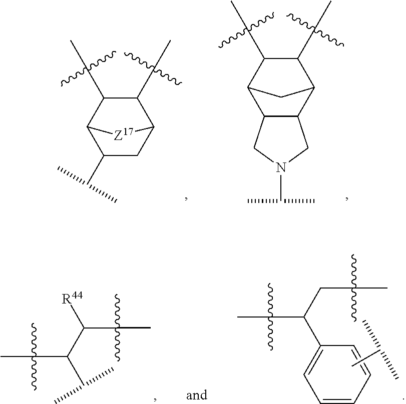

The remainder of features and example features of the thirty-second aspect is as described above with respect to the first through thirty-first aspects of the second embodiment.

In a thirty-third aspect of the second embodiment, V is a moiety represented by the following structural formula:

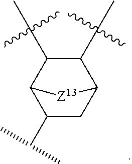

The remainder of features and example features of the thirty-third aspect is as described above with respect to the first through thirty-second aspects of the second embodiment.

In a thirty-fourth aspect of the second embodiment, Z¹³ is CH₂, O, NH or N(C₁₋₁₂ alkyl). For example, Z¹³ is CH₂ or O. Alternatively, Z¹³ is NH or N(C₁₋₁₂ alkyl). The remainder of features and example features of the thirty-fourth aspect is as described above with respect to the first through thirty-third aspects of the second embodiment.

In a thirty-fifth aspect of the second embodiment, V is a moiety represented by the following structural formula

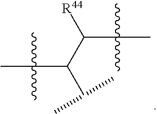

The remainder of features and example features of the thirty-fifth aspect is as described above with respect to the first through thirty-fourth aspects of the second embodiment.

In a thirty-sixth aspect of the second embodiment, R⁴⁴ is H or methyl. For example, R⁴⁴ is H. Alternatively, R⁴⁴ is methyl. The remainder of features and example features of the thirty-sixth aspect is as described above with respect to the first through thirty-fifth aspects of the second embodiment.

In a thirty-seventh aspect of the second embodiment, V is a moiety represented by the following structural formula:

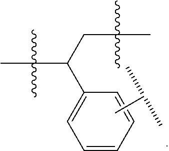

The remainder of features and example features of the thirty-seventh aspect is as described above with respect to the first through thirty-sixth aspects of the second embodiment.

In a thirty-eighth aspect of the second embodiment,

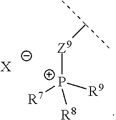

The remainder of features and example features of the thirty-eighth aspect is as described above with respect to the first through thirty-seventh aspects of the second embodiment.

In a thirty-ninth aspect of the second embodiment, Z⁹ is NR¹⁰; and R⁷, R⁸, and R⁹ each independently is NR¹¹R¹². The remainder of features and example features of the thirty-ninth aspect is as described above with respect to the first through thirty-eighth aspects of the second embodiment.

In a fortieth aspect of the second embodiment, Z⁹ is a bond and R⁷, R⁸, and R⁹ each independently is a C₆₋₁₂ aryl. For example, R⁷, R⁸, and R⁹ each is phenyl. The remainder of features and example features of the fortieth aspect is as described above with respect to the first through thirty-ninth aspects of the second embodiment.

In a forty-first aspect of the second embodiment, R¹¹ and R¹² each independently is C₁₋₁₂ alkyl or a C₃₋₁₂ cycloalkyl. For example, R¹¹ is a C₁₋₃ alkyl, and R¹² is a C₅₋₇ cycloalkyl or a C₁₋₃ alkyl. For example, R¹¹ and R¹² are each methyl; or R¹¹ is methyl and R¹² is isopropyl; or R¹¹ is cyclohexyl and R¹² is methyl. The remainder of features and example features of the forty-first aspect is as described above with respect to the first through fortieth aspects of the second embodiment.

In a forty-second aspect of the second embodiment, W is

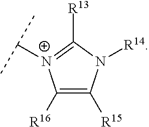

The remainder of features and example features of the forty-second aspect is as described above with respect to the first through forty-first aspects of the second embodiment.

In a forty-third aspect of the second embodiment, R¹³ is an unsubstituted C₆₋₁₂ aryl. For example, R¹³ is an unsubstituted phenyl. Alternatively, R¹³ is a C₆₋₁₂ aryl substituted with 1 to 3 substituents independently selected from a C₁₋₁₂ alkyl, C₁₋₁₂ alkoxy, and N(C₁₋₁₂ alkyl)₂. For example, R¹³ is a C₆₋₁₂ aryl substituted with 1 to 3 substituents independently selected from methyl, ethyl, propyl, isopropyl, methoxy, ethoxy, propoxy, isopropoxy, dimethylamino, or diethylamino, such as phenyl substituted with 1 to 3 substituents independently selected from methyl, ethyl, propyl, isopropyl, methoxy, ethoxy, propoxy, isopropoxy, dimethylamino, or diethylamino. The remainder of features and example features of the forty-third aspect is as described above with respect to the first through forty-second aspects of the second embodiment.

In a forty-fourth aspect of the second embodiment, R¹⁴ is a C₁₋₁₂ alkyl. For example, R¹⁴ is methyl, ethyl, propyl, isopropyl, butyl, sec-butyl, isobutyl, or tert-butyl. Alternatively, R¹⁴ is a C₃₋₈ cycloalkyl, such as cyclopropyl, cyclobutyl, cyclopentyl, or cyclohexyl. The remainder of features and example features of the forty-fourth aspect is as described above with respect to the first through forty-third aspects of the second embodiment.

In a forty-fifth aspect of the second embodiment, R¹⁵ and R¹⁶ each independently is a C₁₋₁₂ alkyl. For example, R¹⁵ and R¹⁶ each independently is methyl, ethyl, propyl, isopropyl, butyl, sec-butyl, isobutyl, or tert-butyl. For example, R¹⁵ and R¹⁶ each is methyl. The remainder of features and example features of the forty-fifth aspect is as described above with respect to the first through forty-fourth aspects of the second embodiment.

In a forty-sixth aspect of the second embodiment, R¹⁵ and R¹⁶ each independently is a C₆₋₁₂ aryl. For example, R¹⁵ and R¹⁶ are each phenyl. The remainder of features and example features of the forty-sixth aspect is as described above with respect to the first through forty-fifth aspects of the second embodiment.

In a forty-seventh aspect of the second embodiment, R¹⁵ and R¹⁶ together with the carbon atoms to which they are attached form a C₆₋₁₂ aryl. For example, R¹⁵ and R¹⁶ together with the carbon atoms to which they are attached form a C₆ aryl. The remainder of features and example features of the forty-seventh aspect is as described above with respect to the first through forty-sixth aspects of the second embodiment.

In a forty-eighth aspect of the second embodiment, W is

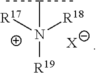

The remainder of features and example features of the forty-eighth aspect is as described above with respect to the first through forty-seventh aspects of the second embodiment.

In a forty-ninth aspect of the second embodiment, R¹⁷, R¹⁸, and R¹⁹ each independently is selected from a C₁₋₁₂ alkyl. For example, R¹⁷, R¹⁸, and R¹⁹ each is methyl. The remainder of features and example features of the forty-ninth aspect is as described above with respect to the first through forty-eighth aspects of the second embodiment.

In a fiftieth aspect of the second embodiment, R¹⁷ is a C₁₋₁₂ alkyl and R¹⁸, and R¹⁹ together with the nitrogen atom to which they are attached form a 5 to 12-membered heterocyclyl. The remainder of features and example features of the fiftieth aspect is as described above with respect to the first through forty-ninth aspects of the second embodiment.

In a fifty-first aspect of the second embodiment, R¹⁷, R¹⁸, and R¹⁹ together with the nitrogen atom to which they are attached form a bicyclic 5 to 12-membered heterocyclyl. The remainder of features and example features of the fifty-first aspect is as described above with respect to the first through fiftieth aspects of the second embodiment.

In a fifty-second aspect of the second embodiment, L² is a C₁₋₁₂ alkylene. For example, L² is C₁ alkylene, C₂ alkylene, C₃ alkylene, C₄ alkylene, C₅ alkylene, C₆ alkylene, C₇ alkylene, C₈ alkylene, C₉ alkylene, C₁₀ alkylene, C₁₁ alkylene, or C₁₂ alkylene. The remainder of features and example features of the fifty-second aspect is as described above with respect to the first through fifty-first aspects of the second embodiment.

In a fifty-third aspect of the second embodiment, L² is (O—C₁₋₁₂ alkylene)ₘ or (C₁₋₁₂ alkylene-O)ₘ. For example, L² is —CH₂O— or —OCH₂—. The remainder of features and example features of the fifty-third aspect is as described above with respect to the first through fifty-second aspects of the second embodiment.

In a fifty-fourth aspect of the second embodiment, m is 1, 2, 3, 4, 5, or 6. For example, m is 1. The remainder of features and example features of the fifty-fourth aspect is as described above with respect to the first through fifty-third aspects of the second embodiment.

In a fifty-fifth aspect of the second embodiment, L³ is a C₁₋₁₂ alkylene. For example, L³ is C₁ alkylene, C₂ alkylene, C₃ alkylene, C₄ alkylene, C₅ alkylene, C₆ alkylene, C₇ alkylene, C₈ alkylene, C₉ alkylene, C₁₀ alkylene, C₁₁ alkylene, or C₁₂ alkylene. For example, L³ is methylene. The remainder of features and example features of the fifty-fifth aspect is as described above with respect to the first through fifty-fourth aspects of the second embodiment.

In a fifty-sixth aspect of the second embodiment, L³ is (O—C₁₋₁₂ alkylene) n or (C₁₋₁₂ alkylene-0) n. For example, L³ is —CH₂O— or —OCH₂—. The remainder of features and example features of the fifty-sixth aspect is as described above with respect to the first through fifty-fifth aspects of the second embodiment.

In a fifty-seventh aspect of the second embodiment, n is 1, 2, 3, 4, 5, or 6. For example, n is 1. The remainder of features and example features of the fifty-seventh aspect is as described above with respect to the first through fifty-sixth aspects of the second embodiment.

In a fifty-eighth aspect of the second embodiment, the polymer further comprises a plurality of third repeat units represented by the following structural formula:

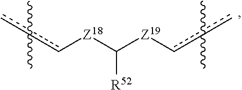

wherein:
Z¹⁸ and Z¹⁹ each independently is a C₁₋₃ alkylene or a bond; and
R⁵² is H, a C₁₋₁₂ alkyl, or a C₆₋₁₂ aryl. The remainder of features and example features of the fifty-eighth aspect is as described above with respect to the first through fifty-seventh aspects of the second embodiment.

In a fifty-ninth aspect of the second embodiment, R⁵² is H or a C₁₋₃ alkyl. For example, R⁵² is H. Alternatively, R⁵³ is methyl. The remainder of features and example features of the fifty-ninth aspect is as described above with respect to the first through fifty-eighth aspects of the second embodiment.

In a sixtieth aspect of the second embodiment, the plurality of first repeat units comprises a repeat unit represented by the following structural formula:

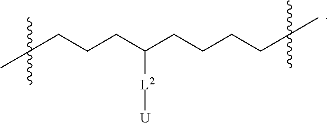

The remainder of features and example features of the sixtieth aspect is as described above with respect to the first through fifty-ninth aspects of the second embodiment.

In a sixty-first aspect of the second embodiment, the plurality of first repeat units comprises a repeat unit represented by the following structural formula:

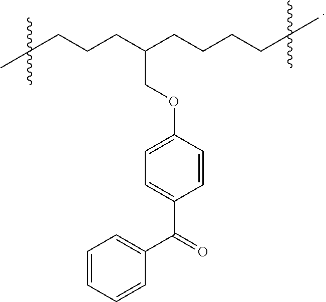

The remainder of features and example features of the sixty-first aspect is as described above with respect to the first through sixtieth aspects of the second embodiment.

In a sixty-second aspect of the second embodiment, the plurality of second repeat units comprises a repeat unit represented by the following structural formula

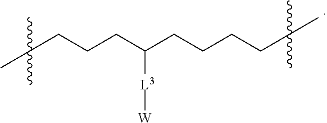

The remainder of features and example features of the sixty-second aspect is as described above with respect to the first through sixty-first aspects of the second embodiment.

In a sixty-third aspect of the second embodiment, the plurality of second repeat units comprises a repeat unit represented by the following structural formula:

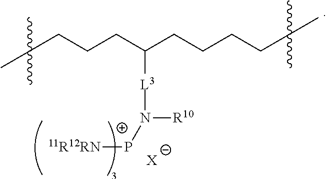

For example, the plurality of second repeat units comprises a repeat unit represented by the following structural formula:

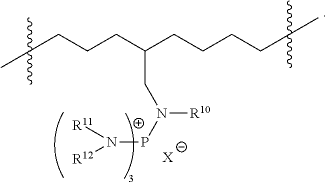

The remainder of features and example features of the sixty-third aspect is as described above with respect to the first through sixty-second aspects of the second embodiment.

In a sixty-fourth aspect of the second embodiment, the plurality of second repeat units comprises a repeat unit represented by any one of the following structural formulas:

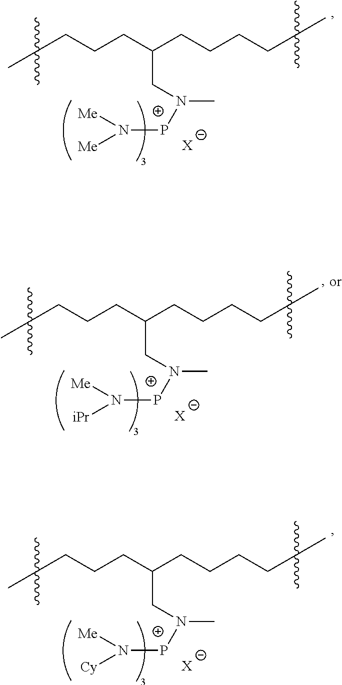

wherein Me is methyl, iPr is isopropyl and Cy is cyclohexyl. The remainder of features and example features of the sixty-fourth aspect is as described above with respect to the first through sixty-third aspects of the second embodiment.

In a sixty-fifth aspect of the second embodiment, the plurality of first repeat units comprises a repeat unit represented by the following structural formula:

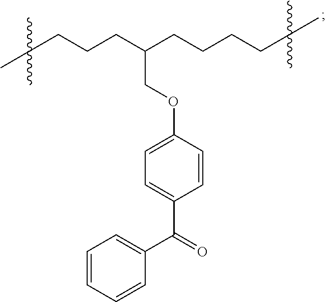

the plurality of second repeat units comprises a repeat unit represented by one of the following structural formulas:

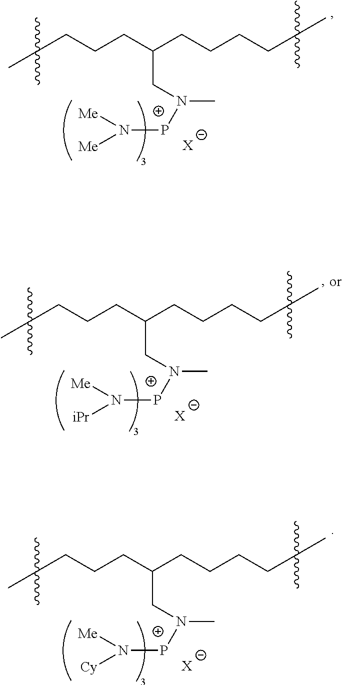

The remainder of features and example features of the sixty-fifth aspect is as described above with respect to the first through sixty-fourth aspects of the second embodiment.

In a sixty-sixth aspect of the second embodiment, the polymer comprises from about 0.5 mol-% to about 50 mol-% of the first repeat units. For example, the polymer comprises from about 2 mol-% to about 20 mol-% of the first repeat units, such as about 2 mol-% or about 5 mol-%. The remainder of features and example features of the sixty-sixth aspect is as described above with respect to the first through sixty-fifth aspects of the second embodiment.

In a sixty-seventh aspect of the second embodiment, the polymer comprises from about 10 mol-% to about 80 mol-% of the second repeat units. For example, the polymer comprises from about 20 mol-% to about 60 mol-% of the second repeat units, such as about 28 mol-%, about 46 mol-%, or about 70 mol-%. The remainder of features and example features of the sixty-seventh aspect is as described above with respect to the first through sixty-sixth aspects of the second embodiment.

In a sixty-eighth aspect of the second embodiment, the number average molecular weight (MWn) of the polymer is from about 30,000 g/mol to about 500,000 g/mol. For example, the MWn of the polymer is from about 50,000 g/mol to about 360,000 g/mol. The remainder of features and example features of the sixty-eighth aspect is as described above with respect to the first through sixty-seventh aspects of the second embodiment. The remainder of features and example features of the sixty-eighth aspect is as described above with respect to the first through sixty-seventh aspects of the second embodiment.

In a sixty-ninth aspect of the second embodiment, the polymer is cross-linked. The remainder of features and example features of the sixty-ninth aspect is as described above with respect to the first through sixty-eighth aspects of the second embodiment.

In a third embodiment the invention is a cross-linked polymer, comprising: a plurality of first repeat units selected from cross-linking moieties represented by structural formula (IIa) or structural formula (IIb):

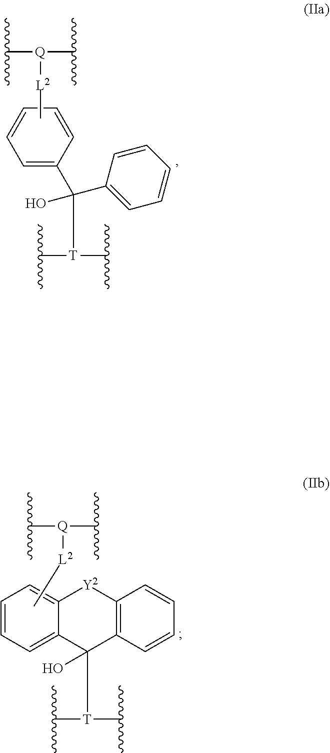

a plurality of second repeat units represented by structural formula (III):

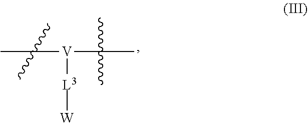

wherein:
[symbol] is a point of attachment to adjacent repeat units of the polymer;
Q is a moiety represented by one of the following structural formulas:

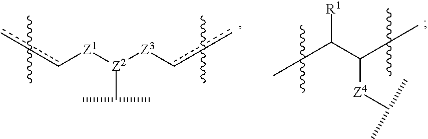

V is a moiety represented by one of the following structural formulas:

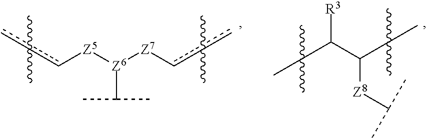

W is a C₁₋₁₂ alkyl or a moiety represented by one of the structural formulas selected from:

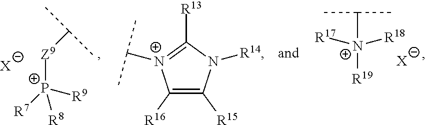

T, for each occurrence independently, is a C₂₋₈ alkylene,
[symbol] is a point of attachment to adjacent repeat units of the polymer;
[symbol] is a point of attachment to L²;
[symbol] is a point of attachment to L³
   and further wherein:
[symbol] is a double bond or a single bond;
Z¹, Z³, Z⁵, and Z⁷ each independently is a C₁₋₃ alkylene or a bond;
Z² is selected from —CHR⁵—, a C₅₋₁₂ cycloalkylene, and a 5 to 16-membered heterocyclylene;
Z⁴ is selected from a bond, a C₆₋₁₂ arylene, and CR², in which case R¹ and R² together with the carbon atoms to which they are attached form a C₅₋₁₂ cycloalkyl or a 5 to 12-membered heterocyclyl;
Z⁶ is selected from —CHR⁶—, a C₅₋₁₂ cycloalkylene, and a 5 to 16-membered heterocyclylene;
Z⁸ is selected from a bond, a C₆₋₁₂ arylene, and CR⁴, in which case R³ and R⁴ together with the carbon atoms to which they are attached form a C₅₋₁₂ cycloalkyl or a 5 to 12 membered heterocyclyl;
Z⁹ is NR¹⁰ or a bond;
R¹, if present, and R³, if present, each independently is selected from H, a C₁₋₁₂ alkyl, and C₆₋₁₂ aryl;
R⁵ and R⁶ each independently is H or a C₁₋₁₂ alkyl;
R⁷, R⁸, and R⁹ each independently is selected from NR¹¹R¹², a C₆₋₁₂ aryl, and 5 to 12-membered heterocyclyl;
R¹⁰ is a C₁₋₁₂ alkyl;
R¹¹ and R¹² each independently is C₁₋₁₂ alkyl or a C₃₋₁₂ cycloalkyl, or R¹¹ and R¹² together with the nitrogen atom to which they are attached form a 5 to 12-membered heterocyclyl;
R¹³ is selected from a C₁₋₁₂ alkyl, C₆₋₁₂ aryl, and 5 to 12-membered heterocyclyl;
R¹⁴ is a C₁₋₁₂ alkyl or a C₃₋₁₂ cycloalkyl;
R¹⁵ and R¹⁶ each independently is selected from C₁₋₁₂ alkyl, C₆₋₁₂ aryl, and 5 to 12-membered heterocyclyl; or R¹⁵ and R¹⁶ together with the carbon atoms to which they are attached form a C₆₋₁₂ aryl or a 5 to 12-membered heterocyclyl; and
R¹⁷, R¹⁸, and R¹⁹ each independently is a C₁₋₁₂ alkyl or a C₃₋₁₂ cycloalkyl; or R¹⁸ and R¹⁹ together with the nitrogen atom to which they are attached form a 5 to 12-membered heterocyclyl and (i) R¹⁷ is a C₁₋₁₂ alkyl or a C₃₋₁₂ cycloalkyl, or (ii) R¹⁷ and at least one atom of L³, if present, together with the nitrogen atom to which L³ and R¹⁷ are attached form a 5- to 12-membered heterocyclyl; or
R¹⁷, R¹⁸, and R¹⁹ together with the nitrogen atom to which they are attached form a bicyclic 5 to 12-membered heterocyclyl; and
L² is selected from a C₁₋₁₂ alkylene, C₆₋₁₂ arylene, C₆₋₁₂ arylene-C₁₋₁₂ alkylene, C₁₋₁₂ alkylene-C₆₋₁₂ arylene, C₁₋₁₂ alkylene-O—C₁₋₁₂ alkylene, (O—C₁₋₁₂ alkylene)ₘ, (C₁₋₁₂ alkylene-O)ₘ, C₁₋₁₂ alkylene-NH—C₁₋₁₂ alkylene, C₁₋₁₂ alkylene-N(C₁₋₁₂ alkyl)-C₁₋₁₂ alkylene, (NH—C₁₋₁₂ alkylene)ₘ, and (C₁₋₁₂ alkylene-NH)ₘ;
L³ is selected from a C₁₋₁₂ alkylene, C₆₋₁₂ arylene, C₆₋₁₂ arylene-C₁₋₁₂ alkylene, C₁₋₁₂ alkylene-C₆₋₁₂ arylene, C₁₋₁₂ alkylene-O—C₁₋₁₂ alkylene, (O—C₁₋₁₂ alkylene) n, (C₁₋₁₂ alkylene-O) n, C₁₋₁₂ alkylene-NH—C₁₋₁₂ alkylene, C₁₋₁₂ alkylene-N(C₁₋₁₂ alkyl)-C₁₋₁₂ alkylene, (NH—C₁₋₁₂ alkylene) n, (C₁₋₁₂ alkylene-NH) n, and a bond;
m is an integer between 1 and 6;
n is an integer between 1 and 6;
Y² is selected from —C(═O)—, O, S, NH, N(C₁₋₁₂ alkyl), and a bond;
X⁻ is selected from F⁻, Cl⁻, Br⁻, OH⁻, NO⁻₂, CN⁻, HCO₃⁻, CO₃²⁻, PF₆⁻, BF₄⁻, a C₁₋₁₂ carboxylate and a C₁₋₁₂ alkoxide;
provided that W is C₁₋₁₂ alkyl only when V is a cationic moiety, and wherein:
each C₁₋₁₂ alkylene, C₆₋₁₂ arylene, C₅₋₁₂ cycloalkylene, 5 to 16-membered heterocyclylene, C₁₋₁₂ alkyl, C₃₋₁₂ cycloalkyl, C₅₋₁₂ cycloalkyl, C₆₋₁₂ aryl, and 5 to 12-membered heterocyclyl is independently optionally substituted with 1 to 6 substituents independently selected from the group consisting of F, Cl, Br, OH, NH₂, oxo, a C₁₋₁₂ alkyl, C₆₋₁₂ aryl, C₁₋₁₂ haloalkyl, C₁₋₁₂ alkoxy, C₆₋₁₂ aryl, C₆₋₁₂ aryloxy, NH(C₁₋₁₂ alkyl), N(C₁₋₁₂ alkyl)₂, C(O)O(C₁₋₁₂ alkyl), and C(O)NH(C₁₋₁₂ alkyl).

In a first aspect of the third embodiment, the plurality of first repeat units comprises a cross-linking moiety represented by the following structural formula:

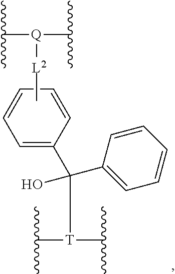

Q is a moiety represented by the following structural formula:

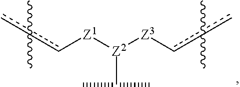

is a moiety represented by the following structural formula:

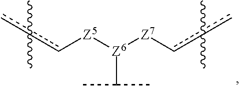

and W is a C₁₋₁₂ alkyl or a moiety represented by one of the structural formulas selected from:

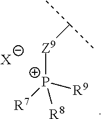

In a second aspect of the third embodiment, the plurality of first repeat units comprises a cross-linking moiety represented by the following structural formula:

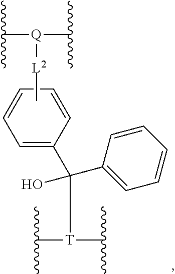

Q is a moiety represented by the following structural formula:

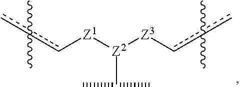

V is a moiety represented by the following structural formula:

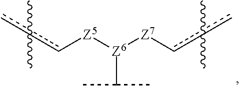

and W is a C₁₋₁₂ alkyl or a moiety represented by one of the structural formulas selected from:

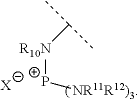

The remainder of features and example features of the second aspect is as described above with respect to the first aspect of the third embodiment.

In a third aspect of the third embodiment, the plurality of first repeat units comprises a cross-linking moiety represented by the following structural formula:

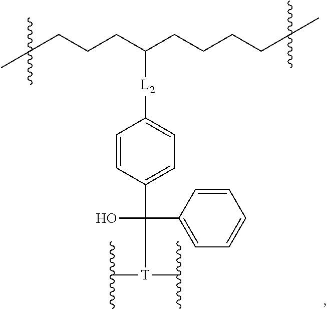

and the plurality of second repeat units comprises a repeat unit represented by the following structural formula:

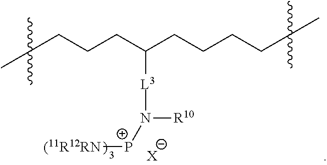

The remainder of features and example features of the third aspect is as described above with respect to the first through second aspects of the third embodiment.

In a fourth aspect of the third embodiment, the plurality of first repeat units comprises a cross-linking moiety represented by the following structural formula:

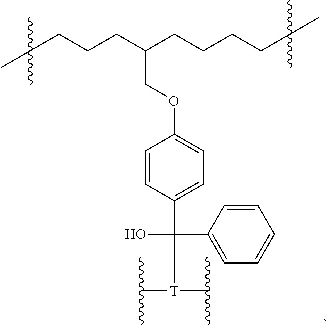

and the plurality of second repeat units comprises a repeat unit represented by one of the following structural formulas:

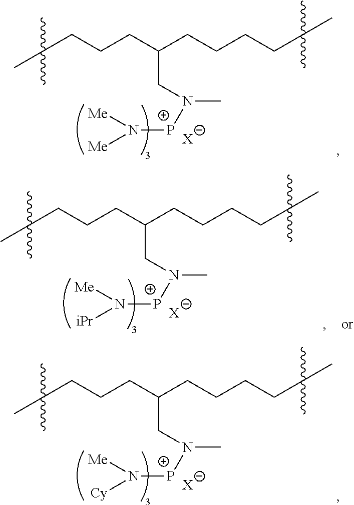

wherein Me is methyl, iPr is isopropyl, and Cy is cyclohexyl. The remainder of features and example features of the fourth aspect is as described above with respect to the first through third aspects of the third embodiment.

In a fifth aspect of the third embodiment, Q, V, W, L², and L³ are as described in any of the twelfth through fifty-seventh aspects of the second embodiment. The remainder of features and example features of the fifth aspect is as described above with respect to the first through fourth aspects of the third embodiment.

In a sixth aspect of the third embodiment, the cross-linked polymer further comprises a plurality of third repeat units represented by the following structural formula:

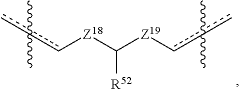

wherein: Z¹⁸ and Z¹⁹ each independently is a C₁₋₃ alkylene or a bond; and R⁵² is H, a C₁₋₁₂ alkyl, or a C₆₋₁₂ aryl. The remainder of features and example features of sixth aspect is as described above with respect to the first through fifth aspects of the third embodiment.

In a fourth embodiment the invention is a composite material, comprising a reinforcement material and a polymer described herein with respect to the second embodiment and various aspects thereof, or a cross-linked polymer described herein with respect to the third embodiment and various aspects thereof.

In a first aspect of the fourth embodiment, the reinforcement material is a porous material; and the porous material is impregnated with the polymer or the cross-linked polymer.

In a fifth embodiment the invention is a membrane, comprising a film of the polymer described herein with respect to the second embodiment and various aspects thereof, the cross-linked polymer described herein with respect to the third embodiment and various aspects thereof; or the composite material described herein with respect to the fourth embodiment and various aspects thereof.

In a sixth embodiment the invention is a membrane electrode assembly, comprising a membrane described herein with respect to the fifth embodiment and various aspects thereof and an electrode.

In a seventh embodiment the invention is an electrochemical device, comprising a membrane electrode assembly described herein with respect to the sixth embodiment and various aspects thereof and a current collector.

In a first aspect of the seventh embodiment, the device is an electrolyzer.

### EXEMPLIFICATION

The examples below describe methods of synthesis of the monomers, polymers, and AEIs of the present disclosure. The examples also provide methods of manufacturing and characterization of the AEMs and rAEMs of the disclosure.

### Abbreviations

COE cyclooctene
TEA triethylamine
THF tetrahydrofuran
ACN acetonitrile
Grubbs' Gen II catalyst (1,3-Bis(2,4,6-trimethylphenyl)-2-imidazolidinylidene)dichloro(phenylmethylene)(tricyclohexylp hosphine)ruthenium
Crabtree's catalyst (1,5-Cyclooctadiene)(pyridine)(tricyclohexylphosphine)-Ir(I) PF₆

### Example 1. Synthesis of COE-Benzophenone Monomer

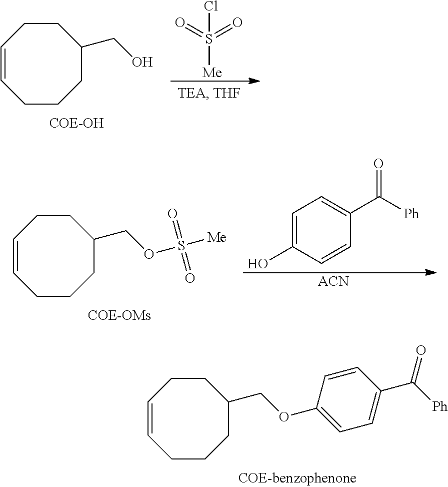

COE-OH (10 g, 71 mmol) was combined with triethylamine (8.1 g, 80 mmol) and tetrahydrofuran (anhydrous, 100 mL) in a 250 mL round bottom flask. The solution was cooled to 0° C. in an ice bath and methane sulfonyl chloride was added dropwise under the flow of nitrogen. The reaction was warmed to room temperature and allowed to stir for 24 h. The resulting heterogeneous mixture was filtered to remove solids and the organic layer was concentrated, in vacuo, to remove tetrahydrofuran. The crude oil was dissolved in diethyl ether and washed with KOH (1M), water, and brine. The organic layer was dried over magnesium sulfate and concentrated, in vacuo, to produce COE-OMs as a light yellow oil (13.3 g).

COE-OMs (5 g, 23 mmol) was combined in a 250 mL round bottom flask with potassium carbonate (7 g, 50 mmol), acetonitrile (50 mL) and 4-hydroxybenzophenone (4.8 g, 24 mmol). The round bottom flask was fitted with a reflux condenser and the reaction was heated to reflux for 24 h. The reaction was cooled, filtered, and concentrated, in vacuo, to remove acetonitrile. The resulting crude oil was dissolved in ethyl acetate and washed with 1M KOH, water, and brine. The organic layer was dried over magnesium sulfate, filtered, and concentrated, in vacuo, to produce COE-Benzophenone as an off-white, waxy solid (4.1 g).

### Example 2. Synthesis of Isopropyl-Methyl Tetrakis® Monomer

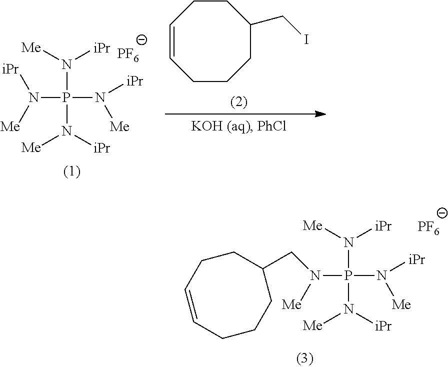

Tris(isopropyl(methyl)amino)(methylamino)phosphonium hexafluorophosphate (1) was synthesized as detailed in Treichel, M. et al. Macromolecules, 2020, 53, 8509.

Compound (1) (5.0 g, 12 mmol) was combined with chlorobenzene (12 mL) in a 250 mL round bottom flask. A potassium hydroxide solution (50 wt %, 10.0 g, 178 mmol) was charged followed by COE-iodide (2) (4.4 g, 18 mmol). The reaction was heated to 60° C. for 24 h. The reaction was cooled and layers separated. The aqueous layer was extracted with dichloromethane and all organic layers were washed with 1M HCl, saturated potassium hexafluorophosphate, and water. The organic layer was dried over magnesium chloride, concentrated, in vacuo, to produce a crude oil. The oil was dissolved in a minimal amount of dichloromethane and precipitated into diethyl ether to produce Tetrakis® monomer (3) as an off-white, waxy solid (3.9 g).

### Example 3. Synthesis of a Non-Cross-Linked Benzophenone-Containing Polyelectrolyte

Tetrakis® Monomer (0.62 g, 1.1 mmol), COE-Benzophenone (0.10 g, 0.32 mmol), and cyclooctene (0.28 g, 2.5 mmol) were dissolved in dichloromethane while under inert atmosphere. Grubbs' Gen II catalyst (7 mg, 0.01 mmol) was added to the solution and the reaction was stirred for 18 h. The resulting polymer was dissolved in a 2:1 (v:v) dichloromethane:methanol mixture and added to a pressure vessel. Crabtree's catalyst (6.4 mg, 0.01 mmol) was added, and the reaction was pressurized to 800 psi of hydrogen and heated to 55° C. for 17 h. The reaction was cooled to room temperature and the solvent removed to produce 0.96 g of a Tetrakis-BXL, non-cross-linked polymer.

### Example 4. Cross-Linking of a Benzophenone-Containing Polyelectrolyte

AEIs comprising Tetrakis® cations with cyclohexyl, methyl substitution pattern were used in the experiments described below.

### I. Preparation of Free-Standing Cross-Linked Polyelectrolyte Anionic Exchange Membranes (AEMs).

### A. Drop-Casting.

A solution was made by dissolving 520 mg of the polyelectrolyte in 15 mL of a solvent system chloroform:methanol (4:1). While the polyelectrolyte was dissolving, a large casting dish was leveled, using a micrometer, on the countertop at ambient temperature. When the polyelectrolyte was fully dissolved, it was filtered through a syringe and glass wool to remove any large particulates. The solution was then poured into the large casting dish with a bell jar placed on top to create a dust free environment. After staying in the dish overnight the membrane was cross-linked in the dish by UV light for 1 hour then lifted from the dish with water and air dried. The UV-crosslinking procedure was performed as follows: the membrane was placed flat under the UV light 2 inches from the UV bulb (100 W, 365 nm) in the center of the membrane to make sure there is adequate light coverage over the membrane. A cover was placed over the light source and the membrane. The membrane was irradiated with UV light for 1 hour.

### B. Tape-Casting.

An 8.5 wt % solution of the polyelectrolyte T-28-120-BXL5 in a 3:1 water:n-propanol solvent system was prepared by heating the mixture up to 62° C. for 1 hour while stirring the mixture at a low rpm, then heating the mixture at 90° C. for 2 hours while stirring at a higher rpm until all the polymer is dissolved. Once the polymer was fully dissolved, the solution was cooled to ambient temperature and filtered through glass wool in a syringe. The solution was then poured over a pre-cleaned glass sheet and a pre-cleaned draw bar was activated to draw over the polymer solution creating a film in the targeted thickness. The glass sheet with the membrane was then put into the 80° C. oven for 3 hours and then moved to a 120° C. vacuum oven overnight. The membrane is lifted by addition of water, dried at ambient temperature, and finally cross-linked for an hour as described in part A.

II. Preparation of Reinforced Cross-Linked Polyelectrolyte Anionic Exchange Membranes (rAEMs).

A 4 wt % solution of the polyelectrolyte was prepared by dissolving the polyelectrolyte in a 3:1 water:n-propanol solvent system. The solution was gently heated until all polymer was dissolved, and the solution was cooled down to ambient temperature and filtered through glass wool in a syringe. A hot plate coated with a clean sheet of Teflon film was heated to 80° C. The polymer support (PP or PE) was cleaned for 1 hour in a 25° C. in a sonic bath in pure ethanol, then was allowed to air dry before the before weighing. The support was then put into a spray-coating frame and clamped down. The polymer solution was added to the spray gun and applied to both sides of the support at 30 psi. The impregnated support was then set to dry on the hot plate for 5 minutes, or was placed for 2 minutes in an oven preheated to 80° C. Mass and thickness of the impregnated support were measured before another coat of the polyelectrolyte was applied. When the final coat was dry, the impregnated support was placed under UV light for 1 h to achieve polymer crosslinking according to the procedure described in section IA above.

### Example 5. Characterization Methods of the Cross-Linked Membranes

The methods described below were used with free-standing AEMs as well as rAEMs.

### A. In-Plane Hydroxide Conductivity at Ambient & Variable Temperature

High performance AEMs exhibit relatively low ionic resistance and high ionic conductivity, as conductivity is inversely proportional to resistance. Calculating ionic conductivity from a measured resistance simplifies the comparison of AEMs by normalizing to a specified area. The Bekktech cell (Scribner Associates) was used to measure in-plane hydroxide conductivity using Electrochemical Impedance Spectroscopy (EIS). The measurements were performed in an environmental chamber, which was purged with inert gas, to reduce the complications of carbonate formation during the experiment. The hydroxide conductivity in liquid water at ambient/room temperature and 80° C. was obtained.

### B. In-Plane Hydroxide Conductivity at Variable Temperature & Humidity

Often conductivity is measured by submerging the samples in liquid water, which simplifies the equipment setup, making the results more reproducible. However, AEMs in fuel cells and electrolyzers are exposed to drier conditions, with relative humidity (RH) as low as 50%. Measurements in liquid water do not accurately predict the conductivity decrease observed under these dehydrated conditions. Measuring hydroxide conductivity at 70% RH and 80° C. was used to obtain an indication on how AEMs will perform under operating humidity. After modifying the equipment setup by adding a humidification chamber to the CO₂-free environmental chamber, the electrochemical experiment was conducted with similar parameters to ambient measurements.

### C. Through-Plane Hydroxide Conductivity at Variable Temperature

High performance AEMs exhibit relatively low ionic resistance and high ionic conductivity. Low resistance and high conductivity in the through-plane direction is an indication that the AEM will have good ion mobility in an operating device. Through-plane conductivity measurements have been historically difficult to obtain and reproduce due to the lack of standardized hardware. Through-plane values are often estimated from in-plane conductivity; however, the anisotropic properties AEMs limits the accuracy of this method. A through-plane conductivity cell, with dimensions and design features similar to the Bekktech cell, was used. Conductivity was calculated from EIS, similar to an in-plane measurement, using a model circuit.

AEM samples were prepared with the appropriate dimensions, a 1 cm×1 cm square is required for Ecolectro's cell. The AEM samples were exchanged for hydroxide using a technique appropriate for the polymer type. Immediately following exchange, the AEMs were quickly mounted into the cell, the cell was placed in liquid water equilibrated to the desired temperature and the conductivity was measured. Measurements were completed in an environmental chamber that was purged with inert gas to limit carbonate contamination.

### D. Area Specific Resistance (ASR)

High performance AEMs exhibit relatively low ionic resistance and high ionic conductivity. Low resistance in the through-plane direction is an indication that the AEM will have good ion mobility in an operating device. This specific measurement is known as Areal Specific Resistance (ASR). A device to measure through-plane conductivity was used to directly measure EIS and the ASR in this configuration. ASR was calculated from through-plane resistance.

### E. Mechanical Properties

AEMs ideally have high mechanical strength when hydrated at high temperatures and sufficient handling properties to be incorporated into a membrane electrode assembly (MEA). Reporting the stress and elongation at break is a universal method for characterizing intrinsic polymer mechanical properties.

The mechanical properties of the AEMs were measured at 50° C., 50% RH with a tensile tester. The AEM samples were pre-equilibrated for 48 hours prior to testing the mechanical properties in an environmental chamber, both at 50° C., 50% RH. Most AEMs were analyzed in the native halide form.

### F. Alkaline Stability

Alkaline stability studies were used to evaluate the chemical stability of AEMs in conditions that are relevant to operating alkaline electrolyzers or fuel cells. The disclosed AEMs and the commercial AEMs were cut into appropriate dimensions for the various analytical techniques.

The selected condition was aqueous 1M KOH at 80° C. The AEMs were exchanged into the hydroxide form using a procedure appropriate for each AEM. The samples were immediately submerged in 1M KOH and stored at 80° C. for 200-2,000 hours. After 200-2,000 hours, the samples were removed and prepared for analysis according to the protocols for the specific analytical technique.

Three analytical techniques were used to evaluate the AEMs. The in-plane hydroxide conductivity at room temperature, IEC or in-plane chloride conductivity at 80° C.

### G. Water Uptake and Swelling

Hydration of AEMs is critical for mobility of hydroxide ions through the MEA and be an efficient reactant. However, excessive swelling can negatively impact the MEA structure by causing large dimensional changes during operation. To understand the forces that an AEM experiences under operating conditions, the swelling (i.e. change in dimension; where X=length; Y=width; Z=thickness) and water uptake (i.e. the mass of water that is absorbed) were determined.

The dimensions and weight of an AEM samples in the dry halide form were measured. The AEMs were converted to the hydroxide form using a procedure appropriate for the material and then stored in pure water at ambient/room temperature or 80° C. The AEMs in the hydroxide form were measured again after treatment and the changes in dimension and weight were reported relative to the dry halide form.

### H. Ion Exchange Capacity (IEC)

The ability to transport ions is a fundamental and distinct property of polymer electrolytes. The cation content in the polymer impacts anion mobility, in addition to swelling and water uptake. The cation content is typically controlled by the synthetic route and a theoretical value for Ion Exchange Capacity (IEC) can be determined from synthesis inputs. The theoretical IEC is defined as the mmol or meq of ion per gram of polymer. Hydroxide is typically the ion concentration that is measured, which translates to the number of cations in the polymer. The measured IEC is lower than the theoretical when some ionic sites in the polymer sample are blocked and not accessible. The theoretical and measured IEC values will match when the polymer identity is as expected and all cationic groups in the polymer are involved in ion transport.

The weights of dry AEM samples were recorded. The AEM samples were converted to the hydroxide form using a method appropriate for the polymer type. The samples were then soaked in HCl, and the hydroxide in the polymer reacted with some of the acid. The amount of HCl that reacted with the AEM/AEI was determined by titration of the HCl solution, which provided the effective hydroxide content and accessible cations in the polymer. The mmol of hydroxide determined from the titration was divided by the weight of the polymer sample to obtain the IEC.

H. Polymer Loading and Volume Determination for rAEM Fabrication
1) Cut a defined piece of support, measure X (length, initial) and Y (width, initial).
2) Cleaned, dry, and weigh support—M(initial)
3) Fabricated rAEM with polymer
4) Measured X(final), Y(final), M(final) and Z(Thickness)

Polymer loading and volume were determined according top the following equations:

$$Polymer Loading ( by mass ) = \frac{[ M ( final ) - M ( initial ) ]}{X ( final ) * Y ( final )}$$

$$Polymer Volume = \frac{[ M ( final ) - M ( initial ) ]}{X ( final ) * Y ( final ) * Z}$$

While this invention has been particularly shown and described with references to example embodiments thereof, it will be understood by those skilled in the art that various changes in form and details may be made therein without departing from the scope of the invention encompassed by the appended claims.

The teachings of all patents, published applications and references cited herein are incorporated by reference in their entirety.

1. A compound represented by structural formula (I): 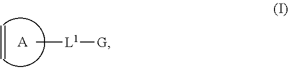
wherein:
the moiety represented by 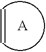
is a moiety represented by any one of the following structural formulas: 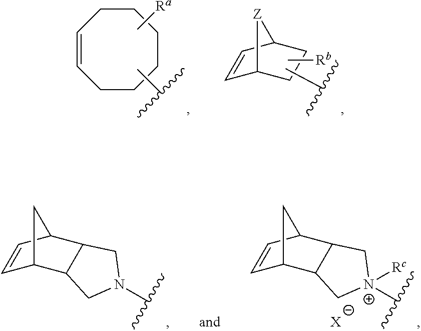
wherein:
R^{a}, R^{b}, and R^{c} each independently is H or a C₁₋₁₂ alkyl;
Z is selected from CH₂, O, NH, and N(C₁₋₁₂ alkyl); and
X⁻ is selected from F⁻, Cl⁻, Br⁻, OH⁻, NO⁻₂, CN⁻, HCO₃⁻, CO₃²⁻, PF₆⁻, BF₄⁻, a C₁₋₁₂ carboxylate, and a C₁₋₁₂ alkoxide,
L¹ is selected from a (O—C₁₋₁₂ alkylene)ₖ, (C₁₋₁₂ alkylene-O)ₖ, C₁₋₁₂ alkylene, C₆₋₁₂ arylene, C₆₋₁₂ arylene-C₁₋₁₂ alkylene, C₁₋₁₂ alkylene-C₆₋₁₂ arylene, C₁₋₁₂ alkylene-O—C₁₋₁₂ alkylene, C₁₋₁₂ alkylene-NH—C₁₋₁₂ alkylene, C₁₋₁₂ alkylene-N(C₁₋₁₂ alkyl)-C₁₋₁₂ alkylene, (NH—C₁₋₁₂ alkylene)ₖ, (C₁₋₁₂ alkylene-NH)ₖ;
the moiety represented by G is selected from a moiety represented by any one of the following structural formulas: 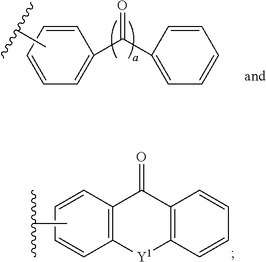 [symbol]
is a point of attachment of the moiety represented by G to L¹;
k is an integer between 1 and 6;
a is 1 or 2;
Y¹ is —C(═O)—, O, S, NH, N(C₁₋₁₂ alkyl), or a bond; and
and further wherein:
each C₁₋₁₂ alkylene, C₆₋₁₂ arylene, C₁₋₁₂ alkyl, C₇₋₈ cycloalkenyl, and 7 to 12-membered heterocycloalkenyl is independently optionally substituted with 1 to 6 substituents selected from the group consisting of F, Cl, Br, OH, NH₂, C₁₋₁₂ alkyl, C₆₋₁₂ aryl a C₁₋₁₂haloalkyl, C₁₋₁₂ alkoxy, C₆₋₁₂ aryl, C₆₋₁₂ aryloxy, NH(C₁₋₁₂ alkyl), N(C₁₋₁₂ alkyl)₂, C(O)O(C₁₋₁₂ alkyl), and C(O)NH(C₁₋₁₂ alkyl).

2. The compound of claim 1, wherein
the moiety represented by G is a moiety represented by the following structural formula: 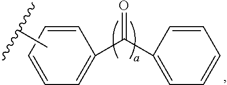
and
L¹ is C₁₋₁₂ alkylene, (O—C₁₋₁₂ alkylene)ₖ or (C₁₋₁₂ alkylene-O)ₖ.

3-4. (canceled)

5. The compound of claim 1, wherein
the moiety represented by G is a moiety represented by the following structural formula: 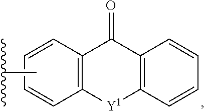
and
L¹ is C₁₋₁₂ alkylene, (O—C₁₋₁₂ alkylene)ₖ or (C₁₋₁₂ alkylene-O)ₖ.

6-10. (canceled)

11. The compound of claim 1, wherein the moiety represented by 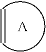
is

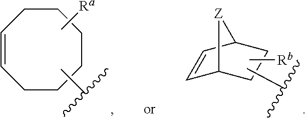

12-19. (canceled)

20. The compound of claim 1, wherein the moiety represented by 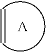
is

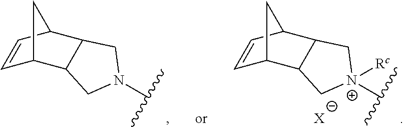

21-22. (canceled)

23. The compound of claim 1, wherein L¹ is (O—C₁₋₁₂ alkylene)ₖ or (C₁₋₁₂ alkylene-O)ₖ.

24-26. (canceled)

27. The compound of claim 1, wherein the compound is selected from:

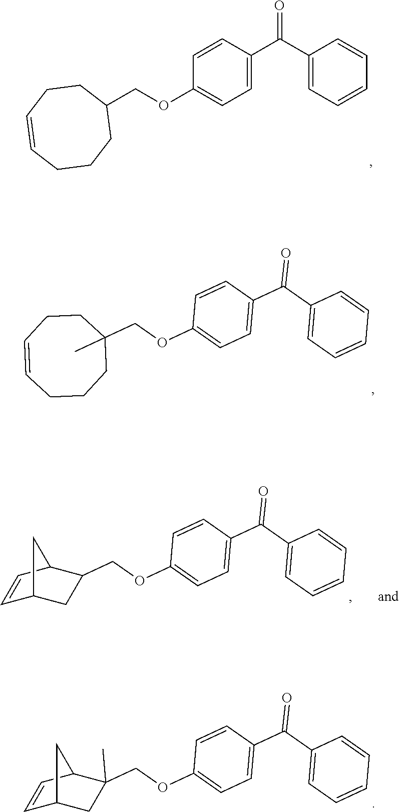

28. A polymer, comprising:
a plurality of first repeat units represented by structural formula (II): 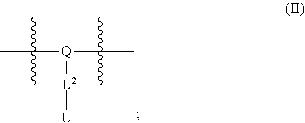
and
a plurality of second repeat units represented by structural formula (III): 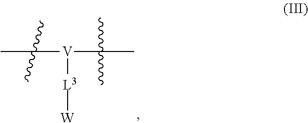
wherein:
Q is a moiety represented by one of the following structural formulas: 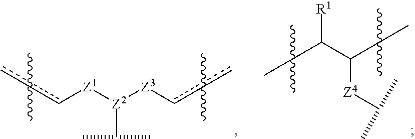
U is a moiety represented by one of the following structural formulas: 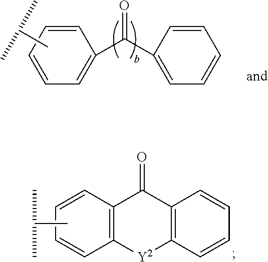
V is a moiety represented by one of the following structural formulas: 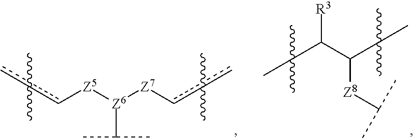
W is a C₁₋₁₂ alkyl or a moiety represented by one of the structural formulas selected from: 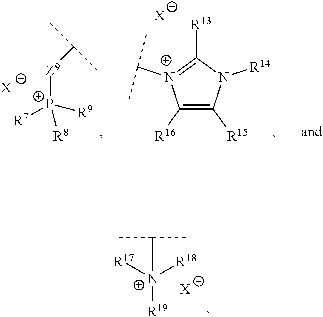 [symbol]
is a point of attachment to adjacent repeat units of the polymer; [symbol]
is a point of attachment to L²; [symbol]
is a point of attachment to L³
and further wherein: [symbol]
is a double bond or a single bond;
Z¹, Z³, Z⁵, and Z⁷ each independently is a C₁₋₃ alkylene or a bond;
Z² is selected from —CHR⁵—, a C₅₋₁₂ cycloalkylene, and a 5 to 16-membered heterocyclylene;
Z⁴ is selected from a bond, a C₆₋₁₂ arylene, and CR², wherein
if Z⁴ is CR², then R¹ and R² together with the carbon atoms to which they are attached form a C₅₋₁₂ cycloalkyl or a 5 to 12-membered heterocyclyl, and
if Z⁴ is selected from a bond and a C₆₋₁₂ arylene, then R¹ is selected from H, a C₁₋₁₂ alkyl, and C₆₋₁₂ aryl;
Z⁶ is selected from —CHR⁶—, a C₅₋₁₂ cycloalkylene, and a 5 to 16-membered heterocyclylene;
Z⁸ is selected from a bond, a C₆₋₁₂ arylene, and CR⁴, wherein
if Z⁸ is CR⁴, then R³ and R⁴ together with the carbon atoms to which they are attached form a C₅₋₁₂ cycloalkyl or a 5 to 12 membered heterocyclyl; and
if Z⁸ is selected from a bond and a C₆₋₁₂ arylene, then R³ is selected from H, a C₁₋₁₂ alkyl, and C₆₋₁₂ aryl;
Z⁹ is NR¹⁰ or a bond;
R⁵ and R⁶ each independently is H or a C₁₋₁₂ alkyl;
R⁷, R⁸, and R⁹ each independently is selected from NR¹¹R¹², a C₆₋₁₂ aryl, and 5 to 12-membered heterocyclyl;
R¹⁰ is a C₁₋₁₂ alkyl;
R¹¹ and R¹² each independently is C₁₋₁₂ alkyl or a C₃₋₁₂ cycloalkyl, or R¹¹ and R¹² together with the nitrogen atom to which they are attached form a 5 to 12-membered heterocyclyl;
R¹³ is selected from a C₁₋₁₂ alkyl, C₆₋₁₂ aryl, and 5 to 12-membered heterocyclyl;
R¹⁴ is a C₁₋₁₂ alkyl or a C₃₋₁₂ cycloalkyl;
R¹⁵ and R¹⁶ each independently is selected from C₁₋₁₂ alkyl, C₆₋₁₂ aryl, and 5 to 12-membered heterocyclyl; or R¹⁵ and R¹⁶ together with the carbon atoms to which they are attached form a C₆₋₁₂ aryl or a 5 to 12-membered heterocyclyl; and
R¹⁷, R¹⁸, and R¹⁹ each independently is a C₁-12 alkyl or a C₃₋₁₂ cycloalkyl; or
R¹⁸ and R¹⁹ together with the nitrogen atom to which they are attached form a 5 to 12-membered heterocyclyl and (i) R¹⁷ is a C₁₋₁₂ alkyl or a C₃₋₁₂ cycloalkyl, or (ii) R¹⁷ and at least one atom of L³, if present, together with the nitrogen atom to which L³ and R¹⁷ are attached form a 5- to 12-membered heterocyclyl; or
R¹⁷, R¹⁸, and R¹⁹ together with the nitrogen atom to which they are attached form a bicyclic 5 to 12-membered heterocyclyl; and
L² is selected from a C₁₋₁₂ alkylene, C₆₋₁₂ arylene, C₆₋₁₂ arylene-C₁₋₁₂ alkylene, C₁₋₁₂ alkylene-C₆₋₁₂ arylene, C₁₋₁₂ alkylene-O—C₁₋₁₂ alkylene, (O—C₁₋₁₂ alkylene)ₘ, (C₁₋₁₂ alkylene-O)ₘ, C₁₋₁₂ alkylene-NH—C₁₋₁₂ alkylene, C₁₋₁₂ alkylene-N(C₁₋₁₂ alkyl)-C₁₋₁₂ alkylene, (NH—C₁₋₁₂ alkylene)ₘ, and (C₁₋₁₂ alkylene-NH)ₘ;
L³ is selected from a C₁₋₁₂ alkylene, C₆₋₁₂ arylene, C₆₋₁₂ arylene-C₁₋₁₂ alkylene, C₁₋₁₂ alkylene-C₆₋₁₂ arylene, C₁₋₁₂ alkylene-O—C₁₋₁₂ alkylene, (O—C₁₋₁₂ alkylene)ₙ, (C₁₋₁₂ alkylene-O)ₙ, C₁₋₁₂ alkylene-NH—C₁₋₁₂ alkylene, C₁₋₁₂ alkylene-N(C₁₋₁₂ alkyl)-C₁₋₁₂ alkylene, (NH—C₁₋₁₂ alkylene)ₙ, (C₁₋₁₂ alkylene-NH)ₙ, and a bond;
m is an integer between 1 and 6;
n is an integer between 1 and 6;
b is 1 or 2;
Y² is selected from —C(═O)—, O, S, NH, N(C₁₋₁₂ alkyl), and a bond;
X⁻ is selected from F⁻, Cl⁻, Br⁻, OH⁻, NO⁻₂, CN⁻, HCO₃⁻, CO₃²⁻, PF₆⁻, BF₄⁻, a C₁₋₁₂ carboxylate and a C₁-12 alkoxide;
provided that W is C₁₋₁₂ alkyl only when V is a cationic moiety,
and wherein:
each C₁₋₁₂ alkylene, C₆₋₁₂ arylene, C₅₋₁₂ cycloalkylene, 5 to 16-membered heterocyclylene, C₁₋₁₂ alkyl, C₃₋₁₂ cycloalkyl, C₅₋₁₂ cycloalkyl, C₆₋₁₂ aryl, and 5 to 12-membered heterocyclyl is independently optionally substituted with 1 to 6 substituents independently selected from the group consisting of F, Cl, Br, OH, NH₂, oxo, C₁₋₁₂ alkyl, C₆₋₁₂ aryl, C₁₋₁₂ haloalkyl, C₁₋₁₂ alkoxy, C₆₋₁₂ aryl, C₆₋₁₂ aryloxy, NH(C₁₋₁₂ alkyl), N(C₁₋₁₂ alkyl)₂, C(O)O(C₁₋₁₂ alkyl), and C(O)NH(C₁₋₁₂ alkyl).

29. The polymer of claim 28, wherein:
U is a moiety represented by the following structural formula: 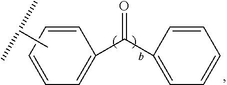
and
W is a moiety represented by the following structural formula:

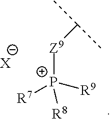

30. The polymer of claim 28, wherein U is a moiety represented by the following structural formula:

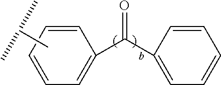

31. (canceled)

32. The polymer of claim 28, wherein U is a moiety represented by the following structural formula:

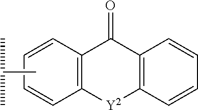

33-36. (canceled)

37. The polymer of claim 28, wherein:
Q is a moiety represented by the following structural formula: 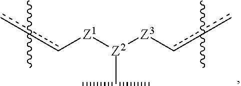
U is a moiety represented by the following structural formula: 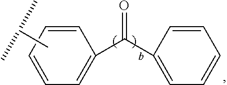
V is a moiety represented by the following structural formula: 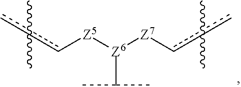
and
W is a moiety represented by the following structural formula:

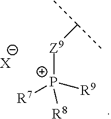

38-39. (canceled)

40. The polymer of claim 28, wherein Q is a moiety represented by any one of the following structural formulas: 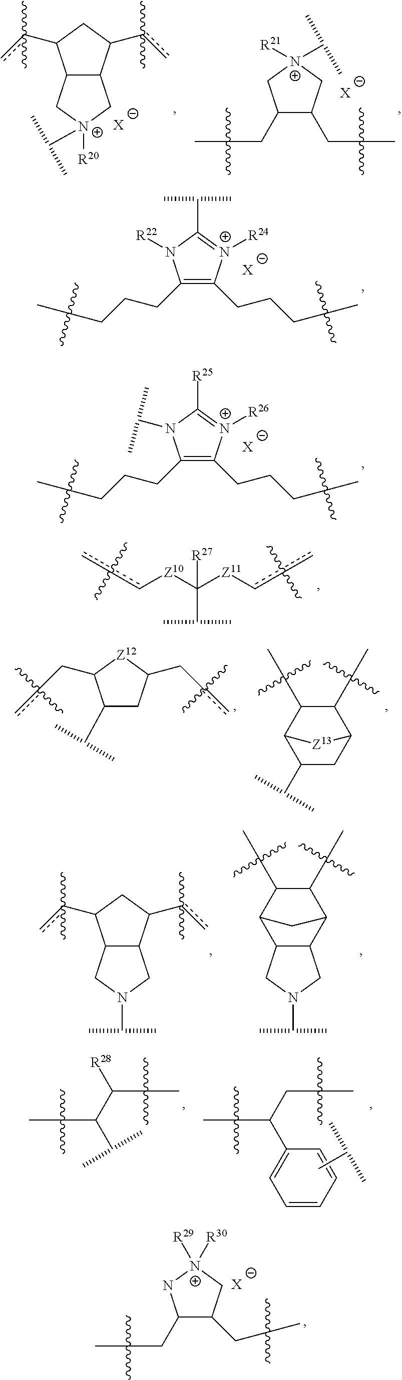 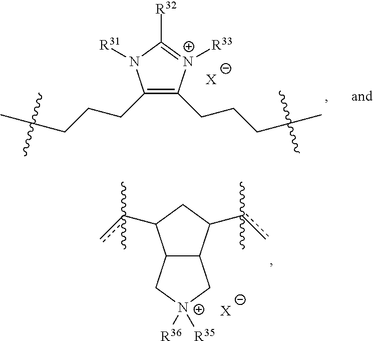
wherein:
R²⁰, R²¹, R²², R²⁴, R²⁶, R²⁹, R³⁰, R³¹, and R³³ each independently is a C₁₋₁₂ alkyl;
R²⁵ and R³² each independently is a C₆₋₁₂ aryl;
R²⁷ is H or a C₁₋₁₂ alkyl;
R²⁸ is H, a C₁₋₁₂ alkyl, or a C₆₋₁₂ aryl;
R³⁶ and R³⁵ each independently is a C₁₋₁₂ alkyl, or R³⁶ and R³⁵ together with the nitrogen atom to which they are attached form a 5- to 12-membered heterocyclyl;
Z¹⁰ and Z¹¹ each independently is a C₁₋₃ alkylene or a bond; and
Z¹² and Z¹³ each independently is selected from CH₂, O, NH, and N(C₁₋₁₂ alkyl).

41-57. (canceled)

58. The polymer of claim 28, wherein V is a moiety represented by any one of the following structural formulas: 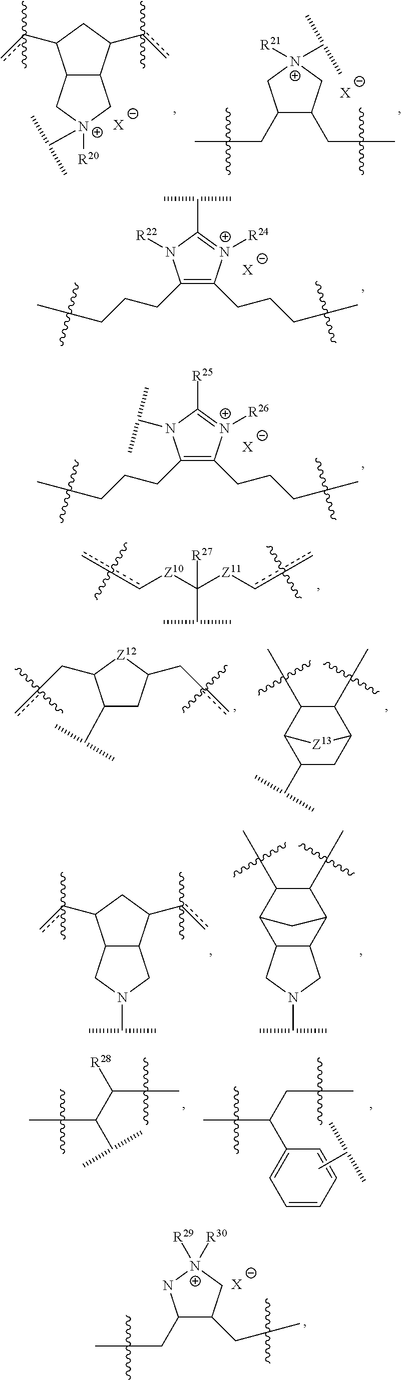 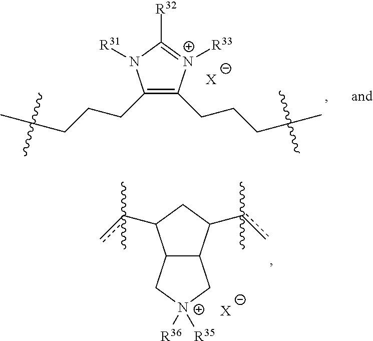
wherein:
R³⁷, R³⁸, R³⁹, R⁴⁰, R⁴², R⁴⁵, R⁴⁶, R⁴⁷, and R⁴⁹ each independently is a C₁₋₁₂ alkyl;
R⁴¹ and R⁴⁸ each independently is a C₆₋₁₂ aryl;
R⁴³ is H or a C₁₋₁₂ alkyl;
R⁴⁴ is H, a C₁₋₁₂ alkyl, or a C₆₋₁₂ aryl;
R⁵⁰ and R⁵ each independently is a C₁₋₁₂ alkyl, or R³⁶ and R³⁵ together with the nitrogen atom to which they are attached form a 5- to 12-membered heterocyclyl;
Z¹⁴ and Z¹⁵ each independently is a C₁₋₃ alkylene or a bond; and
Z¹⁶ and Z¹⁷ each independently is selected from CH₂, O, NH, and N(C₁₋₁₂ alkyl).

59-75. (canceled)

76. The polymer of claim 28, wherein W is

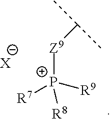

77-78. (canceled)

79. The polymer of claim 76, wherein:
Z⁹ is NR¹⁰;
R⁷, R⁸, and R⁹ each independently is NR¹¹R¹²; and
R¹¹ and R¹² each independently is a C₁₋₁₂ alkyl or a C₃₋₁₂ cycloalkyl.

80-92. (canceled)

93. The polymer of claim 28, wherein L² is (O—C₁₋₁₂ alkylene)ₘ or (C₁₋₁₂ alkylene-O)ₘ.

94-95. (canceled)

96. The polymer of claim 28, wherein L³ is a C₁₋₁₂ alkylene.

97-99. (canceled)

100. The polymer of claim 28, further comprising a plurality of third repeat units represented by the following structural formula: 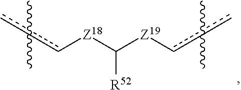
wherein:
Z¹⁸ and Z¹⁹ each independently is a C₁₋₃ alkylene or a bond; and
R⁵² is H, a C₁₋₁₂ alkyl, or a C₆₋₁₂ aryl.

101. (canceled)

102. The polymer of claim 28, wherein the plurality of first repeat units comprises a repeat unit represented by the following structural formula:

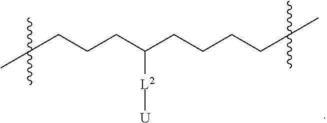

103. The polymer of claim 102, wherein the plurality of first repeat units comprises
a repeat unit represented by the following structural formula:

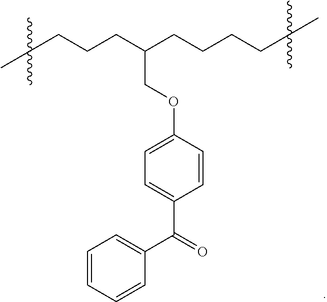

104. The polymer of claim 28, wherein the plurality of second repeat units comprises a repeat unit represented by the following structural formula:

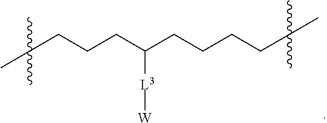

105-106. (canceled)

107. The polymer of claim 28, wherein the plurality of second repeat units comprises a repeat unit represented by any one of the following structural formulas: 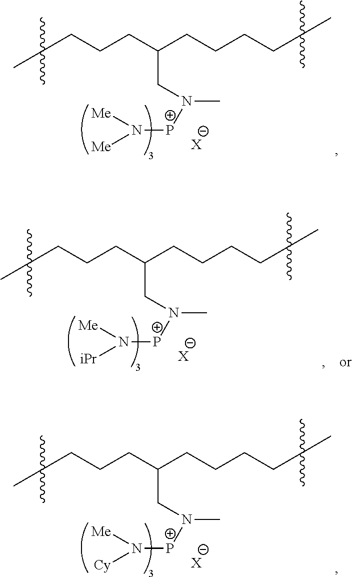
wherein Me is methyl, iPr is isopropyl and Cy is cyclohexyl.

108. The polymer of claim 28, wherein
the plurality of first repeat units comprises a repeat unit represented by the following structural formula: 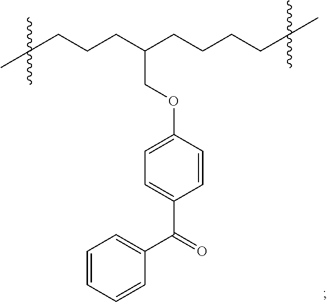
the plurality of second repeat units comprises a repeat unit represented by one of the following structural formulas:

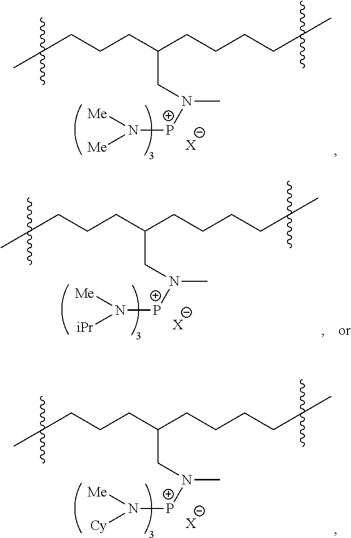

109. The polymer of claim 28, wherein the polymer comprises from about 0.5 mol-% to about 50 mol-% of the first repeat units.

110. (canceled)

111. The polymer of claim 28, wherein the polymer comprises from about 10 mol-% to about 80 mol-% of the second repeat units.

112-114. (canceled)

115. The polymer of claim 28, wherein the polymer is cross-linked.

116. A cross-linked polymer, comprising:
a plurality of first repeat units selected from cross-linking moieties represented by structural formula (IIa) or structural formula (IIb): 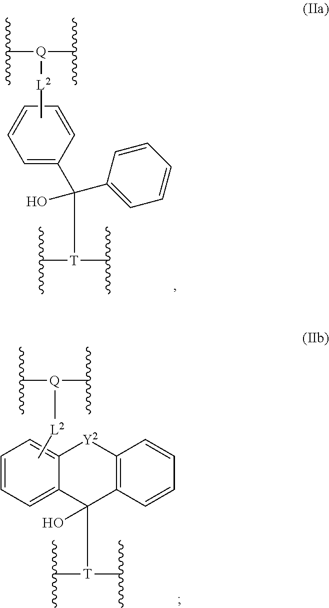
and
a plurality of second repeat units represented by structural formula (III): 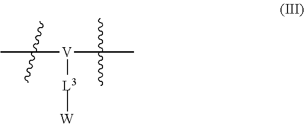
wherein: [symbol]
is a point of attachment to adjacent repeat units of the polymer;
Q is a moiety represented by one of the following structural formulas: 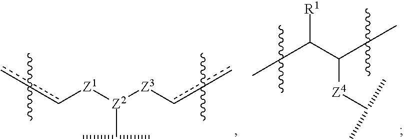
V is a moiety represented by one of the following structural formulas: 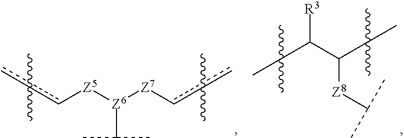
W is a C₁₋₁₂ alkyl or a moiety represented by one of the structural formulas selected from: 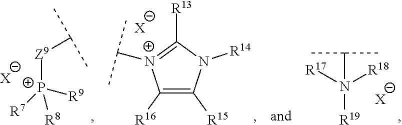
T, for each occurrence independently, is a C₂₋₈ alkylene, [symbol]
is a point of attachment to adjacent repeat units of the polymer; [symbol]
is a point of attachment to L²; [symbol]
is a point of attachment to L³
and further wherein: [symbol]
is a double bond or a single bond;
Z¹, Z³, Z⁵, and Z⁷ each independently is a C₁₋₃ alkylene or a bond;
Z² is selected from —CHR⁵—, a C₅₋₁₂ cycloalkylene, and a 5 to 16-membered heterocyclylene;
Z⁴ is selected from a bond, a C₆₋₁₂ arylene, and CR², wherein
if Z⁴ is CR², then R¹ and R² together with the carbon atoms to which they are attached form a C₅₋₁₂ cycloalkyl or a 5 to 12-membered heterocyclyl, and
if Z⁴ is selected from a bond and a C₆₋₁₂ arylene, then R¹ is selected from H, a C₁₋₁₂ alkyl, and C₆₋₁₂ aryl;
Z⁶ is selected from —CHR⁶—, a C₅₋₁₂ cycloalkylene, and a 5 to 16-membered heterocyclylene;
Z⁸ is selected from a bond, a C₆₋₁₂ arylene, and CR⁴, wherein
if Z⁸ is CR⁴, then R³ and R⁴ together with the carbon atoms to which they are attached form a C₅₋₁₂ cycloalkyl or a 5 to 12 membered heterocyclyl; and
if Z⁸ is selected from a bond and a C₆₋₁₂ arylene, then R³ is selected from H, a C₁₋₁₂ alkyl, and C₆₋₁₂ aryl;
Z⁹ is NR¹⁰ or a bond;
R¹, if present, and R³, if present, each independently is selected from H, a C₁₋₁₂ alkyl, and C₆₋₁₂ aryl;
R⁵ and R⁶ each independently is H or a C₁₋₁₂ alkyl;
R⁷, R⁸, and R⁹ each independently is selected from NR¹¹R¹², a C₆₋₁₂ aryl, and 5 to 12-membered heterocyclyl;
R¹⁰ is a C₁₋₁₂ alkyl;
R¹¹ and R¹² each independently is C₁₋₁₂ alkyl or a C₃₋₁₂ cycloalkyl, or R¹¹ and R¹² together with the nitrogen atom to which they are attached form a 5 to 12-membered heterocyclyl;
R¹³ is selected from a C₁₋₁₂ alkyl, C₆₋₁₂ aryl, and 5 to 12-membered heterocyclyl;
R¹⁴ is a C₁₋₁₂ alkyl or a C₃₋₁₂ cycloalkyl;
R¹⁵ and R¹⁶ each independently is selected from C₁-12 alkyl, C₆₋₁₂ aryl, and 5 to 12-membered heterocyclyl; or R¹⁵ and R¹⁶ together with the carbon atoms to which they are attached form a C₆₋₁₂ aryl or a 5 to 12-membered heterocyclyl; and
R¹⁷, R¹⁸, and R¹⁹ each independently is a C₁-12 alkyl or a C₃₋₁₂ cycloalkyl; or
R¹⁸ and R¹⁹ together with the nitrogen atom to which they are attached form a 5 to 12-membered heterocyclyl and (i) R¹⁷ is a C₁₋₁₂ alkyl or a C₃₋₁₂ cycloalkyl, or (ii) R¹⁷ and at least one atom of L³, if present, together with the nitrogen atom to which L³ and R¹⁷ are attached form a 5- to 12-membered heterocyclyl; or
R¹⁷, R¹⁸, and R¹⁹ together with the nitrogen atom to which they are attached form a bicyclic 5 to 12-membered heterocyclyl; and
L² is selected from a C₁₋₁₂ alkylene, C₆₋₁₂ arylene, C₆₋₁₂ arylene-C₁₋₁₂ alkylene, C₁₋₁₂ alkylene-C₆₋₁₂ arylene, C₁₋₁₂ alkylene-O—C₁₋₁₂ alkylene, (O—C₁₋₁₂ alkylene)ₘ, (C₁₋₁₂ alkylene-O)ₘ, C₁₋₁₂ alkylene-NH—C₁₋₁₂ alkylene, C₁₋₁₂ alkylene-N(C₁₋₁₂ alkyl)-C₁₋₁₂ alkylene, (NH—C₁₋₁₂ alkylene)ₘ, and (C₁₋₁₂ alkylene-NH)ₘ;
L³ is selected from a C₁₋₁₂ alkylene, C₆₋₁₂ arylene, C₆₋₁₂ arylene-C₁₋₁₂ alkylene, C₁₋₁₂ alkylene-C₆₋₁₂ arylene, C₁₋₁₂ alkylene-O—C₁₋₁₂ alkylene, (O—C₁₋₁₂ alkylene)ₙ, (C₁₋₁₂ alkylene-O)ₙ, C₁₋₁₂ alkylene-NH—C₁₋₁₂ alkylene, C₁₋₁₂ alkylene-N(C₁₋₁₂ alkyl)-C₁₋₁₂ alkylene, (NH—C₁₋₁₂ alkylene)ₙ, (C₁₋₁₂ alkylene-NH)ₙ, and a bond;
m is an integer between 1 and 6;
n is an integer between 1 and 6;
Y² is selected from —C(═O)—, O, S, NH, N(C₁₋₁₂ alkyl), and a bond;
X⁻ is selected from F⁻, Cl⁻, Br⁻, OH⁻, NO⁻₂, CN⁻, HCO₃⁻, CO₃²⁻, PF₆⁻, BF₄⁻, a C₁₋₁₂ carboxylate and a C₁₋₁₂ alkoxide;
provided that W is C₁₋₁₂ alkyl only when V is a cationic moiety,
and wherein:
each C₁₋₁₂ alkylene, C₆₋₁₂ arylene, C₅₋₁₂ cycloalkylene, 5 to 16-membered heterocyclylene, C₁₋₁₂ alkyl, C₃₋₁₂ cycloalkyl, C₅₋₁₂ cycloalkyl, C₆₋₁₂ aryl, and 5 to 12-membered heterocyclyl is independently optionally substituted with 1 to 6 substituents independently selected from the group consisting of F, Cl, Br, OH, NH₂, oxo, a C₁₋₁₂ alkyl, C₆₋₁₂ aryl, C₁₋₁₂ haloalkyl, C₁₋₁₂ alkoxy, C₆₋₁₂ aryl, C₆₋₁₂ aryloxy, NH(C₁₋₁₂ alkyl), N(C₁₋₁₂ alkyl)₂, C(O)O(C₁₋₁₂ alkyl), and C(O)NH(C₁₋₁₂ alkyl).

117. The cross-linked polymer of claim 116, wherein
the plurality of first repeat units comprises a cross-linking moiety represented by
the following structural formula: 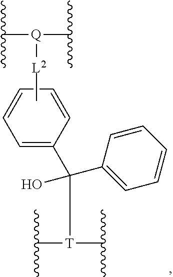
Q is a moiety represented by the following structural formula: 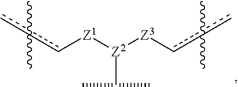
V is a moiety represented by the following structural formula: 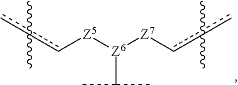
and
W is a C₁₋₁₂ alkyl or a moiety represented by the following structural formula:

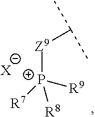

118. The cross-linked polymer of claim 117, wherein
W is a C₁₋₁₂ alkyl or a moiety represented by the following structural formula:

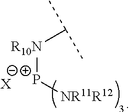

119. The cross-linked polymer of claim 116, wherein
the plurality of first repeat units comprises a cross-linking moiety represented by the following structural formula: 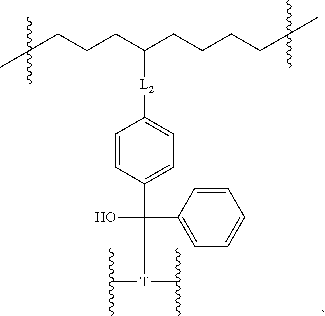
and
the plurality of second repeat units comprises a repeat unit represented by the following structural formula:

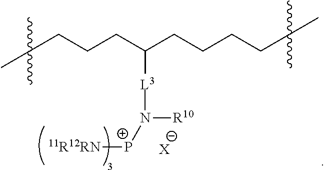

120. The cross-linked polymer of claim 116, wherein
the plurality of first repeat units comprises a cross-linking moiety represented by
the following structural formula: 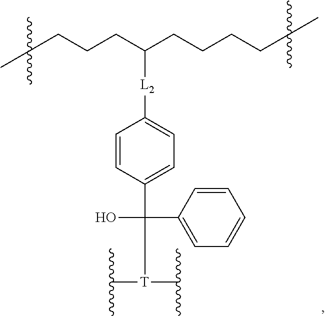
and
the plurality of second repeat units comprises a repeat unit represented by one of the following structural formulas: 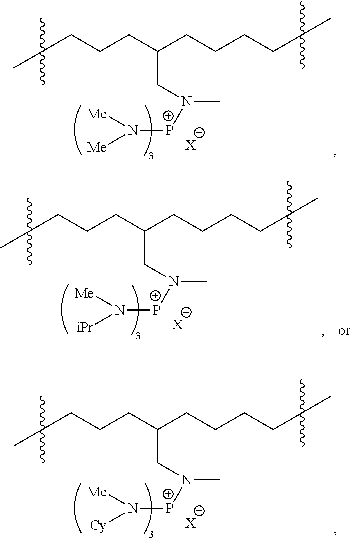
wherein Me is methyl, iPr is isopropyl, and Cy is cyclohexyl.

121. A composite material, comprising a reinforcement material and a a cross-linked polymer claim 116 in contact with said reinforcement material.

122. The composite material of claim 121, wherein:
the reinforcement material is a porous material; and
the porous material is impregnated with the cross-linked polymer.

123. A membrane, comprising a film of the cross-linked polymer of claim 116.

124-126. (canceled)
